(19) United States
(12) Patent Application Publication
(10) Pub. No.: US 2024/0100157 A1
(43) Pub. Date: Mar. 28, 2024

(54) MESOTHELIN BINDING PROTEINS

(71) Applicant: Harpoon Therapeutics, Inc., South San Francisco, CA (US)

(72) Inventors: Holger WESCHE, San Francisco, CA (US); Bryan D. LEMON, Mountain View, CA (US); Richard J. AUSTIN, San Francisco, CA (US); Robert B. DUBRIDGE, Belmont, CA (US)

(21) Appl. No.: 18/184,392

(22) Filed: Mar. 15, 2023

Related U.S. Application Data
(63) Continuation of application No. 16/773,843, filed on Jan. 27, 2020, now Pat. No. 11,607,453.
Application No. 16/773,843 is a continuation of application No. 15/977,968, filed on May 11, 2018, now Pat. No. 10,543,271.
(60) Provisional application No. 62/657,417, filed on Apr. 13, 2018.
(60) Provisional application No. 62/505,719, filed on May 12, 2017.

(51) Int. Cl. A61K 39/395; A61K 39/00; A61P 35/04; C07K 16/18; C07K 16/28; C07K 16/30

(52) U.S. Cl. CPC A61K 39/39558; A61K 39/0011; A61P 35/04; C07K 16/18; C07K 16/28; C07K 16/2809; C07K 16/30; A61K 2039/505; A61K 2039/55511; C07K 2317/22; C07K 2317/24; C07K 2317/31; C07K 2317/569; C07K 2317/73; C07K 2317/732; C07K 2317/734; C07K 2317/92; C07K 2317/94

(57) ABSTRACT

Disclosed herein are MSLN binding proteins with improved binding affinities and improved ability to mediate T cell dependent killing of cancer cells expressing mesothelin. Pharmaceutical compositions comprising the binding proteins disclosed herein and methods of using such formulations are further provided.

### CROSS-REFERENCE

This application is a continuation application of U.S. application Ser. No. 16/773,843, filed Jan. 27, 2020 (now U.S. Pat. No. 11,607,453, issued on Mar. 21, 2023), which is a continuation application of Ser. No. 15/977,968, filed on May 11, 2018 (now U.S. Pat. No. 10,543,271, issued on Jan. 28, 2020), and claims the benefit of U.S. Provisional Application Nos. 62/505,719 filed on May 12, 2017, and 62/657,417 filed Apr. 13, 2018, all of which are incorporated herein by reference in their entirety.

### SEQUENCE LISTING

The instant application contains a Sequence Listing which has been submitted electronically in XML format and is hereby incorporated by reference in its entirety. Said XML copy, created on Apr. 18, 2023, is named 47517-719_302_SL.xml and is 432,263 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if set forth in their entireties.

### BACKGROUND OF THE INVENTION

The present disclosure provides mesothelin (MSLN) binding proteins which can be used for diagnosing and treating indications correlated to the expression of MSLN. Mesothelin (MSLN) is a GPI-linked membrane bound tumor antigen MSLN is overexpressed ovarian, pancreatic, lung and triple-negative breast cancers and mesothelioma. Normal tissue expression of MSLN is restricted to single-cell, mesothelial layers lining the pleural, pericardial, and peritoneal cavities. Overexpression of MSLN is associated with poor prognosis in lung adenocarcinoma and triple-negative breast cancer. MSLN has been used as cancer antigen for numerous modalities, including immunotoxins, vaccines, antibody drug conjugates and CAR-T cells. Early signs of clinical efficacy have validated MSLN as a target, but therapies with improved efficacy are needed to treat MSLN-expressing cancers.

### SUMMARY OF THE INVENTION

One embodiment provides a single domain mesothelin binding protein, wherein said protein comprises one or more conserved regions comprising a sequence identical to or comprising one or more amino acid residue substitutions relative to any one of SEQ ID Nos.: 41-50. In some embodiments, said protein comprises a sequence that is at least 80% identical to a sequence selected from the group consisting of SEQ ID NOs: 1-40, 58, and 60-62. In some embodiments, said protein comprises one or more modifications that result in humanization of the binding protein. In some embodiments, said protein comprises a VHH domain that specifically binds to MSLN. In some embodiments, said protein comprises a VHH domain derived from a non-human source. In some embodiments, said protein comprises a llama VHH domain. In some embodiments, said protein comprises 111 amino acids to 124 amino acids. In some embodiments, said protein binds to an epitope of mesothelin, wherein said epitope is located in region I, comprising amino acid residues 296-390 of SEQ ID NO: 57, region II comprising amino acid residue 391-486 of SEQ ID NO: 57, or region III comprising amino acid residues 487-598 of SEQ ID NO: 57. In some embodiments, said protein comprises one or more CDRs selected from SEQ ID Nos.: 51-56 and 63-179. In some embodiments, said protein comprises an amino acid sequence as set forth in any one of SEQ ID Nos.: 1-40, 58, and 60-62. One embodiment provides a single domain mesothelin binding protein, wherein said protein comprises one or more CDRs selected from SEQ ID Nos.: 51-56 and 63-179. In some embodiments, said protein comprises the following formula:

f1-r1-f2-r2-f3-r3-f4

wherein, r1 CDR1; r2 is CDR2; and r3 CDR3; and wherein f1, f2, f3 and f4 are framework residues.

In some embodiments, said protein comprises a CDR1 comprising a sequence set forth in any one of SEQ ID Nos.: 51, 54, and 63-101. In some embodiments, said protein comprises a CDR2 comprising a sequence set forth in any one of SEQ ID Nos.: 52, 55, and 102-140. In some embodiments, said protein comprises a CDR3 comprising a sequence set forth in any one of SEQ ID Nos.: 53, 56, and 141-179. In some embodiments, f1 comprises a sequence as set forth in any one of SEQ ID Nos.: 180-218. In some embodiments, f2 comprises a sequence as set forth in any one of SEQ ID Nos.: 219-257. In some embodiments, f3 comprises a sequence as set forth in any one of SEQ ID Nos.: 258-296. In some embodiments, f4 comprises a sequence as set forth in any one of SEQ ID Nos.: 297-335. In some embodiments, said protein comprises an amino acid sequence as set forth in any one of SEQ ID Nos.: 1-40, 58, and 60-62.

One embodiment provides a method for the treatment or amelioration of a proliferative disease, or a tumorous disease, comprising the administration of a single domain mesothelin binding protein comprising one or more CDRs selected from SEQ ID Nos.: 51-56 and 63-179, to a subject in need thereof. In some embodiments, the method comprises administering the single domain mesothelin binding protein at a dose of up to 10 mg/kg. In some embodiments, the single domain mesothelin binding protein is administered once a week, twice per week, every other day, or every three weeks. In some embodiments, the subject is human. In some embodiments, the method further comprises administration of an agent in combination with the single domain mesothelin binding protein. In some embodiments, the single domain mesothelin binding protein selectively binds to tumor cells expressing mesothelin. In some embodiments, the single domain mesothelin binding protein mediates T cell killing of tumor cells expressing mesothelin. In some embodiments, the tumorous disease comprises a solid tumor disease. In some embodiments, the solid tumor disease comprises mesothelioma, lung cancer, gastric cancer, ovarian cancer, or triple negative breast cancer. In some embodiments, the solid tumor disease is metastatic.

One embodiment provides a single domain mesothelin binding protein, wherein said protein comprises one or more conserved regions comprising a sequence identical to or comprising one or more amino acid residue substitutions relative to SEQ ID NO: 41, 42, 43, or 44. In some embodiments, said protein comprises a conserved region comprising a sequence identical to or comprising one or more amino acid residue substitutions relative to SEQ ID NO: 41. In some embodiments, said protein comprises a conserved region comprising a sequence identical to or comprising one or more amino acid residue substitutions relative to SEQ ID NO: 42. In some embodiments, said protein comprises a conserved region comprising a sequence identical to or comprising one or more amino acid residue substitutions relative to SEQ ID NO: 43. In some embodiments, said protein comprises a conserved region comprising a sequence identical to or comprising one or more amino acid residue substitutions relative to SEQ ID NO: 44. In some embodiments, said protein comprises (i) a stretch of amino acids corresponding to SEQ ID NO: 41; (ii) a stretch of amino acids corresponding to SEQ ID NO: 42; (iii) a stretch of amino acids corresponding to SEQ ID NO: 43; and (iv) a stretch of amino acids corresponding to SEQ ID NO: 44.

One embodiment provides a single domain mesothelin binding protein, wherein said protein comprises the following formula:

f1-r1-f2-r2-f3-r3-f4

wherein, r1 is identical to or comprises one or more amino acid residue substitutions relative to SEQ ID NO: 51; r2 is identical to or comprises one or more amino acid residue substitutions relative to SEQ ID NO: 52; and r3 is identical to or comprises one or more amino acid residue substitutions relative to SEQ ID NO: 53; and wherein f1, f2, f3 and f4 are framework residues. In some embodiments, said protein comprises a sequence that is at least 80% identical to a sequence selected from the group consisting of SEQ ID NOs: 1-29, 30-40, 58, and 60-62. In some embodiments, said protein comprises one or more modifications that result in humanization of the binding protein. In some embodiments, the modification comprises substitutions, additions, or deletions of amino acid residues. In some embodiments, said protein comprises 111 amino acids to 124 amino acids. In some embodiments, said protein comprises a VHH domain derived from a non-human source. In some embodiments, said protein comprises a llama VHH domain. In some embodiments, said epitope is located in region I, comprising amino acid residues 296-390 of SEQ ID NO: 57, region II comprising amino acid residue 391-486 of SEQ ID NO: 57, or region III comprising amino acid residues 487-598 of SEQ ID NO: 57.

One embodiment provides a single domain mesothelin binding protein, wherein said protein comprises one or more conserved regions comprising a sequence identical to or comprising one or more amino acid residue substitutions relative to SEQ ID NO: 45, 46, 47, 48, 49, or 50. In some embodiments, said protein comprises a conserved region comprising a sequence identical to or comprising one or more amino acid residue substitutions relative to SEQ ID NO: 45. In some embodiments, said protein comprises a conserved region comprising a sequence identical to or comprising one or more amino acid residue substitutions relative to SEQ ID NO: 46. In some embodiments, said protein comprises a conserved region comprising a sequence identical to or comprising one or more amino acid substitutions residue relative to SEQ ID NO: 47. In some embodiments, said protein comprises a conserved region comprising a sequence identical to or comprising one or more amino acid residue substitutions relative to SEQ ID NO: 48. In some embodiments, said protein comprises a conserved region comprising a sequence identical to or comprising one or more amino acid residue substitutions relative to SEQ ID NO: 49. In some embodiments, said protein comprises a conserved region comprising a sequence identical to or comprising one or more amino acid residue substitutions relative to SEQ ID NO: 50. In some embodiments, said protein comprises (i) a stretch of amino acids corresponding to SEQ ID NO: 45; (ii) a stretch of amino acids corresponding to SEQ ID NO: 46; (iii) a stretch of amino acids corresponding to SEQ ID NO: 47, (iv) a stretch of amino acids corresponding to SEQ ID NO: 48, (v) a stretch of amino acids corresponding to SEQ ID NO: 49, and (vi) a stretch of amino acids corresponding to SEQ ID NO: 50.

One embodiment provides a single domain mesothelin binding protein, wherein said protein comprises the following formula:

f1-r1-f2-r2-f3-r3-f4

wherein, r1 is identical to or comprises one or more amino acid residue substitutions relative to SEQ ID NO: 54; r2 is identical to or comprises one or more amino acid residue substitutions relative to SEQ ID NO: 55; and r3 is identical to or comprises one or more amino acid residue substitutions relative to SEQ ID NO: 56; and wherein f1, f2, f3 and f4 are framework residues. In some embodiments, said protein comprises a sequence that is at least 80% identical to a sequence selected from the group consisting of SEQ ID Nos: 30-40, 58, and 60-62. In some embodiments, said protein comprises 111 amino acids to 119 amino acids. In some embodiments, said protein comprises a VHH domain derived from a non-human source. In some embodiments, said protein comprises a llama VHH domain. In some embodiments, said protein binds to a human mesothelin protein comprising the sequence set forth as SEQ ID NO: 57. In some embodiments, said protein binds to an epitope of mesothelin, wherein said epitope is located in region I, comprising amino acid residues 296-390 of SEQ ID NO: 57, region II comprising amino acid residue 391-486 of SEQ ID NO: 57, or region III comprising amino acid residues 487-598 of SEQ ID NO: 57. In some embodiments, said binding protein is a chimeric antibody, or a humanized antibody. In some embodiments, said binding protein is a single domain antibody. In some embodiments, said binding protein is a humanized single domain antibody.

One embodiment provides a single domain mesothelin binding protein, wherein said protein comprises one or more CDRs selected from SEQ ID Nos.: 51-56 and 63-179. In some embodiments, said protein comprises a CDR1 comprising a sequence set forth in any one of SEQ ID Nos.: 51, 54, and 63-101. In some embodiments, said protein comprises a CDR2 comprising a sequence set forth in any one of SEQ ID Nos.: 52, 55, and 102-140. In some embodiments, said protein comprises a CDR3 comprising a sequence set forth in any one of SEQ ID Nos.: 53, 56, and 141-179. In some embodiments, said protein comprises a framework region 1 (f1) comprising a sequence as set forth in any one of SEQ ID Nos.: 180-218. In some embodiments, said protein comprises a framework region 2 (f2) comprising a sequence as set forth in any one of SEQ ID Nos.: 219-257. In some embodiments, said protein comprises a framework region 3 (f3) comprising a sequence as set forth in any one of SEQ ID Nos.: 258-296. In some embodiments, said protein comprises a framework region 4 (f4) comprising a sequence as set forth in any one of SEQ ID Nos.: 297-335. In some embodiments, said protein comprises an amino acid sequence as set forth in any one of SEQ ID Nos.: 1-40, and 58.

One embodiment provides a polynucleotide encoding a single domain mesothelin binding protein according to any one of the above embodiments. A further embodiment provides a vector comprising the polynucleotide of the above embodiment. A further embodiment provides a host cell transformed with the vector according to the above embodiment.

One embodiment provides a pharmaceutical composition comprising (i) a single domain mesothelin binding protein according to any one of the above embodiments, the polynucleotide according to any one of the above embodiments, the vector according to any one of the above embodiments, or the host cell according to any one of the above embodiments, and (ii) a pharmaceutically acceptable carrier.

A further embodiment provides a process for the production of a single domain mesothelin binding protein according to any one of the above embodiments, said process comprising culturing a host transformed or transfected with a vector comprising a nucleic acid sequence encoding a single domain mesothelin binding protein according to any one of the above embodiments under conditions allowing the expression of the mesothelin binding protein and recovering and purifying the produced protein from the culture.

One embodiment provides a method for the treatment or amelioration of a proliferative disease, or a tumorous disease, comprising the administration of the mesothelin binding protein any one of the above embodiments, to a subject in need thereof. In some embodiments, the subject is human. In some embodiments, the method further comprises administration of an agent in combination with the single domain mesothelin binding protein according to any one of the above embodiments. In some embodiments, the single domain mesothelin binding protein selectively binds to tumor cells expressing mesothelin. In some embodiments, the single domain mesothelin binding protein mediates T cell killing of tumor cells expressing mesothelin. In some embodiments, the tumorous disease comprises a solid tumor disease. In some embodiments, the solid tumor disease comprises mesothelioma, lung cancer, gastric cancer, ovarian cancer, or triple negative breast cancer. In some embodiments, the solid tumor disease is metastatic.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the effectivity of exemplary MSLN targeting trispecific molecules (2A2 and 2A4), containing an anti-MSLN binding protein according to the present disclosure, in killing of OVCAR8 cells that expresses the target protein MSLN.

FIG. 2 illustrates that a trispecific MSLN target antigen binding protein containing an exemplary MSLN binding domain of this disclosure (MH6T) directs T cells from five donors (donor 02; donor 86; donor 41; donor 81; and donor 35) to kill Caov3 cells. The figure also illustrates that a control trispecific protein (GFP TriTAC) did not direct T cells from the five donors (donor 02; donor 86; donor 41; donor 81; and donor 35) to kill Caov3 cells.

FIG. 3 illustrates that a trispecific MSLN target antigen binding protein containing an exemplary MSLN binding domain of this disclosure (MH6T) directs T cells from five donors (donor 02; donor 86; donor 41; donor 81; and donor 35) to kill OVCAR3 cells. The figure also illustrates that a control trispecific protein (GFP TriTAC) did not direct T cells from the five donors (donor 02; donor 86; donor 41; donor 81; and donor 35) to kill OVCAR3 cells.

FIG. 4 illustrates that a trispecific MSLN target antigen binding protein containing an exemplary MSLN binding domain of this disclosure (MH6T) was able to direct T cells from a healthy donor to kill cells that express MSLN (OVCAR3 cells; Caov4 cells; OVCAR3 cells; and OVCAR8 cells). The figure also illustrates that the trispecific MSLN target antigen binding protein containing an exemplary MSLN binding domain of this disclosure (MH6T) was not able to direct T cells from the healthy donor to kill cells that do not express MSLN (MDAPCa2b cells; and NCI-H510A cells).

FIG. 5 illustrates that a trispecific MSLN target antigen binding protein containing an exemplary MSLN binding domain of this disclosure (MH6T) was able to direct T cells from cynomolgus monkeys to kill human ovarian cancer cells (OVCAR3 cells; Caov3 cells). The figure also illustrates that a control trispecific protein (GFP TriTAC) was not able to direct the T cells from cynomolgus monkeys to kill human ovarian cancer cells lines (OVCAR3 cells; Caov3 cells).

FIG. 6 illustrates that a trispecific MSLN target antigen binding protein containing an exemplary MSLN binding domain of this disclosure (MH6T) was able to direct killing of MSLN expressing NCI-H2052 mesothelioma cells by T cells, in the presence or absence of human serum albumin (HSA).

FIG. 7 illustrates that a trispecific MSLN target antigen binding protein containing an exemplary MSLN binding domain of this disclosure (MH6T) was able to activate T cells from four healthy donors (donor 2; donor 86; donor 35; and donor 81), as demonstrated by secretion of TNF-α from the T cells, in presence of MSLN-expressing Caov4 cells.

FIG. 8 illustrates that a trispecific MSLN target antigen binding protein containing an exemplary MSLN binding domain of this disclosure (MH6T) was able to activate T cells from four healthy donors (donor 2; donor 86; donor 35; and donor 81), as demonstrated by activation of CD69 expression on the T cells, in presence of MSLN-expressing OVCAR8 cells.

FIG. 9A and FIG. 9B illustrates binding of a trispecific MSLN target antigen binding protein containing an exemplary MSLN binding domain of this disclosure (MH6T) to MSLN expressing cell lines or MSLN non-expressing cell lines. FIG. 9A shows binding with MSLN expressing cells (Caov3 cells-top left panel; Caov4 cells-top right panel; OVCAR3 cells-bottom left panel; OVCAR8 cells-bottom right panel) bound to the trispecific MSLN target antigen binding protein containing an exemplary MSLN binding domain of this disclosure (MH6T); and further illustrates lack of binding of a control trispecific protein (GFP TriTAC) to the same cell lines. FIG. 9B shows lack of binding of both the trispecific MSLN target antigen binding protein containing an exemplary MSLN binding domain of this disclosure (MH6T) and the GFP TriTAC to MSLN non-expressing cell lines (MDCA2b cells-left panel; NCI-H510A cells-right panel).

FIG. 10 illustrates binding of a trispecific MSLN target antigen binding protein containing an exemplary MSLN binding domain of this disclosure (MH6T) to T cells from four healthy donors (donor 2-top left panel; donor 35-top right panel; donor 41-bottom left panel; donor 81-bottom right panel).

FIG. 11 illustrates that a trispecific MSLN target antigen binding protein containing an exemplary MSLN binding domain of this disclosure (MH6T) was able to inhibit tumor growth in NCG mice implanted with MSLN expressing NCI-H292 cells.

FIG. 12 illustrates pharmacokinetic profile of a trispecific MSLN target antigen binding protein containing an exemplary MSLN binding domain of this disclosure (MH6T). Serum levels of the trispecific MSLN target antigen binding protein containing an exemplary MSLN binding domain of this disclosure (MH6T), at various time points following injection into two cynomolgus monkeys, are shown in the plot.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

The term “Framework” or “FR” residues (or regions) refer to variable domain residues other than the CDR or hypervariable region residues as herein defined. A “human consensus framework” is a framework which represents the most commonly occurring amino acid residue in a selection of human immunoglobulin VL or VH framework sequences.

As used herein, “Variable region” or “variable domain” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the βsheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity. “Variable domain residue numbering as in Kabat” or “amino acid position numbering as in Kabat,” and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence. It is not intended that CDRs of the present disclosure necessarily correspond to the Kabat numbering convention.

As used herein, the term “Percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer softwares such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

As used herein, “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2} the time required for 50% completion of the process. The units of these two constants are time⁻¹ and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e. after drug absorption and distribution are complete). The half-time for drug elimination can be accurately determined from such a graph.

As used herein, the term “binding affinity” refers to the affinity of the proteins described in the disclosure to their binding targets, and is expressed numerically using “Kd” values. If two or more proteins are indicated to have comparable binding affinities towards their binding targets, then the Kd values for binding of the respective proteins towards their binding targets, are within ±2-fold of each other. If two or more proteins are indicated to have comparable binding affinities towards single binding target, then the Kd values for binding of the respective proteins towards said single binding target, are within ±2-fold of each other. If a protein is indicated to bind two or more targets with comparable binding affinities, then the Kd values for binding of said protein to the two or more targets are within ±2-fold of each other. In general, a higher Kd value corresponds to a weaker binding. In some embodiments, the “Kd” is measured by a radiolabeled antigen binding assay (MA) or surface plasmon resonance assays using a BIAcore™-2000 or a BIAcore™-3000 (BIAcore, Inc., Piscataway, N.J.). In certain embodiments, an “on-rate” or “rate of association” or “association rate” or “kon” and an “off-rate” or “rate of dissociation” or “dissociation rate” or “koff” are also determined with the surface plasmon resonance technique using a BIAcore™-2000 or a BIAcore™-3000 (BIAcore, Inc., Piscataway, N.J.). In additional embodiments, the “Kd”, “kon”, and “koff” are measured using the OCTET® Systems (Pall Life Sciences). In an exemplary method for measuring binding affinity using the OCTET® Systems, the ligand, e.g., biotinylated human or cynomolgus MSLN, is immobilized on the OCTET® streptavidin capillary sensor tip surface which streptavidin tips are then activated according to manufacturer's instructions using about 20-50 μg/ml human or cynomolgus MSLN protein. A solution of PBS/Casein is also introduced as a blocking agent. For association kinetic measurements, MSLN binding protein variants are introduced at a concentration ranging from about 10 ng/mL to about 100 μg/mL, about 50 ng/mL to about 5 μg/mL, or about 2 ng/mL to about 20 μg/mL. In some embodiments, the MSLN binding single domain proteins are used at a concentration ranging from about 2 ng/mL to about 20 μg/mL. Complete dissociation is observed in case of the negative control, assay buffer without the binding proteins. The kinetic parameters of the binding reactions are then determined using an appropriate tool, e.g., ForteBio software.

Described herein are MSLN binding proteins, pharmaceutical compositions as well as nucleic acids, recombinant expression vectors, and host cells for making such MSLN binding proteins. Also provided are methods of using the disclosed MSLN binding proteins in the prevention, and/or treatment of diseases, conditions and disorders. The MSLN binding proteins are capable specifically binding to MSLN. In some embodiments, the MSLN binding proteins include additional domains, such as a CD3 binding domain and an albumin binding domain.

### Mesothelin (MSLN) and its Role in Tumorous Diseases

Contemplated herein are mesothelin binding proteins. Mesothelin is a glycoprotein present on the surface of cells of the mesothelial lining of the peritoneal, pleural and pericardial body cavities. The mesothelin gene (MSLN) encodes a 71-kilodalton (kDa) precursor protein that is processed to a 40-kDa protein termed mesothelin, which is a glycosyl-phosphatidylinositol-anchored glycoprotein present on the cell surface (Chang, et al, Proc Natl Acad Sci USA (1996) 93:136-40). The mesothelin cDNA was cloned from a library prepared from the HPC-Y5 cell line (Kojima et al. (1995) J. Biol. Chem. 270:21984-21990). The cDNA also was cloned using the monoclonal antibody K1, which recognizes mesotheliomas (Chang and Pastan (1996) Proc. Natl. Acad. Sci. USA 93:136-40). Mesothelin is a differentiation antigen whose expression in normal human tissues is limited to mesothelial cells lining the body cavity, such as the pleura, pericardium and peritoneum. Mesothelin is also highly expressed in several different human cancers, including mesotheliomas, pancreatic adenocarcinomas, ovarian cancers, stomach and lung adenocarcinomas. (Hassan, et al., Eur J Cancer (2008) 44:46-53) (Ordonez, Am J Surg Pathol (2003) 27:1418-28; Ho, et al., Clin Cancer Res (2007) 13:1571-5). Mesothelin is overexpressed in a vast majority of primary pancreatic adenocarcinomas with rare and weak expression seen in benign pancreatic tissue. Argani P, et al. Clin Cancer Res. 2001; 7(12):3862-3868. Epithelial malignant pleural mesothelioma (MPM) universally expresses mesothelin while sarcomatoid MPM likely does not express mesothelin. Most serous epithelial ovarian carcinomas, and the related primary peritoneal carcinomas, express mesothelin.

Mesothelin is also shed from tumor cells as a soluble form of the protein, as compared to the native membrane bound version (Hellstrom, et al., Cancer Epidemiol Biomarkers Prev (2006) 15:1014-20; Ho, et al., Cancer Epidemiol Biomarkers Prev (2006) 15:1751). Structurally, mesothelin is expressed on the cell surface as a 60 kDa precursor polypeptide, which is proteolytically processed into a 31 kDa shed component (corresponding to MPF) and a 40 kDa membrane bound component (Hassan et al. (2004) Clin. Cancer. Res. 10:3937-3942). Mesothelin has been shown to interact with CA125 (also known as MUC-16), a mucin-like glycoprotein present on the surface of tumor cells that previously had been identified as an ovarian cancer antigen. Further, binding of CA125 to membrane-bound mesothelin mediates heterotypic cell adhesion and CA125 and mesothelin are co-expressed in advanced grade ovarian adenocarcinoma (Rump, A. et al. (2004) J. Biol. Chem. 279:9190-9198). Expression of mesothelin in the lining of the peritoneum correlates with the preferred site of metastasis formation of ovarian cancer and mesothelin-CA125 binding is thought to facilitate peritoneal metastasis of ovarian tumors (Gubbels, J. A. et al. (2006) Mol. Cancer. 5:50).

Mesothelin is a target of a natural immune response in ovarian cancer, and has been proposed to be a target for cancer immunotherapy. Bracci L, et al. Clin Cancer Res. 2007; 13(2 Pt 1):644-653; Moschella F, et al. Cancer Res. 2011; 71(10):3528-3539; Gross G, et al. FASEB J. 1992; 6(15):3370-3378; Sadelain M, et al. Nat Rev Cancer. 2003; 3(1):35-45; Muul L M, et al. Blood. 2003; 101(7):2563-2569; Yee C, et al. Proc Natl Acad Sci USA. 2002; 99(25):16168-16173. The presence of mesothelin-specific CTLs in patients with pancreatic cancer correlates with overall survival. Thomas A M, et al. J Exp Med. 2004; 200:297-306. In addition, Pastan and coworkers have used soluble antibody fragments of an anti-mesothelin antibody conjugated to immunotoxins to treat cancer patients with mesothelin-positive tumors. This approach has demonstrated adequate safety and some clinical activity in pancreatic cancer. Hassan R, et al. Cancer Immun. 2007; 7:20 and Hassan R, et al. Clin Cancer Res. 2007; 13(17):5144-5149. In ovarian cancer, this therapeutic strategy produced one minor response by RECIST criteria and stable disease in a second patient who also had complete resolution of their ascites.

Mesothelin can also be used a marker for diagnosis and prognosis of certain types of cancer because trace amounts of mesothelin can be detected in the blood of some patients with mesothelin-positive cancers (Cristaudo et al., Clin. Cancer Res. 13:5076-5081, 2007). It has been reported that mesothelin may be released into serum through deletion at its carboxyl terminus or by proteolytic cleavage from its membrane bound form (Hassan et al., Clin. Cancer Res. 10:3937-3942, 2004). An increase in the soluble form of mesothelin was detectable several years before malignant mesotheliomas occurred among workers exposed to asbestos (Creaney and Robinson, Hematol. Oncol. Clin. North Am. 19:1025-1040, 2005). Furthermore, patients with ovarian, pancreatic, and lung cancers also have elevated soluble mesothelin in serum (Cristaudo et al., Clin. Cancer Res. 13:5076-5081, 2007; Hassan et al., Clin. Cancer Res. 12:447-453, 2006; Croso et al., Cancer Detect. Prev. 30:180-187, 2006). Accordingly, mesothelin is an appropriate target for methods of disease prevention or treatment and there is a need for effective antibodies specific for mesothelin.

It has been shown that cell surface mature mesothelin comprises three distinct domains, namely Regions I (comprising residues 296-390), II (comprising residues 391-486), and III (comprising residue 487-598). (Tang et al., A human single-domain antibody elicits potent antitumor activity by targeting an epitope in mesothelin close to the cancer cell surface, Mol. Can. Therapeutics, 12(4): 416-426, 2013).

The first antibodies generated against mesothelin for therapeutic intervention were designed to interfere with the interaction between mesothelin and CA-125. Phage display identified the Fv SS, which was affinity optimized and used to generate a recombinant immunotoxin targeting mesothelin, SS1P. The MORAb-009 antibody amatuximab, which also uses SS1, recognizes a non-linear epitope in the amino terminal 64 amino acids of mesothelin, within region I. The SS1 Fv was also used to generate chimeric antigen receptor-engineered T cells. Recently, new anti-mesothelin antibodies have been reported that recognize other regions of the mesothelin protein.

There is still a need for having available further options for the treatment of solid tumor diseases related to the overexpression of mesothelin, such as ovarian cancer, pancreatic cancer, mesothelioma, lung cancer, gastric cancer and triple negative breast cancer. The present disclosure provides, in certain embodiments, single domain proteins which specifically bind to MSLN on the surface of tumor target cells.

### MSLN Binding Proteins

Provided herein in certain embodiments are binding proteins, such as anti-MSLN single domain antibodies or antibody variants, which bind to an epitope in the MSLN protein. In some embodiments, the MSLN binding protein binds to a protein comprising the sequence of SEQ ID NO: 57. In some embodiments, the MSLN binding protein binds to a protein comprising a truncated sequence compared to SEQ ID NO: 57.

In some embodiments, the MSLN binding proteins disclosed herein recognize full-length mesothelin. In certain instances, the MSLN binding proteins disclosed herein recognize an epitope in region I (comprising amino acid residues 296-390 of SEQ ID NO: 57), region II (comprising amino acid residue 391-486 of SEQ ID NO: 57), or region III (comprising amino acid residues 487-598 of SEQ ID NO: 57) of mesothelin. It is contemplated that the MSLN binding proteins of the present disclosure may, in some embodiments, recognize and bind to epitopes that are located outside regions I, II, or III of mesothelin. In yet other embodiments are disclosed MSLN binding proteins that recognize and bind to an epitope different than the MORAb-009 antibody.

In some embodiments, the MSLN binding proteins of the present disclosure are expressed within a multidomain protein that includes additional immunoglobulin domains. Such multidomain proteins can act via immunotoxin-based inhibition of tumor growth and induction of antibody-dependent cellular cytotoxicity (ADCC). In some embodiments, the multidomain proteins containing the MSLN binding proteins of the present disclosure exhibit complement-dependent cytotoxicity (CDC) activity. In some embodiments, the multidomain proteins containing the MSLN binding proteins of the present disclosure exhibit both ADCC and CDC activity, against cancer cells expressing mesothelin.

Furthermore, in some embodiments, where multidomain proteins containing the MSLN binding proteins act via CDC, the MLSN binding protein may recognize a conformational epitope at the C-terminal end of mesothelin protein, close to the cell surface. In some embodiments, the mesothelin protein comprises the sequence as set forth in SEQ ID NO: 57, and the C-terminal end comprises the amino acid residues 539-588.

In some embodiments, the MSLN binding protein is an anti-MSLN antibody or an antibody variant. As used herein, the term “antibody variant” refers to variants and derivatives of an antibody described herein. In certain embodiments, amino acid sequence variants of the anti-MSLN antibodies described herein are contemplated. For example, in certain embodiments amino acid sequence variants of anti-MSLN antibodies described herein are contemplated to improve the binding affinity and/or other biological properties of the antibodies. Exemplary method for preparing amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to arrive at the final construct, provided that the final construct possesses the desired characteristics, e.g., antigen-binding. In certain embodiments, antibody variants having one or more amino acid substitutions are provided. Sites of interest for substitution mutagenesis include the CDRs and framework regions. Examples of such substitutions are described below. Amino acid substitutions may be introduced into an antibody of interest and the products screened for a desired activity, e.g., retained/improved antigen binding, decreased immunogenicity, or improved antibody-dependent cell mediated cytotoxicity (ADCC) or complement dependent cytotoxicity (CDC). Both conservative and non-conservative amino acid substitutions are contemplated for preparing the antibody variants.

In another example of a substitution to create a variant anti-MSLN antibody, one or more hypervariable region residues of a parent antibody are substituted. In general, variants are then selected based on improvements in desired properties compared to a parent antibody, for example, increased affinity, reduced affinity, reduced immunogenicity, increased pH dependence of binding. For example, an affinity matured variant antibody can be generated, e.g., using phage display-based affinity maturation techniques such as those described herein and known in the field.

In some embodiments, the MSLN binding protein described herein is a single domain antibody such as a heavy chain variable domain (VH), a variable domain (VHH) of llama derived sdAb, peptide, ligand or a small molecule entity specific for mesothelin. In some embodiments, the mesothelin binding domain of the MSLN binding protein described herein is any domain that binds to mesothelin including but not limited to domains from a monoclonal antibody, a polyclonal antibody, a recombinant antibody, a human antibody, a humanized antibody. In certain embodiments, the MSLN binding protein is a single-domain antibody. In other embodiments, the MSLN binding protein is a peptide. In further embodiments, the MSLN binding protein is a small molecule.

Generally, it should be noted that the term single domain antibody as used herein in its broadest sense is not limited to a specific biological source or to a specific method of preparation. Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. For example, in some embodiments, the single domain antibodies of the disclosure are obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring VH domain from any animal species, and in particular from a species of mammal, such as from a human being, or by expression of a nucleic acid encoding such a camelized VH domain; (5) by “camelisation” of a “domain antibody” or “Dab”, or by expression of a nucleic acid encoding such a camelized VH domain; (6) by using synthetic or semi-synthetic techniques for preparing proteins, polypeptides or other amino acid sequences; (7) by preparing a nucleic acid encoding a single domain antibody using techniques for nucleic acid synthesis known in the field, followed by expression of the nucleic acid thus obtained; and/or (8) by any combination of one or more of the foregoing.

In one embodiment, a single domain antibody corresponds to the VHH domains of naturally occurring heavy chain antibodies directed against MSLN. As further described herein, such VHH sequences can generally be generated or obtained by suitably immunizing a species of Llama with MSLN, (i.e., so as to raise an immune response and/or heavy chain antibodies directed against MSLN), by obtaining a suitable biological sample from said Llama (such as a blood sample, serum sample or sample of B-cells), and by generating VHH sequences directed against MSLN, starting from said sample, using any suitable technique known in the field.

In another embodiment, such naturally occurring VHH domains against MSLN, are obtained from naïve libraries of Camelid VHH sequences, for example by screening such a library using MSLN, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from naïve VHH libraries are used, such as VHH libraries obtained from naïve VHH libraries by techniques such as random mutagenesis and/or CDR shuffling, as for example described in WO 00/43507.

In a further embodiment, yet another technique for obtaining VHH sequences directed against MSLN, involves suitably immunizing a transgenic mammal that is capable of expressing heavy chain antibodies (i.e., so as to raise an immune response and/or heavy chain antibodies directed against MSLN), obtaining a suitable biological sample from said transgenic mammal (such as a blood sample, serum sample or sample of B-cells), and then generating VHH sequences directed against MSLN, starting from said sample, using any suitable technique known in the field. For example, for this purpose, the heavy chain antibody-expressing rats or mice and the further methods and techniques described in WO 02/085945 and in WO 04/049794 can be used.

In some embodiments, an anti-MSLN antibody, as described herein comprises single domain antibody with an amino acid sequence that corresponds to the amino acid sequence of a naturally occurring VHH domain, but that has been “humanized”, i.e., by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g., as indicated above). This can be performed in a manner known in the field, which will be clear to the skilled person, for example on the basis of the further description herein. Again, it should be noted that such humanized anti-MSLN single domain antibodies of the disclosure are obtained in any suitable manner known per se (i.e., as indicated under points (1)-(8) above) and thus are not strictly limited to polypeptides that have been obtained using a polypeptide that comprises a naturally occurring VHH domain as a starting material. In some additional embodiments, a single domain MSLN antibody, as described herein, comprises a single domain antibody with an amino acid sequence that corresponds to the amino acid sequence of a naturally occurring VH domain, but that has been “camelized”, i.e., by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. Such “camelizing” substitutions are preferably inserted at amino acid positions that form and/or are present at the VH-VL interface, and/or at the so-called Camelidae hallmark residues (see for example WO 94/04678 and Davies and Riechmann (1994 and 1996)). Preferably, the VH sequence that is used as a starting material or starting point for generating or designing the camelized single domain is preferably a VH sequence from a mammal, more preferably the VH sequence of a human being, such as a VH3 sequence. However, it should be noted that such camelized anti-MSLN single domain antibodies of the disclosure, in certain embodiments, is obtained in any suitable manner known in the field (i.e., as indicated under points (1)-(8) above) and thus are not strictly limited to polypeptides that have been obtained using a polypeptide that comprises a naturally occurring VH domain as a starting material. For example, as further described herein, both “humanization” and “camelization” is performed by providing a nucleotide sequence that encodes a naturally occurring VHH domain or VH domain, respectively, and then changing, one or more codons in said nucleotide sequence in such a way that the new nucleotide sequence encodes a “humanized” or “camelized” single domain antibody, respectively. This nucleic acid can then be expressed, so as to provide the desired anti-MSLN single domain antibody of the disclosure. Alternatively, in other embodiments, based on the amino acid sequence of a naturally occurring VHH domain or VH domain, respectively, the amino acid sequence of the desired humanized or camelized anti-MSLN single domain antibody of the disclosure, respectively, are designed and then synthesized de novo using known techniques for peptide synthesis. In some embodiments, based on the amino acid sequence or nucleotide sequence of a naturally occurring VHH domain or VH domain, respectively, a nucleotide sequence encoding the desired humanized or camelized anti-MSLN single domain antibody of the disclosure, respectively, is designed and then synthesized de novo using known techniques for nucleic acid synthesis, after which the nucleic acid thus obtained is expressed in using known expression techniques, so as to provide the desired anti-MSLN single domain antibody of the disclosure.

Other suitable methods and techniques for obtaining the anti-MSLN single domain antibody of the disclosure and/or nucleic acids encoding the same, starting from naturally occurring VH sequences or VHH sequences for example comprises combining one or more parts of one or more naturally occurring VH sequences (such as one or more framework (FR) sequences and/or complementarity determining region (CDR) sequences), one or more parts of one or more naturally occurring VHH sequences (such as one or more FR sequences or CDR sequences), and/or one or more synthetic or semi-synthetic sequences, in a suitable manner, so as to provide an anti-MSLN single domain antibody of the disclosure or a nucleotide sequence or nucleic acid encoding the same.

It is contemplated that in some embodiments the MSLN binding protein is fairly small and no more than 25 kD, no more than 20 kD, no more than 15 kD, or no more than 10 kD in some embodiments. In certain instances, the MSLN binding protein is 5 kD or less if it is a peptide or small molecule entity.

In some embodiments, the MSLN binding protein is an anti-MSLN specific antibody comprising a heavy chain variable complementarity determining regionCDR1, a heavy chain variable CDR2, a heavy chain variable CDR3, a light chain variable CDR1, a light chain variable CDR2, and a light chain variable CDR3. In some embodiments, the MSLN binding protein comprises any domain that binds to MSLN including but not limited to domains from a monoclonal antibody, a polyclonal antibody, a recombinant antibody, a human antibody, a humanized antibody, or antigen binding fragments such as single domain antibodies (sdAb), Fab, Fab′, F(ab)2, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain. In some embodiments, the MSLN binding protein is a single domain antibody. In some embodiments, the anti-MSLN single domain antibody comprises heavy chain variable complementarity determining regions (CDR), CDR1, CDR2, and CDR3.

In some embodiments, the MSLN binding protein of the present disclosure is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the MSLN binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues. In some embodiments, the MSLN binding protein comprises an amino acid sequence selected from SEQ ID NOs: 1-40.

In some embodiments, the CDR1 comprises the amino acid sequence as set forth in SEQ ID NO: 51 or a variant having one, two, three, four, five, six, seven, eight, nine, or ten amino acid substitutions in SEQ ID NO: 51. In some embodiments, the CDR2 comprises a sequence as set forth in SEQ ID NO: 52 or a variant having one, two, three, four, five, six, seven, eight, nine, or ten amino acid substitutions in SEQ ID NO: 52. In some embodiments, the CDR3 comprises a sequence as set forth in SEQ ID NO: 53 or a variant having one, two, three, four, five, six, seven, eight, nine, or ten amino acid substitutions in SEQ ID NO: 53.

In some embodiments, the CDR1 comprises the amino acid sequence as set forth in SEQ ID NO: 54 or a variant having one, two, three, four, five, six, seven, eight, nine, or ten amino acid substitutions in SEQ ID NO: 54. In some embodiments, the CDR2 comprises a sequence as set forth in SEQ ID NO: 55 or a variant having one, two, three, four, five, six, seven, eight, nine, or ten amino acid substitutions in SEQ ID NO: 55. In some embodiments, the CDR3 comprises a sequence as set forth in SEQ ID NO: 56 or a variant having one, two, three, four, five, six, seven, eight, nine, or ten amino acid substitutions in SEQ ID NO: 56.

The MSLN binding proteins of the present disclosure, in certain examples, comprise one or more conserved regions. The conserved regions comprise sequences as set forth in SEQ ID NOs: 41-50, or variants comprising one or more amino acid residue substitutions relative to said sequences. Exemplary embodiments include MSLN binding proteins comprising one or more conserved regions selected from SEQ ID NOs: 41-44, or variants comprising one or more amino acid residue substitutions relative to said sequences. In some cases, the MSLN binding protein comprises (i) a stretch of amino acids corresponding to SEQ ID NO: 41, (ii) a stretch of amino acids corresponding to SEQ ID NO: 42, iii) a stretch of amino acids corresponding to SEQ ID NO: 43, and (iv) a stretch of amino acids corresponding to SEQ ID NO: 44.

Further exemplary embodiments include MSLN binding proteins comprising one or more conserved regions selected from SEQ ID NOs: 45-50, or variants comprising one or more amino acid residue substitutions relative to said sequences. In some cases, the MSLN binding protein comprises (i) a stretch of amino acids corresponding to SEQ ID NO: 45, (ii) a stretch of amino acids corresponding to SEQ ID NO: 46, (iii) a stretch of amino acids corresponding to SEQ ID NO: 47, (iv) a stretch of amino acids corresponding to SEQ ID NO: 48, (v) a stretch of amino acid corresponding to SEQ ID NO: 49, and (vi) a stretch of amino acids corresponding to SEQ ID NO: 50.

In various embodiments, the MSLN binding protein of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOs: 1-29, 58, and 60-62.

In various embodiments, the MSLN binding protein of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from SEQ ID NOs: 30-40, 58, and 60-62.

In various embodiments, a complementarity determining region of the MSLN binding protein of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 51, or SEQ ID NO: 54.

In various embodiments, a complementarity determining region of the MSLN binding protein of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 52, or SEQ ID NO: 55.

In various embodiments, a complementarity determining region of the MSLN binding protein of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 53, or SEQ ID NO: 56.

In various embodiments, a complementarity determining region 1 (CDR1) of the MSLN binding protein of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence as set forth in any one of SEQ ID Nos.: 63-101.

In various embodiments, a complementarity determining region 2 (CDR2) of the MSLN binding protein of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence as set forth in any one of SEQ ID Nos.: 102-140.

In various embodiments, a complementarity determining region 3 (CDR3) of the MSLN binding protein of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence as set forth in any one of SEQ ID Nos.: 141-179.

In various embodiments, a framework region 1 (f1) of the MSLN binding protein of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence as set forth in any one of SEQ ID Nos.: 180-218.

In various embodiments, a framework region 1 (f1) of the MSLN binding protein of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence as set forth in any one of SEQ ID Nos.: 219-257.

In various embodiments, a framework region 2 (f2) of the MSLN binding protein of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence as set forth in any one of SEQ ID Nos.: 258-296.

In various embodiments, a framework region 3 (f3) of the MSLN binding protein of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence as set forth in any one of SEQ ID Nos.: 297-335.

In some embodiments, the MSLN binding protein, according to any one of the above embodiments, is a single domain antibody comprising the sequence of SEQ ID NO: 1. In some embodiments, the MSLN binding protein, according to any one of the above embodiments, is a single domain antibody comprising the sequence of SEQ ID NO: 2. In some embodiments, the MSLN binding protein, according to any one of the above embodiments, is a single domain antibody comprising the sequence of SEQ ID NO: 3. In some embodiments, the MSLN binding protein, according to any one of the above embodiments, is a single domain antibody comprising the sequence of SEQ ID NO: 4. In some embodiments, the MSLN binding protein, according to any one of the above embodiments, is a single domain antibody comprising the sequence of SEQ ID NO: 5. In some embodiments, the MSLN binding protein, according to any one of the above embodiments, is a single domain antibody comprising the sequence of SEQ ID NO: 6. In some embodiments, the MSLN binding protein, according to any one of the above embodiments, is a single domain antibody comprising the sequence of SEQ ID NO: 7. In some embodiments, the MSLN binding protein, according to any one of the above embodiments, is a single domain antibody comprising the sequence of SEQ ID NO: 8. In some embodiments, the MSLN binding protein, according to any one of the above embodiments, is a single domain antibody comprising the sequence of SEQ ID NO: 9. In some embodiments, the MSLN binding protein, according to any one of the above embodiments, is a single domain antibody comprising the sequence of SEQ ID NO: 10. In some embodiments, the MSLN binding protein, according to any one of the above embodiments, is a single domain antibody comprising the sequence of SEQ ID NO: 11. In some embodiments, the MSLN binding protein, according to any one of the above embodiments, is a single domain antibody comprising the sequence of SEQ ID NO: 12. In some embodiments, the MSLN binding protein, according to any one of the above embodiments, is a single domain antibody comprising the sequence of SEQ ID NO: 13. In some embodiments, the MSLN binding protein, according to any one of the above embodiments, is a single domain antibody comprising the sequence of SEQ ID NO: 14. In some embodiments, the MSLN binding protein, according to any one of the above embodiments, is a single domain antibody comprising the sequence of SEQ ID NO: 15. In some embodiments, the MSLN binding protein, according to any one of the above embodiments, is a single domain antibody comprising the sequence of SEQ ID NO: 16. In some embodiments, the MSLN binding protein, according to any one of the above embodiments, is a single domain antibody comprising the sequence of SEQ ID NO: 17. In some embodiments, the MSLN binding protein, according to any one of the above embodiments, is a single domain antibody comprising the sequence of SEQ ID NO: 18. In some embodiments, the MSLN binding protein, according to any one of the above embodiments, is a single domain antibody comprising the sequence of SEQ ID NO: 19. In some embodiments, the MSLN binding protein, according to any one of the above embodiments, is a single domain antibody comprising the sequence of SEQ ID NO: 20. In some embodiments, the MSLN binding protein, according to any one of the above embodiments, is a single domain antibody comprising the sequence of SEQ ID NO: 21. In some embodiments, the MSLN binding protein, according to any one of the above embodiments, is a single domain antibody comprising the sequence of SEQ ID NO: 22. In some embodiments, the MSLN binding protein, according to any one of the above embodiments, is a single domain antibody comprising the sequence of SEQ ID NO: 23. In some embodiments, the MSLN binding protein, according to any one of the above embodiments, is a single domain antibody comprising the sequence of SEQ ID NO: 24. In some embodiments, the MSLN binding protein, according to any one of the above embodiments, is a single domain antibody comprising the sequence of SEQ ID NO: 25. In some embodiments, the MSLN binding protein, according to any one of the above embodiments, is a single domain antibody comprising the sequence of SEQ ID NO: 26. In some embodiments, the MSLN binding protein, according to any one of the above embodiments, is a single domain antibody comprising the sequence of SEQ ID NO: 27. In some embodiments, the MSLN binding protein, according to any one of the above embodiments, is a single domain antibody comprising the sequence of SEQ ID NO: 28. In some embodiments, the MSLN binding protein, according to any one of the above embodiments, is a single domain antibody comprising the sequence of SEQ ID NO: 28

In some embodiments, the MSLN binding protein, according to any one of the above embodiments, is a humanized single domain antibody comprising the sequence of SEQ ID NO: 30. In some embodiments, the MSLN binding protein, according to any one of the above embodiments, is a humanized single domain antibody comprising the sequence of SEQ ID NO: 31. In some embodiments, the MSLN binding protein, according to any one of the above embodiments, is a humanized single domain antibody comprising the sequence of SEQ ID NO: 32. In some embodiments, the MSLN binding protein, according to any one of the above embodiments, is a humanized single domain antibody comprising the sequence of SEQ ID NO: 33. In some embodiments, the MSLN binding protein, according to any one of the above embodiments, is a humanized single domain antibody comprising the sequence of SEQ ID NO: 34. In some embodiments, the MSLN binding protein, according to any one of the above embodiments, is a humanized single domain antibody comprising the sequence of SEQ ID NO: 35. In some embodiments, the MSLN binding protein, according to any one of the above embodiments, is a humanized single domain antibody comprising the sequence of SEQ ID NO: 36. In some embodiments, the MSLN binding protein, according to any one of the above embodiments, is a humanized single domain antibody comprising the sequence of SEQ ID NO: 37. In some embodiments, the MSLN binding protein, according to any one of the above embodiments, is a humanized single domain antibody comprising the sequence of SEQ ID NO: 38. In some embodiments, the MSLN binding protein, according to any one of the above embodiments, is a humanized single domain antibody comprising the sequence of SEQ ID NO: 39. In some embodiments, the MSLN binding protein, according to any one of the above embodiments, is a humanized single domain antibody comprising the sequence of SEQ ID NO: 40. In some embodiments, the MSLN binding protein, according to any one of the above embodiments, is a humanized single domain antibody comprising the sequence of SEQ ID NO: 58. In some embodiments, the MSLN binding protein, according to any one of the above embodiments, is a humanized single domain antibody comprising the sequence of SEQ ID NO: 60. In some embodiments, the MSLN binding protein, according to any one of the above embodiments, is a humanized single domain antibody comprising the sequence of SEQ ID NO: 61. In some embodiments, the MSLN binding protein, according to any one of the above embodiments, is a humanized single domain antibody comprising the sequence of SEQ ID NO: 62.

In some embodiments, the MSLN binding protein is cross-reactive with human and cynomolgus mesothelin. In some embodiments, the MSLN binding protein is specific for human mesothelin. In certain embodiments, the MSLN binding protein disclosed herein binds to human mesothelin with a human Kd (hKd). In certain embodiments, the MSLN binding protein disclosed herein binds to cynomolgus mesothelin with a cyno Kd (cKd). In certain embodiments, the MSLN binding protein disclosed herein binds to both cynomolgus mesothelin and a human mesothelin, with a cyno Kd (cKd) and a human Kd, respectively (hKd). In some embodiments, the MSLN binding protein binds to human and cynomolgus mesothelin with comparable binding affinities (i.e., hKd and cKd values do not differ by more than ±10%). In some embodiments, the hKd and the cKd range from about 0.1 nM to about 500 nM. In some embodiments, the hKd and the cKd range from about 0.1 nM to about 450 nM. In some embodiments, the hKd and the cKd range from about 0.1 nM to about 400 nM. In some embodiments, the hKd and the cKd range from about 0.1 nM to about 350 nM. In some embodiments, the hKd and the cKd range from about 0.1 nM to about 300 nM. In some embodiments, the hKd and the cKd range from about 0.1 nM to about 250 nM. In some embodiments, the hKd and the cKd range from about 0.1 nM to about 200 nM. In some embodiments, the hKd and the cKd range from about 0.1 nM to about 150 nM. In some embodiments, the hKd and the cKd range from about 0.1 nM to about 100 nM. In some embodiments, the hKd and the cKd range from about 0.1 nM to about 90 nM. In some embodiments, the hKd and the cKd range from about 0.2 nM to about 80 nM. In some embodiments, the hKd and the cKd range from about 0.3 nM to about 70 nM. In some embodiments, the hKd and the cKd range from about 0.4 nM to about 50 nM. In some embodiments, the hKd and the cKd range from about 0.5 nM to about 30 nM. In some embodiments, the hKd and the cKd range from about 0.6 nM to about 10 nM. In some embodiments, the hKd and the cKd range from about 0.7 nM to about 8 nM. In some embodiments, the hKd and the cKd range from about 0.8 nM to about 6 nM. In some embodiments, the hKd and the cKd range from about 0.9 nM to about 4 nM. In some embodiments, the hKd and the cKd range from about 1 nM to about 2 nM.

In some embodiments, any of the foregoing MSLN binding proteins (e.g., anti-MSLN single domain antibodies of SEQ ID NOs: 1-40, and 58) are affinity peptide tagged for ease of purification. In some embodiments, the affinity peptide tag is six consecutive histidine residues, also referred to as 6X-his (SEQ ID NO: 336).

In certain embodiments, the MSLN binding proteins according to the present disclosure may be incorporated into MSLN targeting trispecific proteins. In some examples, the trispecific binding protein comprises a CD3 binding domain, a human serum albumin (HSA) binding domain and an anti-MSLN binding domain according to the present disclosure. In some instances, the trispecific binding protein comprises the domains described above in the following orientation: MSLN-HSA-CD3.

In certain embodiments, the MSLN binding proteins of the present disclosure preferentially bind membrane bound mesothelin over soluble mesothelin. Membrane bound mesothelin refers to the presence of mesothelin in or on the cell membrane surface of a cell that expresses mesothelin. Soluble mesothelin refers to mesothelin that is no longer on in or on the cell membrane surface of a cell that expresses or expressed mesothelin. In certain instances, the soluble mesothelin is present in the blood and/or lymphatic circulation in a subject. In one embodiment, the MSLN binding proteins bind membrane-bound mesothelin at least 5 fold, 10 fold, 15 fold, 20 fold, 25 fold, 30 fold, 40 fold, 50 fold, 100 fold, 500 fold, or 1000 fold greater than soluble mesothelin. In one embodiment, the antigen binding proteins of the present disclosure preferentially bind membrane-bound mesothelin 30 fold greater than soluble mesothelin. Determining the preferential binding of an antigen binding protein to membrane bound MSLN over soluble MSLN can be readily determined using assays well known in the art.

Integration into Chimeric Antigen Receptors (CAR)

The MSLN binding proteins of the present disclosure, e.g., an anti-MSLN single domain antibody, can, in certain examples, be incorporated into a chimeric antigen receptor (CAR). An engineered immune effector cell, e.g., a T cell or NK cell, can be used to express a CAR that includes an anti-MSLN single domain antibody as described herein. In one embodiments, the CAR including an anti-MSLN single domain antibody as described herein is connected to a transmembrane domain via a hinge region, and further a costimulatory domain, e.g., a functional signaling domain obtained from OX40, CD27, CD28, CDS, ICAM-1, LFA-1 (CD11a/CD18), ICOS (CD278), or 4-1BB. In some embodiments, the CAR further comprises a sequence encoding a intracellular signaling domain, such as 4-1BB and/or CD3 zeta.

### Tumor Growth Reduction Properties

In certain embodiments, the MSLN binding proteins of the disclosure reduces the growth of tumor cells in vivo when administered to a subject who has tumor cells that express mesothelin. Measurement of the reduction of the growth of tumor cells can be determined by multiple different methodologies well known in the art. Nonlimiting examples include direct measurement of tumor dimension, measurement of excised tumor mass and comparison to control subjects, measurement via imaging techniques (e.g., CT or MRI) that may or may not use isotopes or luminescent molecules (e.g., luciferase) for enhanced analysis, and the like. In specific embodiments, administration of the antigen binding agents of the disclosure results in a reduction of in vivo growth of tumor cells as compared to a control antigen binding agent by at least about 10%, 20%, 30%, 40%, 50%, 60%, 70%, 80%, 90% or 100%, with an about 100% reduction in tumor growth indicating a complete response and disappearance of the tumor. In further embodiments, administration of the antigen binding agents of the disclosure results in a reduction of in vivo growth of tumor cells as compared to a control antigen binding agent by about 50-100%, about 75-100% or about 90-100%. In further embodiments, administration of the antigen binding agents of the disclosure results in a reduction of in vivo growth of tumor cells as compared to a control antigen binding agent by about 50-60%, about 60-70%, about 70-80%, about 80-90%, or about 90-100%.

### MSLN Binding Protein Modifications

The MSLN binding proteins described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence to block an immunogenic domain and/or for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in the MSLN binding proteins described herein, including the peptide backbone, the amino acid side-chains, and the amino or carboxyl termini. Certain common peptide modifications that are useful for modification of MSLN binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

### Polynucleotides Encoding MSLN Binding Proteins

Also provided, in some embodiments, are polynucleotide molecules encoding a MSLN binding protein as described herein. In some embodiments, the polynucleotide molecules are provided as DNA constructs. In other embodiments, the polynucleotide molecules are provided as messenger RNA transcripts.

The polynucleotide molecules are constructed by known methods such as by combining the genes encoding the anti-MSLN binding protein, operably linked to a suitable promoter, and optionally a suitable transcription terminator, and expressing it in bacteria or other appropriate expression system such as, for example CHO cells.

In some embodiments, the polynucleotide is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described MSLN binding protein. Examples of expression vectors for expression in E. coli are pSKK (Le Gall et al., J Immunol Methods. (2004) 285(1):111-27), pcDNA5 (Invitrogen) for expression in mammalian cells, PICHIAPINK™ Yeast Expression Systems (Invitrogen), BACUVANCE™ Baculovirus Expression System (GenScript).

Thus, the MSLN binding proteins as described herein, in some embodiments, are produced by introducing a vector encoding the protein as described above into a host cell and culturing said host cell under conditions whereby the protein domains are expressed, may be isolated and, optionally, further purified.

### Pharmaceutical Compositions

Also provided, in some embodiments, are pharmaceutical compositions comprising a MSLN binding protein described herein, a vector comprising the polynucleotide encoding the polypeptide of the MSLN binding proteins or a host cell transformed by this vector and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents. A further embodiment provides one or more of the above described binding proteins, such as anti-MSLN single domain antibodies or antigen-binding fragments thereof packaged in lyophilized form, or packaged in an aqueous medium.

In some embodiments of the pharmaceutical compositions, the MSLN binding protein described herein is encapsulated in nanoparticles. In some embodiments, the nanoparticles are fullerenes, liquid crystals, liposome, quantum dots, superparamagnetic nanoparticles, dendrimers, or nanorods. In other embodiments of the pharmaceutical compositions, the MSLN binding protein is attached to liposomes. In some instances, the MSLN binding protein is conjugated to the surface of liposomes. In some instances, the MSLN binding protein is encapsulated within the shell of a liposome. In some instances, the liposome is a cationic liposome.

The MSLN binding proteins described herein are contemplated for use as a medicament. Administration is effected by different ways, e.g., by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

In some embodiments, the MSLN binders of this disclosure are administered at a dosage of up to 10 mg/kg at a frequency of once a week. In some cases, the dosage ranges from about 1 ng/kg to about 10 mg/kg. In some embodiments, the dose is from about 1 ng/kg to about 10 ng/kg, about 5 ng/kg to about 15 ng/kg, about 12 ng/kg to about 20 ng/kg, about 18 ng/kg to about 30 ng/kg, about 25 ng/kg to about 50 ng/kg, about 35 ng/kg to about 60 ng/kg, about 45 ng/kg to about 70 ng/kg, about 65 ng/kg to about 85 ng/kg, about 80 ng/kg to about 1 μg/kg, about 0.5 μg/kg to about 5 μg/kg, about 2 μg/kg to about 10 μg/kg, about 7 μg/kg to about 15 μg/kg, about 12 μg/kg to about 25 μg/kg, about 20 μg/kg to about 50 μg/kg, about 35 μg/kg to about 70 μg/kg, about 45 μg/kg to about 80 μg/kg, about 65 μg/kg to about 90 μg/kg, about 85 μg/kg to about 0.1 mg/kg, about 0.095 mg/kg to about 10 mg/kg. In some cases, the dosage is about 0.1 mg/kg to about 0.2 mg/kg; about 0.25 mg/kg to about 0.5 mg/kg, about 0.45 mg/kg to about 1 mg/kg, about 0.75 mg/kg to about 3 mg/kg, about 2.5 mg/kg to about 4 mg/kg, about 3.5 mg/kg to about 5 mg/kg, about 4.5 mg/kg to about 6 mg/kg, about 5.5 mg/kg to about 7 mg/kg, about 6.5 mg/kg to about 8 mg/kg, about 7.5 mg/kg to about 9 mg/kg, or about 8.5 mg/kg to about 10 mg/kg. The frequency of administration, in some embodiments, is about less than daily, every other day, less than once a day, twice a week, weekly, once in 7 days, once in two weeks, once in two weeks, once in three weeks, once in four weeks, or once a month. In some cases, the frequency of administration is weekly. In some cases, the frequency of administration is weekly and the dosage is up to 10 mg/kg. In some cases, duration of administration is from about 1 day to about 4 weeks or longer.

### Methods of Treatment

Also provided herein, in some embodiments, are methods and uses for stimulating the immune system of an individual in need thereof comprising administration of a MSLN binding protein as described herein. In some instances, the administration of a MSLN binding protein described herein induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or fibrotic tissue.

Also provided herein are methods and uses for a treatment of a disease, disorder or condition associated with a target antigen comprising administering to an individual in need thereof a MSLN binding protein or a multi specific binding protein comprising the MSLN binding protein described herein. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In one embodiment, the disease, disorder or condition associated with a target antigen is cancer. Cancers that can be treated, prevented, or managed by the MSLN binding proteins of the present disclosure, and methods of using them, include but are not limited to cancers of an epithelial cell origin. Examples of such cancers include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The MSLN binding proteins of the disclosure are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the disclosure. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the skin, lung, colon, breast, prostate, bladder, kidney, pancreas, ovary, or uterus. In other specific embodiments, sarcoma, melanoma, or leukemia is treated or prevented.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, the MSLN binding proteins as described herein are administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (γ-rays, X-rays, and/or the directed delivery of radioisotopes, microwaves, UV radiation and the like), gene therapies (e.g., antisense, retroviral therapy and the like) and other immunotherapies. In some embodiments, an MSLN binding protein as described herein is administered in combination with anti-diarrheal agents, anti-emetic agents, analgesics, opioids and/or non-steroidal anti-inflammatory agents. In some embodiments, an MSLN binding protein as described herein is administered in combination with anti-cancer agents. Nonlimiting examples of anti-cancer agents that can be used in the various embodiments of the disclosure, including pharmaceutical compositions and dosage forms and kits of the disclosure, include: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1 interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinzolidine sulfate; vinzolidine sulfate; vorozole; zeniplatin; zinostatin; zorubicin hydrochloride. Other examples of anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; HMG-CoA reductase inhibitor (such as but not limited to, Lovastatin, Pravastatin, Fluvastatin, Statin, Simvastatin, and Atorvastatin); loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; Vitaxin®; vorozole; zanoterone; zeniplatin; zilascorb; and zinostatin stimalamer. Additional anti-cancer drugs are 5-fluorouracil and leucovorin. These two agents are particularly useful when used in methods employing thalidomide and a topoisomerase inhibitor. In some embodiments, the anti-MSLN single domain binding protein of the present disclosure is used in combination with gemcitabine.

In some embodiments, an MSLN binding proteins as described herein is administered before, during, or after surgery.

### Methods of Detection of Mesothelin Expression and Diagnosis of Mesothelin Associated Cancer

According to another embodiment of the disclosure, kits for detecting expression of mesothelin in vitro or in vivo are provided. The kits include the foregoing MSLN binding proteins (e.g., a labeled anti-MSLN single domain antibody or antigen binding fragments thereof), and one or more compounds for detecting the label. In some embodiments, the label is selected from the group consisting of a fluorescent label, an enzyme label, a radioactive label, a nuclear magnetic resonance active label, a luminescent label, and a chromophore label.

In some cases, mesothelin expression is detected in a biological sample. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. A biological sample is typically obtained from a mammal, such as a human or non-human primate.

In one embodiment, provided is a method of determining if a subject has cancer by contacting a sample from the subject with an anti-MSLN single domain antibody as disclosed herein; and detecting binding of the single domain antibody to the sample. An increase in binding of the antibody to the sample as compared to binding of the antibody to a control sample identifies the subject as having cancer.

In another embodiment, provided is a method of confirming a diagnosis of cancer in a subject by contacting a sample from a subject diagnosed with cancer with an anti-MSLN single domain antibody as disclosed herein; and detecting binding of the antibody to the sample. An increase in binding of the antibody to the sample as compared to binding of the antibody to a control sample confirms the diagnosis of cancer in the subject.

In some examples of the disclosed methods, the single domain antibody is directly labeled.

In some examples, the methods further include contacting a second antibody that specifically binds the single domain antibody with the sample; and detecting the binding of the second antibody. An increase in binding of the second antibody to the sample as compared to binding of the second antibody to a control sample detects cancer in the subject or confirms the diagnosis of cancer in the subject.

In some cases, the cancer is mesothelioma, prostate cancer, lung cancer, stomach cancer, squamous cell carcinoma, pancreatic cancer, cholangiocarcinoma, triple negative breast cancer or ovarian cancer, or any other type of cancer that expresses mesothelin.

In some examples, the control sample is a sample from a subject without cancer. In particular examples, the sample is a blood or tissue sample.

In some cases, the antibody that binds (for example specifically binds) mesothelin is directly labeled with a detectable label. In another embodiment, the antibody that binds (for example, specifically binds) mesothelin (the first antibody) is unlabeled and a second antibody or other molecule that can bind the antibody that specifically binds mesothelin is labeled. A second antibody is chosen such that it is able to specifically bind the specific species and class of the first antibody. For example, if the first antibody is a llama IgG, then the secondary antibody may be an anti-llama-IgG. Other molecules that can bind to antibodies include, without limitation, Protein A and Protein G, both of which are available commercially. Suitable labels for the antibody or secondary antibody are described above, and include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include 125I, 131I, 35S or 3H.

In an alternative embodiment, mesothelin can be assayed in a biological sample by a competition immunoassay utilizing mesothelin standards labeled with a detectable substance and an unlabeled antibody that specifically binds mesothelin. In this assay, the biological sample, the labeled mesothelin standards and the antibody that specifically bind mesothelin are combined and the amount of labeled mesothelin standard bound to the unlabeled antibody is determined. The amount of mesothelin in the biological sample is inversely proportional to the amount of labeled mesothelin standard bound to the antibody that specifically binds mesothelin.

The immunoassays and method disclosed herein can be used for a number of purposes. In one embodiment, the antibody that specifically binds mesothelin may be used to detect the production of mesothelin in cells in cell culture. In another embodiment, the antibody can be used to detect the amount of mesothelin in a biological sample, such as a tissue sample, or a blood or serum sample. In some examples, the mesothelin is cell-surface mesothelin. In other examples, the mesothelin is soluble mesothelin (e.g., mesothelin in a cell culture supernatant or soluble mesothelin in a body fluid sample, such as a blood or serum sample).

In one embodiment, a kit is provided for detecting mesothelin in a biological sample, such as a blood sample or tissue sample. For example, to confirm a cancer diagnosis in a subject, a biopsy can be performed to obtain a tissue sample for histological examination. Alternatively, a blood sample can be obtained to detect the presence of soluble mesothelin protein or fragment. Kits for detecting a polypeptide will typically comprise a single domain antibody, according to the present disclosure, that specifically binds mesothelin. In some embodiments, an antibody fragment, such as an scFv fragment, a VH domain, or a Fab is included in the kit. In a further embodiment, the antibody is labeled (for example, with a fluorescent, radioactive, or an enzymatic label).

In one embodiment, a kit includes instructional materials disclosing means of use of an antibody that binds mesothelin. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment, the diagnostic kit comprises an immunoassay. Although the details of the immunoassays may vary with the particular format employed, the method of detecting mesothelin in a biological sample generally includes the steps of contacting the biological sample with an antibody which specifically reacts, under immunologically reactive conditions, to a mesothelin polypeptide. The antibody is allowed to specifically bind under immunologically reactive conditions to form an immune complex, and the presence of the immune complex (bound antibody) is detected directly or indirectly.

Methods of determining the presence or absence of a cell surface marker are well known in the art. For example, the antibodies can be conjugated to other compounds including, but not limited to, enzymes, magnetic beads, colloidal magnetic beads, haptens, fluorochromes, metal compounds, radioactive compounds or drugs. The antibodies can also be utilized in immunoassays such as but not limited to radioimmunoassays (RIAs), ELISA, or immunohistochemical assays. The antibodies can also be used for fluorescence activated cell sorting (FACS). FACS employs a plurality of color channels, low angle and obtuse light-scattering detection channels, and impedance channels, among other more sophisticated levels of detection, to separate or sort cells (see U.S. Pat. No. 5,061,620). Any of the single domain antibodies that bind mesothelin, as disclosed herein, can be used in these assays. Thus, the antibodies can be used in a conventional immunoassay, including, without limitation, an ELISA, an RIA, FACS, tissue immunohistochemistry, Western blot or imunoprecipitation.

### EXAMPLES

The examples below further illustrate the described embodiments without limiting the scope of the invention.

### Example 1: Ability of an Exemplar Anti-MSLN Single Domain Antibody of the Present Disclosure to Mediate T Cell Killing of Cancer Cells Expressing Mesothelin

An exemplar anti-MSLN single domain antibody sequence is transfected into Expi293 cells (Invitrogen). The amount of the exemplar anti-MSLN antibody in the conditioned media from the transfected Expi293 cells is quantitated using an Octet instrument with Protein A tips and using a control anti-MSLN antibody as a standard curve.

Titrations of conditioned media is added to TDCC assays (T cell Dependent Cell Cytotoxicity assays) to assess whether the anti-MSLN single domain antibody is capable of forming a synapse between T cells and a mesothelin expressing ovarian cancer cell line, OVCAR8. Viability of the OVCAR8 cells is measured after 48 hours. It is seen that the exemplar anti-MSLN single domain antibody mediates T cell killing.

Furthermore, it is seen that the TDCC activity of the exemplar anti-MSLN single domain antibody is specific to mesothelin expressing cells, because the exemplar antibody does not mediate T cell killing of LNCaP cells, which do not express mesothelin.

### Example 2: Methods to Assess Binding and Cytotoxic Activities of Several MSLN Targeting Trispecific Antigen Binding Proteins Containing a MSLN Binding Domain According to the Present Disclosure

Protein Production

Sequences of MSLN targeting trispecific molecules, containing a MSLN binding protein according to the present disclosure, were cloned into mammalian expression vector pCDNA 3.4 (Invitrogen) preceded by a leader sequence and followed by a 6× Histidine Tag (SEQ ID NO: 336). Expi293F cells (Life Technologies A14527) were maintained in suspension in Optimum Growth Flasks (Thomson) between 0.2 to 8×1e6 cells/mL in Expi 293 media. Purified plasmid DNA was transfected into Expi293 cells in accordance with Expi293 Expression System Kit (Life Technologies, A14635) protocols, and maintained for 4-6 days post transfection. The amount of the exemplary trispecific proteins being tested, in the conditioned media, from the transfected Expi293 cells was quantitated using an Octet instrument with Protein A tips and using a control trispecific protein for a standard curve.

Cytotoxicity Assays

A human T-cell dependent cellular cytotoxicity (TDCC) assay was used to measure the ability of T cell engagers, including trispecific molecules, to direct T cells to kill tumor cells (Nazarian et al. 2015. J Biomol Screen. 20:519-27). In this assay, T cells and target cancer cell line cells are mixed together at a 10:1 ratio in a 384 wells plate, and varying amounts of the trispecific proteins being tested are added. The tumor cell lines are engineered to express luciferase protein. After 48 hours, to quantitate the remaining viable tumor cells, Steady-Glo® Luminescent Assay (Promega) was used.

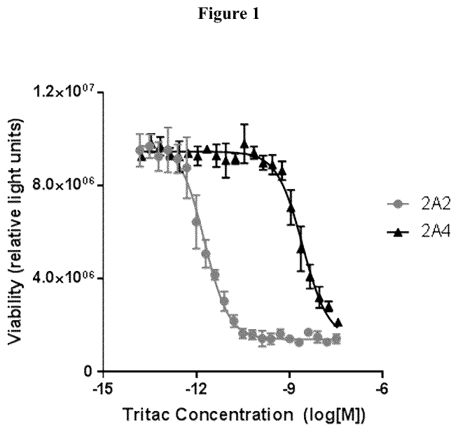

In the instant study, titrations of conditioned media was added to TDCC assays (T cell Dependent Cell Cytotoxicity assays) to assess whether the anti-MSLN single domain antibody was capable of forming a synapse between T cells and a mesothelin expressing ovarian cancer cell line, OVCAR8. Viability of the OVCAR8 cells was measured after 48 hours. It was seen that the trispecific proteins mediated T cell killing. FIG. 1 shows an example cell viability assay with test trispecific proteins 2A2 and 2A4. The EC₅₀ for the TDCC activity of several other test trispecific proteins are listed below in Table 1.

TABLE 1
TDCC Activity of MSLN targeting trispecific proteins containing
a MSLB binding protein according to the present disclosure

| Anti-MSLN TriTAC | Average EC50 [M] |
|---|---|
| 2A2 | 1.6E−12 |
| 2A4 | 1.9E−09 |
| 11F3 | 2.2E−12 |
| 5D4 | 1.0E−09 |
| 9H2 | 1.1E−12 |
| 5C2 | 1.5E−12 |
| 5G2 | 3.6E−09 |
| 10B3 | 1.4E−12 |
| 2F4 | 7.3E−13 |
| 2C2 | 9.5E−09 |
| 5F2 | 5.3E−12 |
| 7C4 | 1.0E−08 |
| 7F1 | 2.4E−12 |
| 5D2 | 1.4E−11 |
| 6H2 | 2.0E−09 |
| 2D1 | 5.2E−11 |
| 12C2 | 8.0E−13 |
| 3F2 | 2.4E−08 |
| 1H2 | 2.5E−08 |
| 6F3 | 8.2E−10 |
| 2A1 | 1.2E−09 |
| 3G1 | 4.0E−09 |
| 12D1 | 1.1E−09 |
| 5H1 | 5.9E−12 |
| 4A2 | 1.7E−09 |
| 3B4 | 1.8E−12 |
| 7H2 | 5.5E−12 |
| 9F3 | >1E−7 |
| 9B1 | >1E−7 |

Furthermore, it was observed that the TDCC activity of the MSLN targeting trispecific proteins being tested was specific to mesothelin expressing cells, because the trispecific proteins being tested did not mediate T cell killing of LNCaP cells, which do not express mesothelin. The trispecific proteins 2A2, 11F3, 9H2, 5C2, 10B3, 2F4, 5F2, 7F1, 2F4, 5H1, 3B4, and 7H2, in particular did not show any TDCC activity with the LnCaP cells.

### Example 3: ADCC Activity of an Exemplar Anti-MSLN Single Domain Antibody of the Present Disclosure

This study is directed to determining the ability of an exemplary anti-MSLN single domain antibody of the present disclosure to mediate ADCC as compared to a comparator llama anti-MSLN antibody which does not have sequence modifications or substitutions as the exemplary antibody of the disclosure. Both antibodies are expressed as multidomain proteins which include additional immunoglobulin domains.

Materials

Donors are leukophoresed, and NK cells are isolated from the leukopack by the Cell Purification Group using the Milteni AutoMacs Pro negative selection system. NK cells are held overnight at 4° C. on a rocker, then washed, counted and resuspended at 4×10⁶ cells/mL in complete RPMI for use in the ADCC assay.

Targets: Tumor cell targets are selected based on mesothelin expression. Targets are washed and counted. 6×10⁶ targets are resuspended in complete RPMI and labeled in a final concentration of 10 μM calcein (Sigma #C1359-00UL CALCEIN AM 4 MINI IN ANHYDROUS DMSO) for 40 minutes at 37° C., 5% CO2. Cells are washed twice in PBS, resuspended in complete RPMI and incubated at 37° C., 5% CO2 for 2 hrs. After labeling, target cells are washed, recounted and resuspended at 0.2×106 cells/mL in complete RPMI for use in the ADCC assay.

Methods

The ADCC assay is performed in a 96 well round bottom tissue culture plate (Corning 3799). The test proteins are titrated from 20 μg/mL to 0.0002 μg/mL by carrying 10 μL in 1000 μL of complete RPMI containing 10% FCS (a 1:10 dilution). Calcein labeled targets are added, 50 μL to contain 10,000 cells. Target cells and various concentrations of the multidomain proteins containing either the exemplar anti-MSLN single domain antibody or the comparator antibody are incubated for 40 minutes at 4° C., then NK cell effectors added, 50 μL to contain 100,000 cells (10:1 E:T ratio). Cultures are incubated for 4 hrs at 37° C. then supernatants pulled and assayed for calcein release by measuring fluorescence at 485-535 nm on a Wallac Victor II 1420 Multilable HTS counter. 100% lysis values are determined by lysing six wells of labeled targets with Igepal 630 detergent (3 μL per well) and spontaneous lysis values determined by measuring the fluorescence in supernatants from targets alone.

Statistical Analysis

Percent (%) specific lysis is defined as (sample fluorescence)−(spontaneous lysis fluorescence)/(100% lysis−spontaneous lysis fluorescence). Spontaneous lysis is determined by wells containing only targets and 100% lysis is determined by wells where targets are lysed with IGEPAL CA 630 detergent. Raw data is entered in an Excel spreadsheet with embedded formulae to calculate % specific lysis and resultant values transferred to graphic program (GraphPad Prism) where the data is transformed in a curve fit graph Subsequent analyses (linear regression calculations) are done in GraphPad to generate EC₅₀ values.

Results and Discussion

Effector NK cells in wells incubated with the multidomain protein containing the comparator anti-MSLN antibody are unable to mediate killing of the calcein-labeled target cells while effectors in wells with the multidomain protein containing the exemplar anti-MSLN single domain antibody of the present disclosure are, as measured by specific Lytic activity (% specific lysis) able to mediate antibody dependent cellular cytotoxicity.

Conclusions

The exemplary anti-MSLN single domain antibody of the present disclosure mediates a significantly higher level of killing, of target cells expressing mesothelin, than the comparator llama anti-MSLN single domain antibody with no sequence substitutions, modification, or humanization.

### Example 4: CDC Activity of an Exemplar Anti-MSLN Single Domain Antibody of the Present Disclosure

To evaluate the anti-tumor activity of exemplar anti-MSLN single domain antibody, according to the present disclosure, against cancer cells, the cytotoxic activity in A431/H9 and NCI-H226 cell models in the presence of human serum as a source of complement is tested. The exemplar anti-MSLN single domain antibody is expressed as a multidomain protein containing additional immunoglobulin domains. It is seen that the multidomain protein containing the exemplar anti-MSLN single domain antibody of the present disclosure exerts potent CDC activity by killing about 40% of A431/H9 and more than 30% of NCI-H226 mesothelioma cell lines, and shows no activity on the mesothelin-negative A431 cell line. A comparator llama anti-MSLN antibody, which does not have sequence modifications or substitutions as the exemplary antibody of the disclosure, shows no activity at the same concentrations.

In order to analyze the role of complement in the anti-tumor activity of the exemplar anti-MSLN single domain antibody, flow cytometry is used to determine C1q binding to cancer cells reacted with anti-mesothelin human mAbs following a well-established protocol for characterization of rituximab, ofatumumab and other anti-CD20 therapeutic mAbs (Pawluczkowycz et al., J Immunol 183:749-758, 2009; Li et al., Cancer Res 68:2400-2408, 2008). It has previously been shown that like MORAb-009, the HN1 human mAb specific for Region I of cell surface mesothelin (far from the cell surface), did not exhibit any CDC activity against mesothelin-expressing cancer cells (Ho et al., Int J Cancer 128:2020-2030, 2011).

However, it is seen that the C1q complement binds to A431/H9 or NCI-H226 cells in the presence of exemplar anti-MSLN single domain antibody. In contrast, no C1q binding is found in the presence of the comparator llama anti-MSLN antibody. Moreover, the binding of C1q to cancer cells is associated with the cell binding of exemplar anti-MSLN single domain antibody in a dose-response manner. These results demonstrate that the exemplar anti-MSLN single domain antibody demonstrates improved CDC activity relative to the comparator llama anti-MSLN antibody.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 5: MSLN Targeting Trispecific Antigen Binding Protein Containing a MSLN Binding Domain (MH6T) According to the Present Disclosure Directs T Cells to Kill MSLN Expressing Ovarian Cancer Cells

A human T-cell dependent cellular cytotoxicity (TDCC) assay was used to measure the ability of T cell engagers, including trispecific molecules, to direct T cells to kill tumor cells (Nazarian et al. 2015. J Biomol Screen. 20:519-27). The Caov3 cells used in this assay were engineered to express luciferase. T cells from 5 different healthy donors (donor 02, donor 86, donor 41, donor 81, and donor 34) and target cancer cells Caov3 were mixed together and varying amounts of an MSLN targeting trispecific antigen binding protein containing the MSLN binding domain (1\41-16T) (SEQ ID NO: 58) was added and the mixture was incubated for 48 hours at 37° C. Caov3 cells and T cells were also incubated for 48 hours at 37° C. with a control trispecific molecule, GFP TriTAC (SEQ ID NO: 59), which targets GFP. After 48 hours, the remaining viable tumor cells were quantified by a luminescence assay.

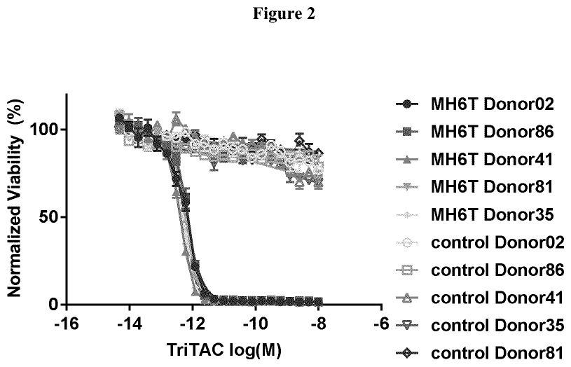

It was observed that the MSLN targeting trispecific antigen binding protein containing the MSLN binding domain (MH6T) was able to direct the T cells from all 5 healthy donors to kill the target cancer cells Caov3 (as shown in FIG. 2), whereas the control GFP TriTAC molecule was not able to direct the T cells from any of the 5 healthy donors to kill the Caov3 cells (also shown in FIG. 2).

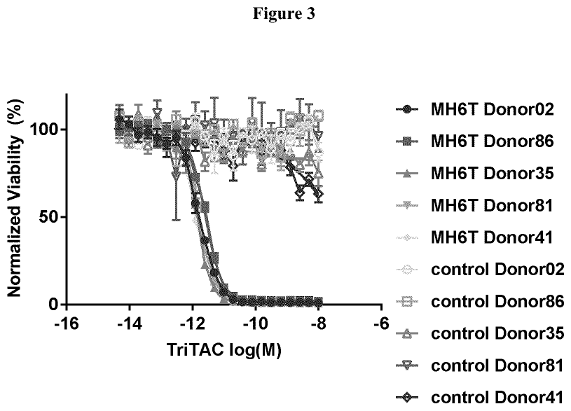

A further assay, using the same protocol as described above, was carried out using OVCAR3 cells. It was observed that the MSLN targeting trispecific antigen binding protein containing the MSLN binding domain (MH6T) was able to direct the T cells from all 5 healthy donors to kill the target cancer cells OVCAR3 (as shown in FIG. 3), whereas the control GFP TriTAC molecule was not able to direct the T cells from any of the 5 health donors to kill the OVCAR3 cells (also shown in FIG. 3).

The EC₅₀ values for killing of MSLN expressing target cells are listed below in Table II.

TABLE II
EC₅₀ values for MSLN targeting trispecific antigen binding
protein (containing the MH6T domain) directed killing
of MSLN-expressing ovarian cancer cell lines by T cells
from 5 different healthy donors. Represented graphs
of the raw data are provided in FIGS. 2 and 3.

| | EC₅₀ values (M) | | | | |
|---|---|---|---|---|---|
| | Donor02 | Donor86 | Donor41 | Donor81 | Donor35 |
| Caov3 | 6.0E−13 | 6.8E−13 | 3.9E−13 | 5.9E−13 | 4.6E−13 |
| Caov4 | 7.3E−12 | 1.1E−11 | 3.7E−12 | 4.7E−12 | 2.2E−12 |
| OVCAR3 | 1.6E−12 | 2.5E−12 | 1.4E−12 | 1.6E−12 | 1.3E−12 |
| OVCAR8 | 2.2E−12 | 3.2E−12 | 1.4E−12 | 1.9E−12 | 1.7E−12 |

### Example 6: MSLN Targeting Trispecific Antigen Binding Protein Containing a MSLN Binding Domain (MH6T) According to the Present Disclosure Directs T Cells to Kill Cells Expressing MSLN but not Cells that do not Express MSLN

In this assay, T cells from a healthy donor was incubated with target cancer cells that express MSLN (Caov3 cells, Caov4 cells, OVCAR3 cells, and OVCAR8 cells) or target cancer cells that do not express MSLN (NCI-H510A cells, MDAPCa2b cells). Each of the target cells used in this study were engineered to express luciferase. Varying amounts of an MSLN targeting trispecific antigen binding protein containing the MH6T (SEQ ID NO: 58)_domain_was added to the mixture of T cells and target cancer cells listed above. The mixture was incubated for 48 hours at 37° C. After 48 hours, the remaining viable target cancer cells were quantified using a luminescent assay.

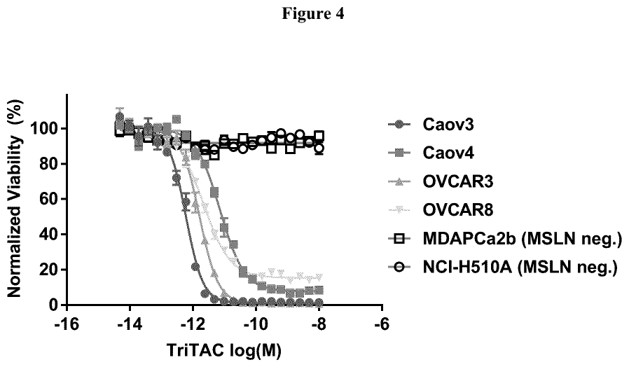

It was observed that the MSLN targeting trispecific antigen binding protein containing the MH6T domain was able to direct T cells to kill MSLN expressing target cancer cells (i.e., Caov3, Caov4, OVCAR3, and OVCAR8 cells, as shown in FIG. 4). However, the MSLN targeting trispecific antigen binding protein containing the MH6T domain was not able to direct T cells to kill MSLN non-expressing target cancer cells (MDAPCa2b and NCI-H510A cells), also shown in FIG. 4.

The EC₅₀ values for killing of MSLN expressing cancer cells are listed below in Table III.

TABLE III
EC₅₀ values for MSLN targeting trispecific antigen binding
protein (containing the MH6T domain) directed T cell
killing of MSLN-expressing cancer cell lines.

| | | EC₅₀ | MSLN sites |
|---|---|---|---|
| Tumor origin | Cell Line | (pM) | per cell |
| Ovarian | Caov3 | 0.6 | 51262 |
| | Caov4 | 7.3 | 101266 |
| | OVCAR3 | 1.6 | 40589 |
| | OVCAR8 | 2.2 | 40216 |
| | SKOV3 | 3.6 | 10617 |
| Pancreatic | Hs766T | 7.8 | 5892 |
| | CaPan2 | 3.2 | 27413 |
| | HPaFII | 15 | 17844 |
| NSCLC | NCI-H596 | 1.5 | 103769 |
| | NCI-H292 | 3.8 | 5977 |
| | NCI-H1563 | 2.6 | 17221 |
| Mesothelioma | NCI-H2052 | 8.0 | not determined |
| | NCI-H2452 | 2.3 | not determined |
| Engineered | HEK293 expressing | 0.9 | 128091 |
| (non-tumor) | human MSLN | | |
| | HEK293 293 expressing | 0.7 | 140683 |
| | cynomolgus MSLN | | |

### Example 7: MSLN Targeting Trispecific Antigen Binding Protein Containing an MSLN Binding Protein (MH6T) According to this Disclosure Directed T Cells from Cynomolgus Monkeys to Kill Human Ovarian Cancer Cell Lines

In this assay, peripheral blood mononuclear cells (PBMCs; T cells are a component of the PBMCs) from a cynomolgus monkey donor were mixed with target cancer cells that express MSLN (CaOV3 cells and OVCAR3 cells) and varying amounts of an MSLN targeting trispecific antigen binding protein (containing the MH6T domain, SEQ ID NO: 58) was added to the mixture, and incubated for 48 hours at 37° C. In parallel, a mixture of cynomolgus PBMCs and MSLN expressing cells, as above, were incubated with varying amounts of a control TriTAC molecule GFP TriTAC (SEQ ID NO: 59) that targets GFP, for 48 hours at 37° C. Target cancer cells used in this assay were engineered to express luciferase. After 48 hours, the remaining viable target cells were quantified using a luminescence assay.

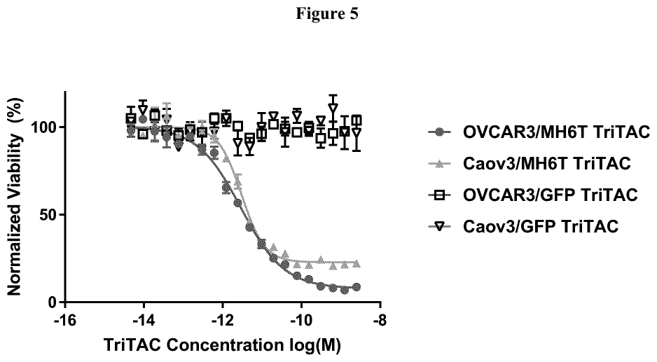

It was observed that the MSLN targeting trispecific antigen binding protein (containing the MH6T domain) was able to efficiently direct cynomolgus PBMCs to kill MSLN expressing cells (i.e., Caov3 and OVCAR), as shown in FIG. 5, whereas the control GFP TriTAC molecule was not able to direct the cynomolgus PBMCs to kill the cells (also shown in FIG. 5). The EC₅₀ values for the MSLN targeting trispecific antigen binding protein (containing the MH6T domain) was 2.9 pM for OVCAR3 cells and 3.0 pM for Caov3 cells, which were not significantly different that EC₅₀ values observed with human T cells, as shown in Table II.

### Example 8: MSLN Targeting Trispecific Antigen Binding Protein (Containing the MH6T Domain) Directed Killing of MSLN-Expressing NCI-112052 Mesothelioma Cells by T Cells in the Presence or Absence of Human Serum Albumin

The aim of this study was to assess if binding to human serum albumin (HSA) by an MSLN targeting trispecific antigen binding protein (containing the MI-16T domain; SEQ ID NO: 58) impacted the ability of the protein to direct T cells to kill MSLN-expressing cells. NCI-H2052 mesothelioma cells used in this study were engineered to express luciferase. T cells from a healthy donor and MSLN expressing cells (NCI-H2052) were mixed and varying amounts of the MSLN targeting trispecific antigen binding protein (containing the MI-16T domain) was added to the mixture. The mixture was incubated for 48 hours at 37° C., in presence or absence of HSA. A mixture of NCI-H2052 cells and T cells were also incubated for 48 hours at 37° C. with a control trispecific molecule, GFP TriTAC (SEQ ID NO: 59), which targets GFP, in presence or absence of HSA. After 48 hours, the remaining viable target cells were quantified using a luminescence assay.

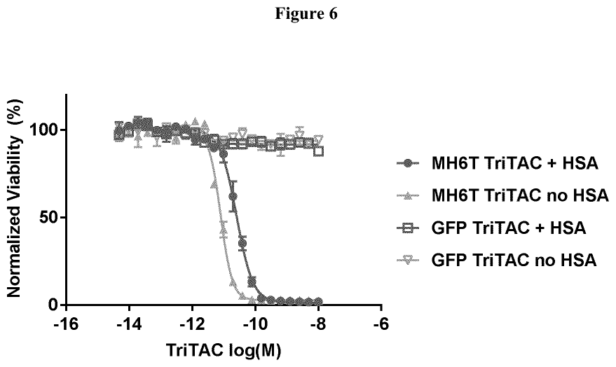

It was observed that the MSLN targeting trispecific antigen binding protein (containing the MI-16T domain) was able to efficiently direct T cells to kill NCI-H2052 cells (as shown in FIG. 6) in presence or absence of HSA, whereas the control GFP TriTAC molecule was not able to do that (also shown in FIG. 6). It was also observed that in presence of HSA, the EC₅₀ value for cell killing was increased by about 3.2 folds (as shown in Table IV).

Further TDCC assays were carried out with the MSLN targeting trispecific antigen binding protein (containing the MI-16T domain), in presence or absence of 15 mg/ml HSA, with additional MSLN-expressing cells lines and the EC₅₀ values are presented in Table IV.

TABLE IV
EC₅₀ values for MSLN targeting trispecific antigen binding protein
(containing the MH6T domain) directed killing of MSLN-expressing
cancer cells by T cells in the presence or absence of HSA

| | EC₅₀ no | EC₅₀ with | EC₅₀ |
|---|---|---|---|
| Cell line | HSA (pM) | HSA (pM) | shift (fold) |
| OVCAR8 | 2.7 | 8.7 | 3.2 |
| SKOV3 | 3.9 | 11 | 2.8 |
| NCI-H2052 | 8.0 | 26 | 3.2 |
| NCI-H24522 | 2.3 | 6.3 | 2.7 |
| Caov3 | 0.8 | 3.6 | 4.3 |
| OVCAR3 | 1.6 | 3.8 | 2.4 |

### Example 9: T Cells from 4 Different Donors Secrete TNF-α in the Presence of MSLN Targeting Trispecific Antigen Binding Protein (Containing the MH6T Domain) and MSLN-Expressing Caov4 Cells

The target cancer cells CaOv4 used in this assay were engineered to express luciferase. In this assay, T cells from 4 different healthy donors (donor 02, donor 86, donor 35, and donor 81) and Caov4 cells were mixed together and varying amounts of an MSLN targeting trispecific antigen binding protein (containing the MH6T domain; SEQ ID NO: 58) was added and the mixture was incubated for 48 hours at 37° C. Caov4 cells and T cells were also incubated for 48 hours at 37° C. with a control trispecific molecule, GFP TriTAC (SEQ ID NO: 59), which targets GFP. Conditioned medium from the TDCC assay was collected at 48 hours, before measuring the target cancer cell viability, using a luminescence assay. The concentration of TNF-α in the conditioned medium was measured using an AlphaLISA assay kit (Perkin Elmer).

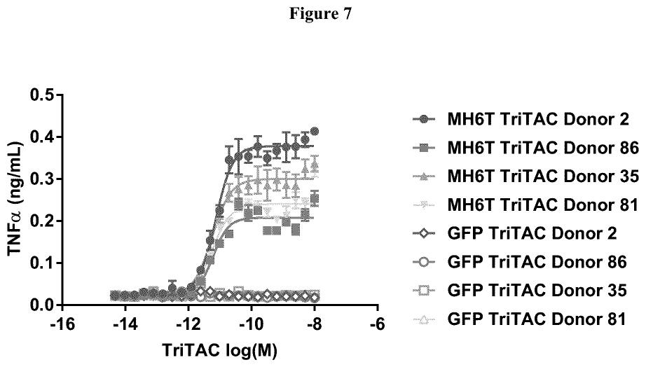

It was observed that TNF-α was secreted into the medium in presence of Caov4 cells and the MSLN targeting trispecific antigen binding protein (containing the MH6T domain) but not in presence of Caov4 cells and the control GFP TriTAC molecule, as shown in FIG. 7.

Furthermore, efficient killing was observed with T cells from all 4 healthy donors, in presence of the MSLN targeting trispecific antigen binding protein (containing the MH6T domain), but not in presence of the control GFP TriTAC molecule. TDCC assays were also set up for additional MSLN expressing cell lines (Caov3 cells, OVCAR3 cells, and OVCAR8 cells) and similar TNF-α expression was observed. The EC₅₀ values for MSLN targeting trispecific antigen binding protein (containing the MH6T domain) induced expression of TNF-α are presented in Table V. However, when the assay was carried out using cancer cells that do not express MSLN (NCI-H510A cells, or MDAPCa2b cells), no MSLN targeting trispecific antigen binding protein (containing the MH6T domain) directed secretion of TNF-α was observed (data not shown). Thus, this study demonstrated that the MSLN targeting trispecific antigen binding protein (containing the MI-16T domain) was able to activate T cells in the presence of MSLN-expressing target cancer cells.

TABLE V
EC₅₀ values for MSLN targeting trispecific antigen binding protein (containing
the MH6T domain) induced expression of TNF-α by T cells from 4
different T cell donors and 4 different MSLN-expressing cell lines

| | TNFα EC₅₀ values (M) | | | |
|---|---|---|---|---|
| | MH6T Containing | MH6T Containing | MH6T Containing | MH6T Containing |
| | Trispecific Antigen | Trispecific Antigen | Trispecific Antigen | Trispecific Antigen |
| | Binding Protein | Binding Protein | Binding Protein | Binding Protein |
| | Donor 2 | Donor 86 | Donor 35 | Donor 81 |
| Caov3 | 5.2E−12 | 5.4E−12 | 5.9E−12 | 4.9E−12 |
| Caov4 | 7.2E−12 | 6.0E−12 | 5.5E−12 | 5.5E−12 |
| OVCAR3 | 9.2E−12 | 4.0E−12 | 1.7E−11 | 8.9E−12 |
| OVCAR8 | 1.3E−11 | 9.1E−12 | 5.1E−12 | 5.0E−12 |

### Example 10: Activation of CD69 Expression on T Cells from 4 Different Donors in Presence of MSLN Targeting Trispecific Antigen Binding Protein (Containing the MH6T Domain) and MSLN-Expressing OVCAR8 Cells

The OVCAR8 cells used in this assay were engineered to express luciferase. In this assay, T cells from 4 different healthy donors (donor 02, donor 86, donor 35, and donor 81) and OVCAR8 cells were mixed together and varying amounts of the MSLN targeting trispecific antigen binding protein (containing the MI-16T domain; SEQ ID NO: 58) was added and the mixture was incubated for 48 hours at 37° C. OVCAR8 cells and T cells were also incubated for 48 hours at 37° C. with a control trispecific molecule, GFP TriTAC (SEQ ID NO: 59), which targets GFP. After 48 hours, T cells were collected, and CD69 expression on the T cells was measured by flow cytometry.

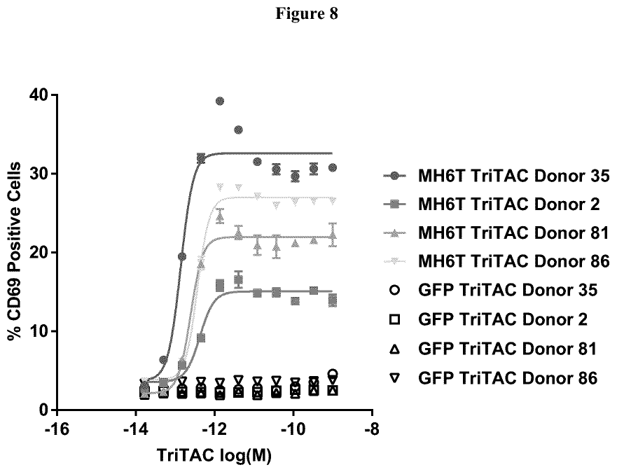

CD69 expression was detected on T cells from all 4 healthy donors in presence of OVCAR8 cells and the MSLN targeting trispecific antigen binding protein (containing the MI-16T domain) but not in presence of the negative control GFP TriTAC and OVCAR8 cells, as shown in FIG. 8. TDCC assays were also set up for additional MSLN expressing cells (Caov3 cells, OVCAR3 cells, and OVCAR8 cells) and similar CD69 expression was observed. The EC₅₀ values for MSLN targeting trispecific antigen binding protein (containing the MH6T domain) induced activation of CD69 in Caov3 cells and OVCAR8 cells are presented in Table VI.

TABLE VI
EC₅₀ values for activation of CD69 expression on T cells
from 4 different donors in presence of MSLN targeting trispecific
antigen binding protein (containing the MH6T domain) and
MSLN-expressing OVCAR8 cells or Caov3 cells.

| | Caov3 | OVCAR8 |
|---|---|---|
| EC₅₀ table | CD69 (M) | CD69 (M) |
| Donor 35 | ~1.5E−13 | 1.4E−13 |
| Donor 2 | 2.5E−13 | 4.2E−13 |
| Donor 81 | 2.5E−13 | 2.5E−13 |
| Donor 86 | 3.7E−13 | 3.7E−13 |

When the assay was carried out using cancer cells that do not express MSLN (NCI-H510A cells or MDAPCa2b cells), no MSLN targeting trispecific antigen binding protein (containing the MH6T domain) induced activation of CD69 was observed (data not shown). Thus, this study demonstrated that the MSLN targeting trispecific antigen binding protein (containing the MH6T domain) was able to activate T cells in the presence of MSLN-expressing target cancer cells.

### Example 11: Measurement of MSLN Targeting Trispecific Antigen Binding Protein (Containing the MH6T Domain) Binding to MSLN Expressing/Non-Expressing Cell Lines

For this study, certain target cancer cells that express MSLN (Caov3 cells, CaOV4 cells, OVCAR3 cells, and OVCAR8 cells) and certain cancer cells that do not express MSLN (MDAPCa2b cells, and NCI-H510A cells) were incubated with the MSLN targeting trispecific antigen binding protein (containing the MH6T domain; SEQ ID NO: 58) or a control GFP TriTAC molecule (SEQ ID NO: 59). Following incubation, the cells were washed to remove unbound MH6T or GFP TriTAC molecules and further incubated with a secondary antibody, which is able to recognize the anti-albumin domain in the TriTAC molecules, conjugated to Alexa Fluor 647. Binding of the MSLN targeting trispecific antigen binding protein (containing the MH6T domain) or that of GFP TriTAC to the MSLN expressing or MSLN non-expressing cells was measured by flow cytometry.

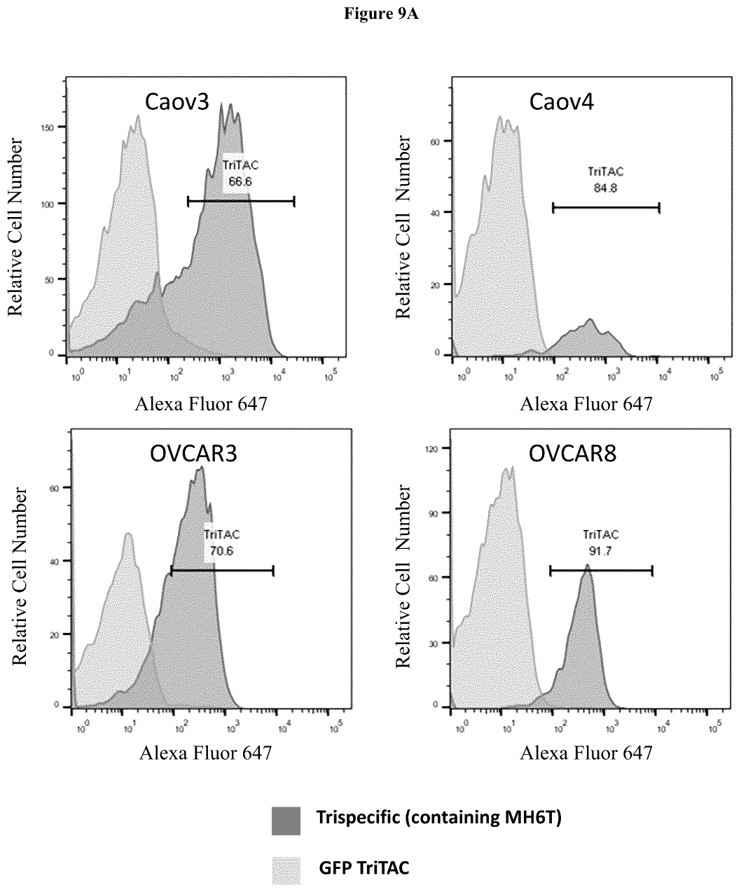

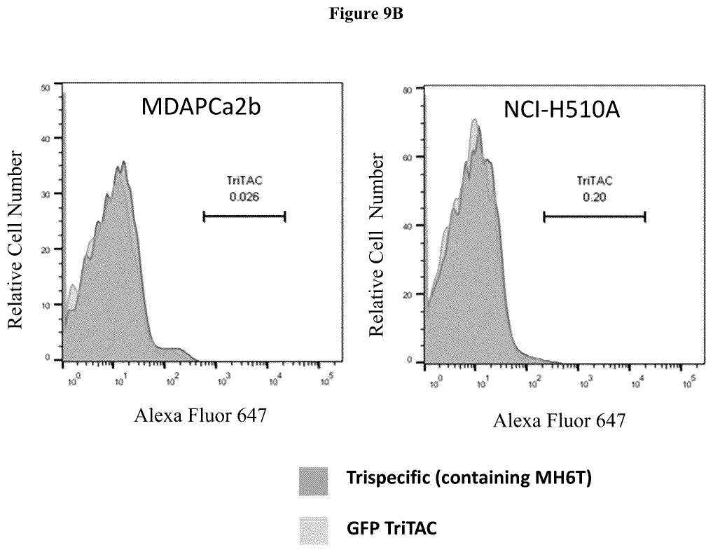

Robust binding of the MSLN targeting trispecific antigen binding protein (containing the MH6T domain) to cell lines that express MSLN (Caov3, Caov4, OVCAR3, and OVCAR8) was observed, as seen in FIG. 9A (top left panel shows binding of the MSLN targeting trispecific target antigen binding protein containing the MH6T domain to Caov3 cells; top right panel shows binding of MSLN targeting trispecific target antigen binding protein containing the MH6T domain to Caov4 cells; bottom left panel shows binding of MSLN targeting trispecific target antigen binding protein containing the MH6T domain to OVCAR3 cells; bottom right panel shows binding of MSLN targeting trispecific target antigen binding protein containing the MH6T domain to OVCAR8 cells); and as seen in FIG. 9B, no binding was observed in cell lines that do not express MSLN (left panel shows lack of binding of MSLN targeting trispecific antigen binding protein (containing the MH6T domain) to MDAPCa2b cells and the right panel shows lack of binding of MSLN targeting trispecific antigen binding protein (containing the MH6T domain) to NCI-H510A cells). Furthermore, no binding was observed when any of the cell types were incubated with the GFP TriTAC molecule, as shown in both FIGS. 9A and 9B.

### Example 12: Measurement of MSLN Targeting Trispecific Antigen Binding Protein (Containing the MH6T Domain) Binding to T Cells from Donors

For this study, T cells from 4 healthy donors were incubated with an MSLN targeting trispecific antigen binding protein (containing the MH6T domain; SEQ ID NO: 58) or a buffer, as negative control. Following incubation, the cells were washed to remove unbound MSLN targeting trispecific antigen binding protein (containing the MH6T domain) and further incubated with an Alexa Fluor 647 conjugated secondary antibody, which was able to recognize the anti-albumin domain in the MSLN targeting trispecific antigen binding protein (containing the MH6T domain). Binding was measured by flow cytometry.

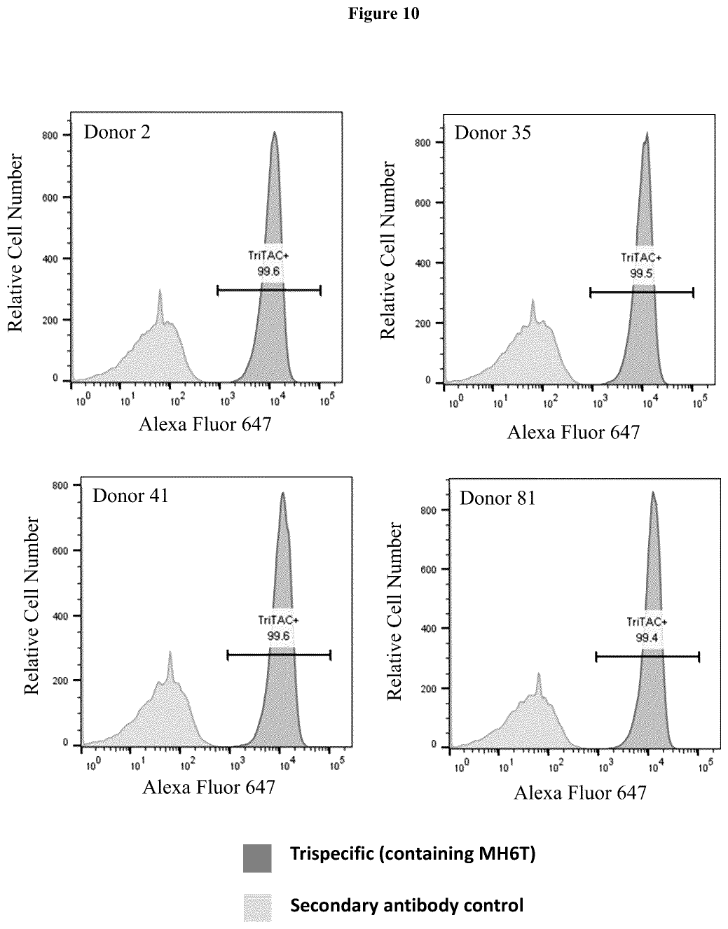

Robust binding was observed in T cells from all four donors, treated with the MSLN targeting trispecific antigen binding protein (containing the MH6T domain), as shown in FIG. 10 (top left panel shows binding of MSLN targeting trispecific antigen binding protein (containing the MH6T domain) to T cells from donor 2; top right panel shows binding of MSLN targeting trispecific antigen binding protein (containing the MH6T domain) to T cells from donor 35; bottom left panel shows binding of MSLN targeting trispecific antigen binding protein (containing the MH6T domain) to T cells from donor 41; bottom right panel shows binding of MSLN targeting trispecific antigen binding protein (containing the MH6T domain) to T cells from donor 81).

### Example 13: Inhibition of Tumor Growth in Mice Treated with MSLN Targeting Trispecific Antigen Binding Protein (Containing the MH6T Domain)

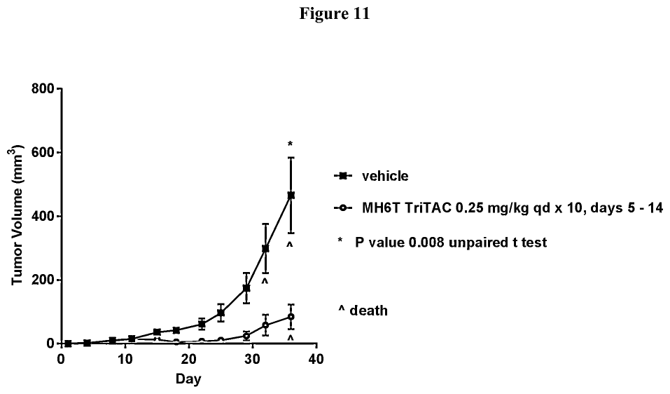

For this study, 10⁷ NCI-H292 cells and 10⁷ human PBMCs were co-implanted subcutaneously in two groups of NCG mice (8 mice per group). After 5 days, mice in one group were injected with the MSLN targeting trispecific antigen binding protein (containing the MH6T domain; SEQ ID NO: 58), daily for 10 days (days 5-14) at a dose of 0.25 mg/kg; and mice in the other group were injected with a vehicle control. Tumor volumes were measured after every few days and the study was terminated at day 36. Significant inhibition of tumor growth was observed in the mice injected with the MSLN targeting trispecific antigen binding protein (containing the MH6T domain), compared to those injected with the vehicle control, as shown in FIG. 11.

### Example 12: Pharmacokinetics of MSLN Targeting Trispecific Antigen Binding Protein (Containing the MH6T Domain) in Cynomolgus Monkeys

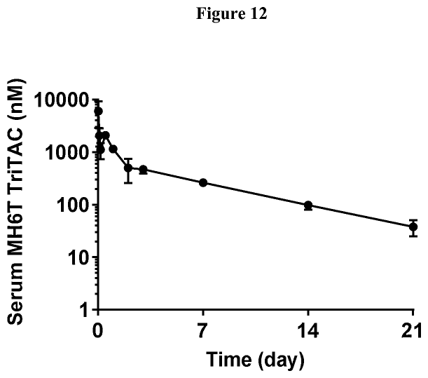

For this study, two cynomolgus monkeys were injected with 10 mg/kg dose of an MSLN targeting trispecific antigen binding protein (containing the MH6T domain; SEQ ID NO: 58), intravenously, and serum samples were collected at various time points after the injection. The amount of the MSLN targeting trispecific antigen binding protein (containing the MH6T domain) in the serum was measured using anti-idiotype antibodies recognizing the MSLN targeting trispecific antigen binding protein (containing the MH6T domain), in an electrochemiluminescent assay. FIG. 12 shows a plot for the serum MSLN targeting trispecific antigen binding protein (containing the MH6T domain) levels at various time points. The data was then used to calculate the pharmacokinetic properties of the MSLN targeting trispecific antigen binding protein (containing the MH6T domain), as provided in Table VII.

TABLE VII
Pharmacokinetic parameters for MSLN targeting trispecific
antigen binding protein (containing the MH6T domain)

| | | Cₘₐₓ | AUC, 0-inf | Clearance | Vss |
|---|---|---|---|---|---|
| Dose Level | Terminal t_{1/2} | (nM) | (hr*nM) | (mL/hr/kg) | (mL/kg) |
| 10 mg/kg | 112 | 6,130 | 355,000 | 0.58 | 70.0 |

Sequence Table

| | | |
|---|---|---|
| SEQ ID | 9B1 | QVQLVESGGGLVQPGGSLRLSCAASGRTFSVRGMAWYRQAGNNRALVATMNPDG |
| NO: 1 | | FPNYADAVKGRFTISWDIAENTVYLQMNSLNSEDTTVYYCNSGPYWGQGTQVTV |
| | | SS |
| SEQ ID | 9F3 | QVQLVESGGGLVQAGGSLRLSCAASGSIPSIEQMGWYRQAPGKQRELVAALTSG |
| NO: 2 | | GRANYADSVKGRFTISGDNVRNMVYLQMNSLKPEDTAIYYCSAGRFKGDYAQRS |
| | | GMDYWGKGTLVTVSS |
| SEQ ID | 7H2 | QVQLVESGGGLVQAGGSLRLSCAFSGTTYTFDLMSWYRQAPGKQRTVVASISSD |
| NO: 3 | | GRTSYADSVRGRFTISGENGKNTVYLQMNSLKLEDTAVYYCLGQRSGVRAFWGQ |
| | | GTQVTVSS |
| SEQ ID | 3B4 | QVQLVESGGGLVQAGGSLRLSCVASGSTSNINNMRWYRQAPGKERELVAVITRG |
| NO: 4 | | GYAIYLDAVKGRFTISRDNANNAIYLEMNSLKPEDTAVYVCNADRVEGTSGGPQ |
| | | LRDYFGQGTQVTVSS |
| SEQ ID | 4A2 | QVQLVESGGGLVQAGGSLRLSCAASGSTFGINAMGWYRQAPGKQRELVAVISRG |
| NO: 5 | | GSTNYADSVKGRFTISRDNAENTVSLQMNTLKPEDTAVYFCNARTYTRHDYWGQ |
| | | GTQVTVSS |
| SEQ ID | 12D1 | QVRLVESGGGLVQAGGSLRLSCAASISAFRLMSVRWYRQDPSKQREWVATIDQL |
| NO: 6 | | GRTNYADSVKGRFAISKDSTRNTVYLQMNMLRPEDTAVYYCNAGGGPLGSRWLR |
| | | GRHWGQGTQVTVSS |
| SEQ ID | 3G1 | QVRLVESGGGLVQAGESLRLSCAASGRPFSINTMGWYRQAPGKQRELVASISSS |
| NO: 7 | | GDFTYTDSVKGRFTISRDNAKNTVYLQMNSLKPEDTAVYYCNARRTYLPRRFGS |
| | | WGQGTQVTVSS |
| SEQ ID | 2A1 | QVQPVESGGGLVQPGGSLRLSCVVSGSDFTEDAMAWYRQASGKERESVAFVSKD |
| NO: 8 | | GKRILYLDSVRGRFTISRDIDKKTVYLQMDNLKPEDTGVYYCNSAPGAARNYWG |
| | | QGTQVTVSS |
| SEQ ID | 6F3 | QVQPVESGGGLVQPGGSLRLSCVVSGSDFTEDAMAWYRQASGKERESVAFVSKD |
| NO: 9 | | GKRILYLDSVRGRFTISRDIYKKTVYLQMDNLKPEDTGVYYCNSAPGAARNVWG |
| | | QGTQVTVSS |
| SEQ ID | 1H2 | EVQLVESGGGLVQPGNSLRLSCAASGFTFSSFGMSWVRQAPGKGLEWVSSISGS |
| NO: 10 | | GSDTLYADSVKGRFTISRDNAKTTLYLQMNSLRPEDTAVYYCTIGGSLSRSSQG |
| | | TLVTVSS |
| SEQ ID | 3F2 | QVQIVESGGGLVQAGGSLRLSCVASGLTYSIVAVGWYRQAPGKEREMVADISPV |
| NO: 11 | | GNTNYADSVKGRFTISKENAKNTVYLQMNSLKPEDTAVYYCHIVRGWLDERPGP |
| | | GPIVYWGQGTQVTVSS |
| SEQ ID | 12C2 | QVQLVESGGGLVQTGGSLRLSCAASGLTFGVYGMEWFRQAPGKQREWVASHTST |
| NO: 12 | | GYVYYRDSVKGRFTISRDNAKSTVYLQMNSLKPEDTAIYYCKANRGSYEYWGQG |
| | | TQVTVSS |
| SEQ ID | 2D1 | QVQLVESGGGLVQAGGSLRLSCAASTTSSINSMSWYRQAQGKQREPVAVITDRG |
| NO: 13 | | STSYADSVKGRFTISRDNAKNTVYLQMNSLKPEDTAIYTCHVIADWRGYWGQGT |
| | | QVTVSS |
| SEQ ID | 6H2 | QVQLVESGGGLVQAGGSLRLSCAASGRTLSRYAMGWFRQAPGKERQFVAAISRS |
| NO: 14 | | GGTTRYSDSVKGRFTISRDNAANTFYLQMNNLRPDDTAVYYCNVRRRGWGRTLE |
| | | YWGQGTQVTVSS |
| SEQ ID | 5D2 | QVQLGESGGGLVQAGGSLRLSCAASGSIFSPNAMIWHRQAPGKQREPVASINSS |
| NO: 15 | | GSTNYGDSVKGRFTVSRDIVKNTMYLQMNSLKPEDTAVYYCSYSDFRRGTQYWG |
| | | QGTQVTVSS |
| SEQ ID | 7C4 | QVQLVESGGGLVPSGGSLRLSCAASGATSAITNLGWYRRAPGQVREMVARISVR |
| NO: 16 | | EDKEDYEDSVKGRFTISRDNTQNLVYLQMNNLQPHDTAIYYCGAQRWGRGPGTT |
| | | WGQGTQVTVSS |
| SEQ ID | 5F2 | QVQLVESGGGLVQAGGSLRLSCAASGSTFRIRVMRWYRQAPGTERDLVAVISGS |
| NO: 17 | | STYYADSVKGRFTISRDNAKNTLYLQMNNLKPEDTAVYYCNADDSGIARDYWGQ |
| | | GTQVTVSS |
| SEQ ID | 2C2 | QVQLVESGGGLVQAGESRRLSCAVSGDTSKFKAVGWYRQAPGAQRELLAWINNS |
| NO: 18 | | GVGNTAESVKGRFTISRDNAKNTVYLQMNRLTPEDTDVYYCRFYRRFGINKNYW |
| | | GQGTQVTVSS |
| SEQ ID | 5G2 | QVQLVESGGGLVQAGGSLRLSCAASGSTFGNKPMGWYRQAPGKQRELVAVISSD |
| NO: 19 | | GGSTRYAALVKGRFTISRDNAKNTVYLQMESLVAEDTAVYYCNALRTYYLNDPV |
| | | VFSWGQGTQVTVSS |
| SEQ ID | 9H2 | QVQLVESGGGLVQAGGSLRLSCAASGSTSSINTMYWYRQAPGKERELVAFISSG |
| NO: 20 | | GSTNVRDSVKGRFSVSRDSAKNIVYLQMNSLTPEDTAVYYCNTYIPLRGTLHDY |
| | | WGQGTQVTVSS |
| SEQ ID | 5D4 | QVQLVESGGGLVQAGGSLRLSCVASGRTDRITTMGWYRQAPGKQRELVATISNR |
| NO: 21 | | GTSNYANSVKGRFTISRDNAKNTVYLQMNSLKPEDTAVYYCNARKWGRNYWGQG |
| | | TQVTVSS |
| SEQ ID | 2A4 | QVQLVESGGGLVQARGSLRLSCTASGRTIGINDMAWYRQAPGNQRELVATITKG |
| NO: 22 | | GTTDYADSVDGRFTISRDNAKNTVYLQMNSLKPEDTAVYYCNTKRREWAKDFEY |
| | | WGQGTQVTVSS |
| SEQ ID | 7F1 | QVQLVESGGGLVQAGGSLRLSCAASAIGSINSMSWYRQAPGKQREPVAVITDRG |
| NO: 23 | | STSYADSVKGRFTISRDNAKNTVYLQMNSLKPEDTAIYTCHVIADWRGYWGQGT |
| | | QVTVSS |
| SEQ ID | 5C2 | QVQLVESGGGLVQAGGSLRLSCAASGSTSSINTMYWFRQAPGEERELVATINRG |
| NO: 24 | | GSTNVRDSVKGRFSVSRDSAKNIVYLQMNRLKPEDTAVYYCNTYIPYGGTLHDF |
| | | WGQGTQVTVSS |
| SEQ ID | 2F4 | QVQLVESGGGLVQAGGSLRLSCTTSTTFSINSMSWYRQAPGNQREPVAVITNRG |
| NO: 25 | | TTSYADSVKGRFTISRDNARNTVYLQMDSLKPEDTAIYTCHVIADWRGYWGQGT |
| | | QVTVSS |
| SEQ ID | 2A2 | QVQLVESGGGLVQAGGSLTLSCAASGSTFSIRAMRWYRQAPGTERDLVAVIYGS |
| NO: 26 | | STYYADAVKGRFTISRDNAKNTLYLQMNNLKPEDTAVYYCNADTIGTARDYWGQ |
| | | GTQVTVSS |
| SEQ ID | 11F3 | QVQLVESGGGLVQAGGSLRLSCVASGRTSTIDTMYWHRQAPGNERELVAYVTSR |
| NO: 27 | | GTSNVADSVKGRFTISRDNAKNTAYLQMNSLKPEDTAVYYCSVRTTSYPVDFWG |
| | | QGTQVTVSS |
| SEQ ID | 10B3 | QVQLVESGGGLVQAGGSLRLSCAASGSTSSINTMYWYRQAPGKERELVAFISSG |
| NO: 28 | | GSTNVRDSVKGRFSVSRDSAKNIVYLQMNSLKPEDTAVYYCNTYIPYGGTLHDF |
| | | WGQGTQVTVSS |
| SEQ ID | 5H1 | QVQLVESGGGLVQPGGSLRLSCAASGGDWSANFMYWYRQAPGKQRELVARISGR |
| NO: 29 | | GVVDYVESVKGRFTISRDNAKNTVYLQMNSLKPEDTAVYYCAVASYWGQGTQVT |
| | | VSS |
| SEQ ID | MH1(humanized | EVQLVESGGGLVQPGGSLRLSCAASGGDWSANFMYWYRQAPGKQRELVARISGR |
| NO: 30 | version of | GVVDYVESVKGRFTISRDNSKNTLYLQMNSLRAEDTAVYYCAVASYWGQGTLVT |
| | 5H1) | VSS |
| SEQ ID | MH2 | EVQLVESGGGLVQPGGSLRLSCAASGGDWSANFMYWVRQAPGKGLEWVSRISGR |
| NO: 31 | (humanized | GVVDYVESVKGRFTISRDNSKNTLYLQMNSLRAEDTAVYYCAVASYWGQGTLVT |
| | version of | VSS |
| | 5H1) | |
| SEQ ID | MH3 | EVQLVESGGGLVQAGGSLRLSCAASGSTSSINTMYWYRQAPGKERELVAFISSG |
| NO: 32 | (humanized | GSTNVRDSVKGRFTISRDNSKNTLYLQMNSLRAEDTAVYYCNTYIPYGGTLHDF |
| | version of | WGQGTLVTVSS |
| | 10B3) | |
| SEQ ID | MH4 | EVQLVESGGGLVQPGGSLRLSCAASGSTSSINTMYWYRQAPGKERELVAFISSG |
| NO: 33 | (humanized | GSTNVRDSVKGRFTISRDNSKNTLYLQMNSLRAEDTAVYYCNTYIPYGGTLHDF |
| | version of | WGQGTLVTVSS |
| | 10B3) | |
| SEQ ID | MH5 | EVQLVESGGGLVQPGGSLRLSCAASGSTSSINTMYWVRQAPGKGLEWVSFISSG |
| NO: 34 | (humanized | GSTNVRDSVKGRFTISRDNSKNTLYLQMNSLRAEDTAVYYCNTYIPYGGTLHDF |
| | version of | WGQGTLVTVSS |
| | 10B3) | |
| SEQ ID | MH6-GG | QVQLVESGGGVVQAGGSLRLSCAASGSTFSIRAMRWYRQAPGTERDLVAVIYGS |
| NO: 35 | (humanized | STYYADAVKGRFTISRDNSKNTLYLQMNSLRAEDTAVYYCNADTIGTARDYWGQ |
| | version of | GTLVTVSS |
| | 2A2) | |
| SEQ ID | MH7-GG | QVQLVESGGGVVQPGGSLRLSCAASGSTFSIRAMRWYRQAPGKERELVAVIYGS |
| NO: 36 | (humanized | STYYADAVKGRFTISRDNSKNTLYLQMNSLRAEDTAVYYCNADTIGTARDYWGQ |
| | version of | GTLVTVSSGG |
| | 2A2) | |
| SEQ ID | MH8-GG | QVQLVESGGGVVQPGGSLRLSCAASGSTFSIRAMRWVRQAPGKGLEWVSVIYGS |
| NO: 37 | (humanized | STYYADAVKGRFTISRDNSKNTLYLQMNSLRAEDTAVYYCNADTIGTARDYWGQ |
| | version of | GTLVTVSSGG |
| | 2A2) | |
| SEQ ID | MH9 | EVQLVESGGGLVQAGGSLRLSCVASGRTSTIDTMYWHRQAPGNERELVAYVTSR |
| NO: 38 | (humanized | GTSNVADSVKGRFTISRDNSKNTLYLQMNSLRAEDTAVYYCSVRTTSYPVDFWG |
| | version of | QGTLVTVSGG |
| | 11F3) | |
| SEQ ID | MH10 | EVQLVESGGGLVQPGGSLRLSCAASGRTSTIDTMYWHRQAPGKERELVAYVTSR |
| NO: 39 | (humanized | GTSNVADSVKGRFTISRDNSKNTLYLQMNSLRAEDTAVYYCSVRTTSYPVDFWG |
| | version of | QGTLVTVSS |
| | 11F3) | |
| SEQ ID | MH11 | EVQLVESGGGLVQPGGSLRLSCAASGRTSTIDTMYWVRQAPGKGLEWVSYVTSR |
| NO: 40 | (humanized | GTSNVADSVKGRFTISRDNSKNTLYLQMNSLRAEDTAVYYCSVRTTSYPVDFWG |
| | version of | QGTLVTVSS |
| | 11F3) | |
| SEQ ID | Exemplary | ESGGGLV |
| NO: 41 | conserved | |
| | region in | |
| | MSLN binding | |
| | domain | |
| SEQ ID | Exemplary | LSC |
| NO: 42 | conserved | |
| | region in | |
| | MSLN binding | |
| | domain | |
| SEQ ID | Exemplary | GRF |
| NO: 43 | conserved | |
| | region in | |
| | MSLN binding | |
| | domain | |
| SEQ ID | Exemplary | VTVSS |
| NO: 44 | conserved | |
| | region in | |
| | MSLN binding | |
| | domain | |
| SEQ ID | Exemplary | QLVESGGG |
| NO: 45 | conserved | |
| | region in | |
| | MSLN binding | |
| | domain | |
| SEQ ID | Exemplary | GGSLRLSCAASG |
| NO: 46 | conserved | |
| | region in | |
| | MSLN binding | |
| | domain | |
| SEQ ID | Exemplary | ASG |
| NO: 47 | conserved | |
| | region in | |
| | MSLN binding | |
| | domain | |
| SEQ ID | Exemplary | RQAPG |
| NO: 48 | conserved | |
| | region in | |
| | MSLN binding | |
| | domain | |
| SEQ ID | Exemplary | VKGRFTISRDNSKNTLYLQMNSLRAEDTAVYYC |
| NO: 49 | conserved | |
| | region in | |
| | MSLN binding | |
| | domain | |
| SEQ ID | Exemplary | WGQGTLVTVSS |
| NO: 50 | conserved | |
| | region in | |
| | MSLN binding | |
| | domain | |
| SEQ ID | Exemplary | GRTFSVRGMA |
| NO: 51 | CDR1 of MSLN | |
| | binding | |
| | domain | |
| SEQ ID | Exemplary | INSSGSTNYG |
| NO: 52 | CDR2 of MSLN | |
| | binding | |
| | domain | |
| SEQ ID | Exemplary | NAGGGPLGSR |
| NO: 53 | CDR3 of MSLN | |
| | binding | |
| | domain | |
| SEQ ID | Exemplary | GGDWSANFMY |
| NO: 54 | CDR1 of MSLN | |
| | binding | |
| | domain | |
| SEQ ID | Exemplary | ISSGGSTNVR |
| NO: 55 | CDR2 of MSLN | |
| | binding | |
| | domain | |
| SEQ ID | Exemplary | NADTIGTARD |
| NO: 56 | CDR3 of MSLN | |
| | binding | |
| | domain | |
| SEQ ID | Mesothelin | MALPTARPLLGSCGTPALGSLLFLLFSLGWVQPSRTLAGETGQEAAPLDGVLAN |
| NO: 57 | protein | PPNISSLSPRQLLGFPCAEVSGLSTERVRELAVALAQKNVKLSTEQLRCLAHRL |
| | sequence | SEPPEDLDALPLDLLLFLNPDAFSGPQACTRFFSRITKANVDLLPRGAPERQRL |
| | | LPAALACWGVRGSLLSEADVRALGGLACDLPGRFVAESAEVLLPRLVSCPGPLD |
| | | QDQQEAARAALQGGGPPYGPPSTWSVSTMDALRGLLPVLGQPIIRSIPQGIVAA |
| | | WRQRSSRDPSWRQPERTILRPRFRREVEKTACPSGKKAREIDESLIFYKKWELE |
| | | ACVDAALLATQMDRVNAIPFTYEQLDVLKHKLDELYPQGYPESVIQHLGYLFLK |
| | | MSPEDIRKWNVTSLETLKALLEVNKGHEMSPQAPRRPLPQVATLIDRFVKGRGQ |
| | | LDKDTLDTLTAFYPGYLCSLSPEELSSVPPSSIWAVRPQDLDTCDPRQLDVLYP |
| | | KARLAFQNMNGSEYFVKIQSFLGGAPTEDLKALSQQNVSMDLATFMKLRTDAVL |
| | | PLTVAEVQKLLGPHVEGLKAEERHRPVRDWILRQRQDDLDTLGLGLQGGIPNGY |
| | | LVLDLSMQEALSGTPCLLGPGPVLTVLALLLASTLA |
| SEQ ID | MH6T | QVQLVESGGGVVQAGGSLTLSCAASGSTFSIRAMRWYRQAPGTERDLVAVIYGS |
| NO: 58 | (exemplary | STYYADAVKGRFTISRDNSKNTLYLQMNSLRAEDTAVYYCNADTIGTARDYWGQ |
| | MSLN binding | GTLVTVSS |
| | domain) | |
| SEQ ID | A trispecific | QVQLVESGGALVQPGGSLRLSCAASGFPVNRYSMRWYRQAPGKEREWVAGMSSA |
| NO: 59 | molecule | GDRSSYEDSVKGRFTISRDDARNTVYLQMNSLKPEDTAVYYCNVNVGFEYWGQG |
| | containing a | TQVTVSSGGGGSGGGSEVQLVESGGGLVQPGNSLRLSCAASGFTFSKFGMSWVR |
| | GFP binding | QAPGKGLEWVSSISGSGRDTLYADSVKGRFTISRDNAKTTLYLQMNSLRPEDTA |
| | domain | VYYCTIGGSLSVSSQGTLVTVSSGGGGSGGGSEVQLVESGGGLVQPGGSLKLSC |
| | | AASGFTFNKYAINWVRQAPGKGLEWVARIRSKYNNYATYYADQVKDRFTISRDD |
| | | SKNTAYLQMNNLKTEDTAVYYCVRHANFGNSYISYWAYWGQGTLVTVSSGGGGS |
| | | GGGGSGGGGSQTVVTQEPSLTVSPGGTVTLTCASSTGAVTSGNYPNWVQQKPGQ |
| | | APRGLIGGTKFLVPGTPARFSGSLLGGKAALTLSGVQPEDEAEYYCTLWYSNRW |
| | | VFGGGTKLTVLHHHHHH |
| SEQ ID | MH6 | QVQLVESGGGVVQAGGSLRLSCAASGSTFSIRAMRWYRQAPGTERDLVAVIYGS |
| NO: 60 | (exemplary | STYYADAVKGRFTISRDNSKNTLYLQMNSLRAEDTAVYYCNADTIGTARDYWGQ |
| | humanized | GTLVTVSS |
| | version of | |
| | 2A2) | |
| SEQ ID | MH7 | QVQLVESGGGVVQPGGSLRLSCAASGSTFSIRAMRWYRQAPGKERELVAVIYGS |
| NO: 61 | (exemplary | STYYADAVKGRFTISRDNSKNTLYLQMNSLRAEDTAVYYCNADTIGTARDYWGQ |
| | humanized | GTLVTVSS |
| | version of | |
| | 2A2) | |
| SEQ ID | MH8 | QVQLVESGGGVVQPGGSLRLSCAASGSTFSIRAMRWVRQAPGKGLEWVSVIYGS |
| NO: 62 | (humanized | STYYADAVKGRFTISRDNSKNTLYLQMNSLRAEDTAVYYCNADTIGTARDYWGQ |
| | version of | GTLVTVSS |
| | 2A2) | |

CDR1 Sequences for Various Exemplary MSLN Binding Domains of this Disclosure

| | Exemplary | |
|---|---|---|
| Sequence | MSLN binding | |
| ID No. | domain | CDR1 Sequence |
| 63 | 9B1 | GRTFSVRGMA |
| 64 | 9F3 | GSIPSIEQMG |
| 65 | 7H2 | GTTYTFDLMS |
| 66 | 3B4 | GSTSNINNMR |
| 67 | 4A2 | GSTFGINAMG |
| 68 | 12D1 | ISAFRLMSVR |
| 69 | 3G1 | GRPFSINTMG |
| 70 | 2A1 | GSDFTEDAMA |
| 71 | 6F3 | GSDFTEDAMA |
| 72 | 1H2 | GFTFSSFGMS |
| 73 | 3F2 | GLTYSIVAVG |
| 74 | 12C2 | GLTFGVYGME |
| 75 | 2D1 | TTSSINSMS |
| 76 | 6H2 | GRTLSRYAMG |
| 77 | 5D2 | GSIFSPNAMI |
| 78 | 7C4 | GATSAITNLG |
| 79 | 5F2 | GSTFRIRVMR |
| 80 | 2C2 | GDTSKFKAVG |
| 81 | 5G2 | GSTFGNKPMG |
| 82 | 9H2 | GSTSSINTMY |
| 83 | 5D4 | GRTDRITTMG |
| 84 | 2A4 | GRTIGINDMA |
| 85 | 7F1 | AIGSINSMS |
| 86 | 5C2 | GSTSSINTMY |
| 87 | 2F4 | TTFSINSMS |
| 88 | 2A2 | GSTFSIRAMR |
| 89 | 11F3 | GRTSTIDTMY |
| 90 | 10B3 | GSTSSINTMY |
| 91 | MH1 | GGDWSANFMY |
| 92 | MH2 | GGDWSANFMY |
| 93 | MH3 | GSTSSINTMY |
| 94 | MH4 | GSTSSINTMY |
| 95 | MH5 | GSTSSINTMY |
| 96 | MH6 | GSTFSIRAMR |
| 97 | MH7 | GSTFSIRAMR |
| 98 | MH8 | GSTFSIRAMR |
| 99 | MH9 | GRTSTIDTMY |
| 100 | MH10 | GRTSTIDTMY |
| 101 | MH11 | GRTSTIDTMY |

CDR2 Sequences for Various Exemplary MSLN Binding Domains of this Disclosure

| Sequence | Exemplary MSLN | |
|---|---|---|
| ID No. | binding domain | CDR2 Sequence |
| 102 | 9B1 | TMNPDGFPNYADAVKGRFT |
| 103 | 9F3 | ALTSGGRANYADSVKGRFT |
| 104 | 7H2 | SISSDGRTSYADSVRGRFT |
| 105 | 3B4 | VITRGGYAIYLDAVKGRFT |
| 106 | 4A2 | VISRGGSTNYADSVKGRFT |
| 107 | 12D1 | TIDQLGRTNYADSVKGRFA |
| 108 | 3G1 | SISSSGDFTYTDSVKGRFT |
| 109 | 2A1 | FVSKDGKRILYLDSVRGRFT |
| 110 | 6F3 | FVSKDGKRILYLDSVRGRFT |
| 111 | 1H2 | SISGSGSDTLYADSVKGRFT |
| 112 | 3F2 | DISPVGNTNYADSVKGRFT |
| 113 | 12C2 | SHTSTGYVYYRDSVKGRFT |
| 114 | 2D1 | VITDRGSTSYADSVKGRFT |
| 115 | 6H2 | AISRSGGTTRYSDSVKGRFT |
| 116 | 5D2 | SINSSGSTNYGDSVKGRFT |
| 117 | 7C4 | RISVREDKEDYEDSVKGRFT |
| 118 | 5F2 | VISGSSTYYADSVKGRFT |
| 119 | 2C2 | WINNSGVGNTAESVKGRFT |
| 120 | 5G2 | VISSDGGSTRYAALVKGRFT |
| 121 | 9H2 | FISSGGSTNVRDSVKGRFS |
| 122 | 5D4 | TISNRGTSNYANSVKGRFT |
| 123 | 2A4 | TITKGGTTDYADSVDGRFT |
| 124 | 7F1 | VITDRGSTSYADSVKGRFT |
| 125 | 5C2 | TINRGGSTNVRDSVKGRFS |
| 126 | 2F4 | VITNRGTTSYADSVKGRFT |
| 127 | 2A2 | VIYGSSTYYADAVKGRFT |
| 128 | 11F3 | YVTSRGTSNVADSVKGRFT |
| 129 | 10B3 | FISSGGSTNVRDSVKGRFS |
| 130 | MH1 | RISGRGVVDYVESVKGRFT |
| 131 | MH2 | RISGRGVVDYVESVKGRFT |
| 132 | MH3 | FISSGGSTNVRDSVKGRFT |
| 133 | MH4 | FISSGGSTNVRDSVKGRFT |
| 134 | MH5 | FISSGGSTNVRDSVKGRFT |
| 135 | MH6 | VIYGSSTYYADAVKGRFT |
| 136 | MH7 | VIYGSSTYYADAVKGRFT |
| 137 | MH8 | VIYGSSTYYADAVKGRFT |
| 138 | MH9 | YVTSRGTSNVADSVKGRFT |
| 139 | MH10 | YVTSRGTSNVADSVKGRFT |
| 140 | MH11 | YVTSRGTSNVADSVKGRFT |

CDR3 Sequences for Various Exemplary MSLN Binding Domains of this Disclosure

| Sequence | Exemplary MSLN | |
|---|---|---|
| ID No. | binding domain | CDR3 Sequence |
| 141 | 9B1 | GPY |
| 142 | 9F3 | GRFKGDYAQRSGMDY |
| 143 | 7H2 | QRSGVRAF |
| 144 | 3B4 | DRVEGTSGGPQLRDY |
| 145 | 4A2 | RTYTRHDY |
| 146 | 12D1 | GGGPLGSRWLRGRH |
| 147 | 3G1 | RRTYLPRRFGS |
| 148 | 2A1 | APGAARNY |
| 149 | 6F3 | APGAARNV |
| 150 | 1H2 | GGSLSRSS |
| 151 | 3F2 | VRGWLDERPGPGPIVY |
| 152 | 12C2 | NRGSYEY |
| 153 | 2D1 | IADWRGY |
| 154 | 6H2 | RRRGWGRTLEY |
| 155 | 5D2 | SDFRRGTQY |
| 156 | 7C4 | QRWGRGPGTT |
| 157 | 5F2 | DDSGIARDY |
| 158 | 2C2 | YRRFGINKNY |
| 159 | 5G2 | LRTYYLNDPVVFS |
| 160 | 9H2 | YIPLRGTLHDY |
| 161 | 5D4 | RKWGRNY |
| 162 | 2A4 | KRREWAKDFEY |
| 163 | 7F1 | IADWRGY |
| 164 | 5C2 | YIPYGGTLHDF |
| 165 | 2F4 | IADWRGY |
| 166 | 2A2 | DTIGTARDY |
| 167 | 11F3 | RTTSYPVDF |
| 168 | 10B3 | YIPYGGTLHDF |
| 169 | MH1 | ASY |
| 170 | MH2 | ASY |
| 171 | MH3 | YIPYGGTLHDF |
| 172 | MH4 | YIPYGGTLHDF |
| 173 | MH5 | YIPYGGTLHDF |
| 174 | MH6 | DTIGTARDY |
| 175 | MH7 | DTIGTARDY |
| 176 | MH8 | DTIGTARDY |
| 177 | MH9 | RTTSYPVDF |
| 178 | MH10 | RTTSYPVDF |
| 179 | MH11 | RTTSYPVDF |

Framework Region 1 (f1) Sequences for Various Exemplary MSLN Binding Domains

| | Exemplary | |
|---|---|---|
| | MSLN | |
| Sequence | binding | |
| ID No. | domain | Framework 1 |
| 180 | 9B1 | QVQLVESGGGLVQPGGSLRLSCAAS |
| 181 | 9F3 | QVQLVESGGGLVQAGGSLRLSCAAS |
| 182 | 7H2 | QVQLVESGGGLVQAGGSLRLSCAFS |
| 183 | 3B4 | QVQLVESGGGLVQAGGSLRLSCVAS |
| 184 | 4A2 | QVQLVESGGGLVQAGGSLRLSCAAS |
| 185 | 12D1 | QVRLVESGGGLVQAGGSLRLSCAAS |
| 186 | 3G1 | QVRLVESGGGLVQAGESLRLSCAAS |
| 187 | 2A1 | QVQPVESGGGLVQPGGSLRLSCVVS |
| 188 | 6F3 | QVQPVESGGGLVQPGGSLRLSCVVS |
| 189 | 1H2 | EVQLVESGGGLVQPGNSLRLSCAAS |
| 190 | 3F2 | QVQIVESGGGLVQAGGSLRLSCVAS |
| 191 | 12C2 | QVQLVESGGGLVQTGGSLRLSCAAS |
| 192 | 2D1 | QVQLVESGGGLVQAGGSLRLSCAAS |
| 193 | 6H2 | QVQLVESGGGLVQAGGSLRLSCAAS |
| 194 | 5D2 | QVQLGESGGGLVQAGGSLRLSCAAS |
| 195 | 7C4 | QVQLVESGGGLVPSGGSLRLSCAAS |
| 196 | 5F2 | QVQLVESGGGLVQAGGSLRLSCAAS |
| 197 | 2C2 | QVQLVESGGGLVQAGESRRLSCAVS |
| 198 | 5G2 | QVQLVESGGGLVQAGGSLRLSCAAS |
| 199 | 9H2 | QVQLVESGGGLVQAGGSLRLSCAAS |
| 200 | 5D4 | QVQLVESGGGLVQAGGSLRLSCVAS |
| 201 | 2A4 | QVQLVESGGGLVQARGSLRLSCTAS |
| 202 | 7F1 | QVQLVESGGGLVQAGGSLRLSCAAS |
| 203 | 5C2 | QVQLVESGGGLVQAGGSLRLSCAAS |
| 204 | 2F4 | QVQLVESGGGLVQAGGSLRLSCTTS |
| 205 | 2A2 | QVQLVESGGGLVQAGGSLTLSCAAS |
| 206 | 11F3 | QVQLVESGGGLVQAGGSLRLSCVAS |
| 207 | 10B3 | QVQLVESGGGLVQAGGSLRLSCAAS |
| 208 | MH1 | EVQLVESGGGLVQPGGSLRLSCAAS |
| 209 | MH2 | EVQLVESGGGLVQPGGSLRLSCAAS |
| 210 | MH3 | EVQLVESGGGLVQAGGSLRLSCAAS |
| 211 | MH4 | EVQLVESGGGLVQPGGSLRLSCAAS |
| 212 | MH5 | EVQLVESGGGLVQPGGSLRLSCAAS |
| 213 | MH6 | QVQLVESGGGVVQAGGSLRLSCAAS |
| 214 | MH7 | QVQLVESGGGVVQPGGSLRLSCAAS |
| 215 | MH8 | QVQLVESGGGVVQPGGSLRLSCAAS |
| 216 | MH9 | EVQLVESGGGLVQAGGSLRLSCVAS |
| 217 | MH10 | EVQLVESGGGLVQPGGSLRLSCAAS |
| 218 | MH11 | EVQLVESGGGLVQPGGSLRLSCAAS |

Framework Region 2 (f2) Sequences for Various Exemplary MSLN Binding Domains

| | Exemplary | |
|---|---|---|
| | MSLN | |
| Sequence | binding | |
| ID No. | domain | Framework 2 |
| 219 | 9B1 | WYRQAGNNRALVA |
| 220 | 9F3 | WYRQAPGKQRELVA |
| 221 | 7H2 | WYRQAPGKQRTVVA |
| 222 | 3B4 | WYRQAPGKERELVA |
| 223 | 4A2 | WYRQAPGKQRELVA |
| 224 | 12D1 | WYRQDPSKQREWVA |
| 225 | 3G1 | WYRQAPGKQRELVA |
| 226 | 2A1 | WYRQASGKERESVA |
| 227 | 6F3 | WYRQASGKERESVA |
| 228 | 1H2 | WVRQAPGKGLEWVS |
| 229 | 3F2 | WYRQAPGKEREMVA |
| 230 | 12C2 | WFRQAPGKQREWVA |
| 231 | 2D1 | WYRQAQGKQREPVA |
| 232 | 6H2 | WFRQAPGKERQFVA |
| 233 | 5D2 | WHRQAPGKQREPVA |
| 234 | 7C4 | WYRRAPGQVREMVA |
| 235 | 5F2 | WYRQAPGTERDLVA |
| 236 | 2C2 | WYRQAPGAQRELLA |
| 237 | 5G2 | WYRQAPGKQRELVA |
| 238 | 9H2 | WYRQAPGKERELVA |
| 239 | 5D4 | WYRQAPGKQRELVA |
| 240 | 2A4 | WYRQAPGNQRELVA |
| 241 | 7F1 | WYRQAPGKQREPVA |
| 242 | 5C2 | WFRQAPGEERELVA |
| 243 | 2F4 | WYRQAPGNQREPVA |
| 244 | 2A2 | WYRQAPGTERDLVA |
| 245 | 11F3 | WHRQAPGNERELVA |
| 246 | 10B3 | WYRQAPGKERELVA |
| 247 | MH1 | WYRQAPGKQRELVA |
| 248 | MH2 | WVRQAPGKGLEWVS |
| 249 | MH3 | WYRQAPGKERELVA |
| 250 | MH4 | WYRQAPGKERELVA |
| 251 | MH5 | WVRQAPGKGLEWVS |
| 252 | MH6 | WYRQAPGTERDLVA |
| 253 | MH7 | WYRQAPGKERELVA |
| 254 | MH8 | WVRQAPGKGLEWVS |
| 255 | MH9 | WHRQAPGNERELVA |
| 256 | MH10 | WHRQAPGKERELVA |
| 257 | MH11 | WVRQAPGKGLEWVS |

Framework Region 3 (f3) Sequences for Various Exemplary MSLN Binding Domains

| | Exemplary | |
|---|---|---|
| | MSLN | |
| Sequence | binding | |
| ID No. | domain | Framework 3 |
| 258 | 9B1 | ISWDIAENTVYLQMNSLNSEDTTVYYCNS |
| 259 | 9F3 | ISGDNVRNMVYLQMNSLKPEDTAIYYCSA |
| 260 | 7H2 | ISGENGKNTVYLQMNSLKLEDTAVYYCLG |
| 261 | 3B4 | ISRDNANNAIYLEMNSLKPEDTAVYVCNA |
| 262 | 4A2 | ISRDNAENTVSLQMNTLKPEDTAVYFCNA |
| 263 | 12D1 | ISKDSTRNTVYLQMNMLRPEDTAVYYCNA |
| 264 | 3G1 | ISRDNAKNTVYLQMNSLKPEDTAVYYCNA |
| 265 | 2A1 | ISRDIDKKTVYLQMDNLKPEDTGVYYCNS |
| 266 | 6F3 | ISRDIYKKTVYLQMDNLKPEDTGVYYCNS |
| 267 | 1H2 | ISRDNAKTTLYLQMNSLRPEDTAVYYCTI |
| 268 | 3F2 | ISKENAKNTVYLQMNSLKPEDTAVYYCHI |
| 269 | 12C2 | ISRDNAKSTVYLQMNSLKPEDTAIYYCKA |
| 270 | 2D1 | ISRDNAKNTVYLQMNSLKPEDTAIYTCHV |
| 271 | 6H2 | ISRDNAANTFYLQMNNLRPDDTAVYYCNV |
| 272 | 5D2 | VSRDIVKNTMYLQMNSLKPEDTAVYYCSY |
| 273 | 7C4 | ISRDNTQNLVYLQMNNLQPHDTAIYYCGA |
| 274 | 5F2 | ISRDNAKNTLYLQMNNLKPEDTAVYYCNA |
| 275 | 2C2 | ISRDNAKNTVYLQMNRLTPEDTDVYYCRF |
| 276 | 5G2 | ISRDNAKNTVYLQMESLVAEDTAVYYCNA |
| 277 | 9H2 | VSRDSAKNIVYLQMNSLTPEDTAVYYCNT |
| 278 | 5D4 | ISRDNAKNTVYLQMNSLKPEDTAVYYCNA |
| 279 | 2A4 | ISRDNAKNTVYLQMNSLKPEDTAVYYCNT |
| 280 | 7F1 | ISRDNAKNTVYLQMNSLKPEDTAIYTCHV |
| 281 | 5C2 | VSRDSAKNIVYLQMNRLKPEDTAVYYCNT |
| 282 | 2F4 | ISRDNARNTVYLQMDSLKPEDTAIYTCHV |
| 283 | 2A2 | ISRDNAKNTLYLQMNNLKPEDTAVYYCNA |
| 284 | 11F3 | ISRDNAKNTAYLQMNSLKPEDTAVYYCSV |
| 285 | 10B3 | VSRDSAKNIVYLQMNSLKPEDTAVYYCNT |
| 286 | MH1 | ISRDNSKNTLYLQMNSLRAEDTAVYYCAV |
| 287 | MH2 | ISRDNSKNTLYLQMNSLRAEDTAVYYCAV |
| 288 | MH3 | ISRDNSKNTLYLQMNSLRAEDTAVYYCNT |
| 289 | MH4 | ISRDNSKNTLYLQMNSLRAEDTAVYYCNT |
| 290 | MH5 | ISRDNSKNTLYLQMNSLRAEDTAVYYCNT |
| 291 | MH6 | ISRDNSKNTLYLQMNSLRAEDTAVYYCNA |
| 292 | MH7 | ISRDNSKNTLYLQMNSLRAEDTAVYYCNA |
| 293 | MH8 | ISRDNSKNTLYLQMNSLRAEDTAVYYCNA |
| 294 | MH9 | ISRDNSKNTLYLQMNSLRAEDTAVYYCSV |
| 295 | MH10 | ISRDNSKNTLYLQMNSLRAEDTAVYYCSV |
| 296 | MH11 | ISRDNSKNTLYLQMNSLRAEDTAVYYCSV |

Framework Region 4 (f4) Sequences for Various Exemplary MSLN Binding Domains

| | Exemplary | |
|---|---|---|
| | MSLN | |
| Sequence | binding | |
| ID No. | domain | Framework 4 |
| 297 | 9B1 | WGQGTQVTVSS |
| 298 | 9F3 | WGKGTLVTVSS |
| 299 | 7H2 | WGQGTQVTVSS |
| 300 | 3B4 | FGQGTQVTVSS |
| 301 | 4A2 | WGQGTQVTVSS |
| 302 | 12D1 | WGQGTQVTVSS |
| 303 | 3G1 | WGQGTQVTVSS |
| 304 | 2A1 | WGQGTQVTVSS |
| 305 | 6F3 | WGQGTQVTVSS |
| 306 | 1H2 | QGTLVTVSS |
| 307 | 3F2 | WGQGTQVTVSS |
| 308 | 12C2 | WGQGTQVTVSS |
| 309 | 2D1 | WGQGTQVTVSS |
| 310 | 6H2 | WGQGTQVTVSS |
| 311 | 5D2 | WGQGTQVTVSS |
| 312 | 7C4 | WGQGTQVTVSS |
| 313 | 5F2 | WGQGTQVTVSS |
| 314 | 2C2 | WGQGTQVTVSS |
| 315 | 5G2 | WGQGTQVTVSS |
| 316 | 9H2 | WGQGTQVTVSS |
| 317 | 5D4 | WGQGTQVTVSS |
| 318 | 2A4 | WGQGTQVTVSS |
| 319 | 7F1 | WGQGTQVTVSS |
| 320 | 5C2 | WGQGTQVTVSS |
| 321 | 2F4 | WGQGTQVTVSS |
| 322 | 2A2 | WGQGTQVTVSS |
| 323 | 11F3 | WGQGTQVTVSS |
| 324 | 10B3 | WGQGTQVTVSS |
| 325 | MH1 | WGQGTLVTVSS |
| 326 | MH2 | WGQGTLVTVSS |
| 327 | MH3 | WGQGTLVTVSS |
| 328 | MH4 | WGQGTLVTVSS |
| 329 | MH5 | WGQGTLVTVSS |
| 330 | MH6 | WGQGTLVTVSSGG |
| 331 | MH7 | WGQGTLVTVSSGG |
| 332 | MH8 | WGQGTLVTVSSGG |
| 333 | MH9 | WGQGTLVTVS |
| 334 | MH10 | WGQGTLVTVSS |
| 335 | MH11 | WGQGTLVTVSS |

1-30. (canceled)

31. A polynucleotide encoding a single domain mesothelin binding protein, wherein said protein comprises the following formula:
f1-r1-f2-r2-f3-r3-f4
wherein, r1 is CDR1; r2 is CDR2; and r3 is CDR3; and
wherein f1, f2, f3 and f4 are framework residues, and
wherein:
(i)
the CDR1 comprises the sequence GSTFSIRAMR (SEQ ID NO .: 88), the CDR2
comprises the sequence VIYGSSTYYADAVKGRFT (SEQ ID NO .: 127), and
the CDR3 comprises the sequence DTIGTARDY (SEQ ID No .: 166);
(ii)
the CDR1 comprises the sequence GRTSTIDTMY (SEQ ID NO .: 89), the CDR2
comprises YVTSRGTSNVADSVKGRFT (SEQ ID NO .: 128), and the CDR3
comprises the sequence RTTSYPVDF (SEQ ID NO .: 167);
iii)
the CDR1 comprises the sequence GSTSSINTMY (SEQ ID NO .: 82), the CDR2
comprises the sequence FISSGGSTNVRDSVKGRFT (SEQ ID NO. 132), the
CDR3 comprises the sequence YIPYGGTLHDF (SEQ ID NO .: 164);
(iv)
the CDR1 comprises the sequence GGDWSANFMY (SEQ ID NO .: 54), the
CDR2 comprises the sequence RISGRGVVDYVESVKGRFT (SEQ ID NO .:
130), the CDR3 comprises the sequence ASY (SEQ ID NO .: 169); or
(v)
the CDR1 comprises the sequence GSTFRIRVMR (SEQ ID NO: 79), the CDR2
comprises the sequence VISGSSTYYADSVKGRFT (SEQ ID NO: 118), and the
CDR3 comprises the sequence DDSGIARDY (SEQ ID NO: 157).

32. The polynucleotide of claim 31, wherein f1 comprises a sequence as set forth in any one of SEQ ID NOs.: 196, 205, and 208-218.

33. The polynucleotide of claim 31, wherein f2 comprises a sequence as set forth in any one of SEQ ID NOs.: 235, 244 and 247-257.

34. The polynucleotide of claim 31, wherein f3 comprises a sequence as set forth in any one of SEQ ID NOs.: 274, 283, and 286-296.

35. The polynucleotide of claim 31, wherein f4 comprises a sequence as set forth in any one of SEQ ID NOs.: 313, 322, and 325-335.

36. The polynucleotide of claim 31, wherein said protein comprises an amino acid sequence as set forth in any one of SEQ ID NOs.: 17, 26, 27, 32-40, 58 and 60-62.

37. A polynucleotide encoding a single domain mesothelin binding protein, wherein said protein comprises a sequence selected from the group consisting of SEQ ID NOs.: 1-16, 18-25, 28-29, and 31.

38. A polynucleotide encoding a single domain mesothelin binding protein, wherein said protein comprises the following formula:
f1-r1-f2-r2-f3-r3-f4
wherein, r1 is CDR1; r2 is CDR2; and r3 is CDR3; and
wherein f1, f2, f3 and f4 are framework residues, and
wherein:
(i)
the CDR1 comprises the sequence GSTSSINTMY (SEQ ID NO .: 82), the CDR2
comprises the sequence FISSGGSTNVRDSVKGRFS (SEQ ID NO .: 121), and
the CDR3 comprises the sequence YIPLRGTLHDY (SEQ ID NO .: 160);
(ii)
the CDR1 comprises the sequence GSTSSINTMY (SEQ ID NO .: 82), the CDR2
comprises TINRGGSTNVRDSVKGRFS (SEQ ID NO .: 125), and the CDR3
comprises the sequence YIPYGGTLHDF (SEQ ID NO .: 164);
(iii)
the CDR1 comprises the sequence TTFSINSMS (SEQ ID NO .: 87), the CDR2
comprises the sequence VITNRGTTSYADSVKGRFT (SEQ ID NO .: 126), the
CDR3 comprises the sequence IADWRGY (SEQ ID NO .: 153);
(iv)
the CDR1 comprises the sequence AIGSINSMS (SEQ ID NO .: 85), the CDR2
comprises the sequence VITDRGSTSYADSVKGRFT (SEQ ID NO .: 114), the
CDR3 comprises the sequence IADWRGY (SEQ ID NO .: 153);
(v)
the CDR1 comprises the sequence GLTFGVYGME (SEQ ID NO .: 74), the CDR2
comprises the sequence SHTSTGYVYYRDSVKGRFT (SEQ ID NO .: 113), and
the CDR3 comprises the sequence NRGSYEY (SEQ ID NO .: 152);
(vi)
the CDR1 comprises the sequence GSTSNINNMR (SEQ ID NO .: 66), the CDR2
comprises the sequence VITRGGYAIYLDAVKGRFT (SEQ ID NO .: 105), and
the CDR3 comprises the sequence DRVEGTSGGPQLRDY (SEQ ID NO .: 144);
or
(vii)
the CDR1 comprises the sequence GTTYTFDLMS (SEQ ID NO .: 65), the CDR2
comprises the sequence SISSDGRTSYADSVRGRFT (SEQ ID NO .: 104), and
the CDR3 comprises the sequence QRSGVRAF (SEQ ID NO .: 143).

39. The polynucleotide of claim 38, wherein f1 comprises a sequence as set forth in any one of SEQ ID NOs.: 180-218.

40. The polynucleotide of claim 38, wherein f2 comprises a sequence as set forth in any one of SEQ ID NOs.: 219-257.

41. The polynucleotide of claim 38, wherein f3 comprises a sequence as set forth in any one of SEQ ID NOs.: 258-296.

42. The polynucleotide of claim 38, wherein f4 comprises a sequence as set forth in any one of SEQ ID NOs.: 297-335.

43. The polynucleotide of claim 38, wherein said protein comprises a sequence selected from the group consisting of SEQ ID NOs.: 3, 4, 12, 20, 23, 24 and 25.

44. A vector comprising the polynucleotide of claim 31.

45. A host cell comprising the vector of claim 44.

46. A vector comprising the polynucleotide of claim 37.

47. A host cell comprising the vector of claim 46.

48. A vector comprising the polynucleotide of claim 38.

49. A host cell comprising the vector of claim 48.


SEQUENCE LISTING (336 sequences)

SEQ ID NO: 1 - protein, 110 aa, synthetic construct
  REGION 1..110: note = Description of Artificial Sequence: Synthetic polypeptide
QVQLVESGGGLVQPGGSLRLSCAASGRTFSVRGMAWYRQAGNNRALVATMNPDGFPNYADAVKGRFTISWDIAENTVYLQMNSLNSEDTTVYYCNSGPYWGQGTQVTVSS

SEQ ID NO: 2 - protein, 123 aa, synthetic construct
  REGION 1..123: note = Description of Artificial Sequence: Synthetic polypeptide
QVQLVESGGGLVQAGGSLRLSCAASGSIPSIEQMGWYRQAPGKQRELVAALTSGGRANYADSVKGRFTISGDNVRNMVYLQMNSLKPEDTAIYYCSAGRFKGDYAQRSGMDYWGKGTLVTVSS

SEQ ID NO: 3 - protein, 116 aa, synthetic construct
  REGION 1..116: note = Description of Artificial Sequence: Synthetic polypeptide
QVQLVESGGGLVQAGGSLRLSCAFSGTTYTFDLMSWYRQAPGKQRTVVASISSDGRTSYADSVRGRFTISGENGKNTVYLQMNSLKLEDTAVYYCLGQRSGVRAFWGQGTQVTVSS

SEQ ID NO: 4 - protein, 123 aa, synthetic construct
  REGION 1..123: note = Description of Artificial Sequence: Synthetic polypeptide
QVQLVESGGGLVQAGGSLRLSCVASGSTSNINNMRWYRQAPGKERELVAVITRGGYAIYLDAVKGRFTISRDNANNAIYLEMNSLKPEDTAVYVCNADRVEGTSGGPQLRDYFGQGTQVTVSS

SEQ ID NO: 5 - protein, 116 aa, synthetic construct
  REGION 1..116: note = Description of Artificial Sequence: Synthetic polypeptide
QVQLVESGGGLVQAGGSLRLSCAASGSTFGINAMGWYRQAPGKQRELVAVISRGGSTNYADSVKGRFTISRDNAENTVSLQMNTLKPEDTAVYFCNARTYTRHDYWGQGTQVTVSS

SEQ ID NO: 6 - protein, 122 aa, synthetic construct
  REGION 1..122: note = Description of Artificial Sequence: Synthetic polypeptide
QVRLVESGGGLVQAGGSLRLSCAASISAFRLMSVRWYRQDPSKQREWVATIDQLGRTNYADSVKGRFAISKDSTRNTVYLQMNMLRPEDTAVYYCNAGGGPLGSRWLRGRHWGQGTQVTVSS

SEQ ID NO: 7 - protein, 119 aa, synthetic construct
  REGION 1..119: note = Description of Artificial Sequence: Synthetic polypeptide
QVRLVESGGGLVQAGESLRLSCAASGRPFSINTMGWYRQAPGKQRELVASISSSGDFTYTDSVKGRFTISRDNAKNTVYLQMNSLKPEDTAVYYCNARRTYLPRRFGSWGQGTQVTVSS

SEQ ID NO: 8 - protein, 117 aa, synthetic construct
  REGION 1..117: note = Description of Artificial Sequence: Synthetic polypeptide
QVQPVESGGGLVQPGGSLRLSCVVSGSDFTEDAMAWYRQASGKERESVAFVSKDGKRILYLDSVRGRFTISRDIDKKTVYLQMDNLKPEDTGVYYCNSAPGAARNYWGQGTQVTVSS

SEQ ID NO: 9 - protein, 117 aa, synthetic construct
  REGION 1..117: note = Description of Artificial Sequence: Synthetic polypeptide
QVQPVESGGGLVQPGGSLRLSCVVSGSDFTEDAMAWYRQASGKERESVAFVSKDGKRILYLDSVRGRFTISRDIYKKTVYLQMDNLKPEDTGVYYCNSAPGAARNVWGQGTQVTVSS

SEQ ID NO: 10 - protein, 115 aa, synthetic construct [sequence omitted]

SEQ ID NO: 11 - protein, 124 aa, synthetic construct
  REGION 1..124: note = Description of Artificial Sequence: Synthetic polypeptide
QVQIVESGGGLVQAGGSLRLSCVASGLTYSIVAVGWYRQAPGKEREMVADISPVGNTNYADSVKGRFTISKENAKNTVYLQMNSLKPEDTAVYYCHIVRGWLDERPGPGPIVYWGQGTQVTVSS

SEQ ID NO: 12 - protein, 115 aa, synthetic construct
  REGION 1..115: note = Description of Artificial Sequence: Synthetic polypeptide
QVQLVESGGGLVQTGGSLRLSCAASGLTFGVYGMEWFRQAPGKQREWVASHTSTGYVYYRDSVKGRFTISRDNAKSTVYLQMNSLKPEDTAIYYCKANRGSYEYWGQGTQVTVSS

SEQ ID NO: 13 - protein, 114 aa, synthetic construct
  REGION 1..114: note = Description of Artificial Sequence: Synthetic polypeptide
QVQLVESGGGLVQAGGSLRLSCAASTTSSINSMSWYRQAQGKQREPVAVITDRGSTSYADSVKGRFTISRDNAKNTVYLQMNSLKPEDTAIYTCHVIADWRGYWGQGTQVTVSS

SEQ ID NO: 14 - protein, 120 aa, synthetic construct
  REGION 1..120: note = Description of Artificial Sequence: Synthetic polypeptide
QVQLVESGGGLVQAGGSLRLSCAASGRTLSRYAMGWFRQAPGKERQFVAAISRSGGTTRYSDSVKGRFTISRDNAANTFYLQMNNLRPDDTAVYYCNVRRRGWGRTLEYWGQGTQVTVSS

SEQ ID NO: 15 - protein, 117 aa, synthetic construct
  REGION 1..117: note = Description of Artificial Sequence: Synthetic polypeptide
QVQLGESGGGLVQAGGSLRLSCAASGSIFSPNAMIWHRQAPGKQREPVASINSSGSTNYGDSVKGRFTVSRDIVKNTMYLQMNSLKPEDTAVYYCSYSDFRRGTQYWGQGTQVTVSS

SEQ ID NO: 16 - protein, 119 aa, synthetic construct
  REGION 1..119: note = Description of Artificial Sequence: Synthetic polypeptide
QVQLVESGGGLVPSGGSLRLSCAASGATSAITNLGWYRRAPGQVREMVARISVREDKEDYEDSVKGRFTISRDNTQNLVYLQMNNLQPHDTAIYYCGAQRWGRGPGTTWGQGTQVTVSS

SEQ ID NO: 17 - protein, 116 aa, synthetic construct
  REGION 1..116: note = Description of Artificial Sequence: Synthetic polypeptide
QVQLVESGGGLVQAGGSLRLSCAASGSTFRIRVMRWYRQAPGTERDLVAVISGSSTYYADSVKGRFTISRDNAKNTLYLQMNNLKPEDTAVYYCNADDSGIARDYWGQGTQVTVSS

SEQ ID NO: 18 - protein, 118 aa, synthetic construct
  REGION 1..118: note = Description of Artificial Sequence: Synthetic polypeptide
QVQLVESGGGLVQAGESRRLSCAVSGDTSKFKAVGWYRQAPGAQRELLAWINNSGVGNTAESVKGRFTISRDNAKNTVYLQMNRLTPEDTDVYYCRFYRRFGINKNYWGQGTQVTVSS

SEQ ID NO: 19 - protein, 122 aa, synthetic construct
  REGION 1..122: note = Description of Artificial Sequence: Synthetic polypeptide
QVQLVESGGGLVQAGGSLRLSCAASGSTFGNKPMGWYRQAPGKQRELVAVISSDGGSTRYAALVKGRFTISRDNAKNTVYLQMESLVAEDTAVYYCNALRTYYLNDPVVFSWGQGTQVTVSS

SEQ ID NO: 20 - protein, 119 aa, synthetic construct
  REGION 1..119: note = Description of Artificial Sequence: Synthetic polypeptide
QVQLVESGGGLVQAGGSLRLSCAASGSTSSINTMYWYRQAPGKERELVAFISSGGSTNVRDSVKGRFSVSRDSAKNIVYLQMNSLTPEDTAVYYCNTYIPLRGTLHDYWGQGTQVTVSS

SEQ ID NO: 21 - protein, 115 aa, synthetic construct
  REGION 1..115: note = Description of Artificial Sequence: Synthetic polypeptide
QVQLVESGGGLVQAGGSLRLSCVASGRTDRITTMGWYRQAPGKQRELVATISNRGTSNYANSVKGRFTISRDNAKNTVYLQMNSLKPEDTAVYYCNARKWGRNYWGQGTQVTVSS

SEQ ID NO: 22 - protein, 119 aa, synthetic construct
  REGION 1..119: note = Description of Artificial Sequence: Synthetic polypeptide
QVQLVESGGGLVQARGSLRLSCTASGRTIGINDMAWYRQAPGNQRELVATITKGGTTDYADSVDGRFTISRDNAKNTVYLQMNSLKPEDTAVYYCNTKRREWAKDFEYWGQGTQVTVSS

SEQ ID NO: 23 - protein, 114 aa, synthetic construct
  REGION 1..114: note = Description of Artificial Sequence: Synthetic polypeptide
QVQLVESGGGLVQAGGSLRLSCAASAIGSINSMSWYRQAPGKQREPVAVITDRGSTSYADSVKGRFTISRDNAKNTVYLQMNSLKPEDTAIYTCHVIADWRGYWGQGTQVTVSS

SEQ ID NO: 24 - protein, 119 aa, synthetic construct
  REGION 1..119: note = Description of Artificial Sequence: Synthetic polypeptide
QVQLVESGGGLVQAGGSLRLSCAASGSTSSINTMYWFRQAPGEERELVATINRGGSTNVRDSVKGRFSVSRDSAKNIVYLQMNRLKPEDTAVYYCNTYIPYGGTLHDFWGQGTQVTVSS

SEQ ID NO: 25 - protein, 114 aa, synthetic construct
  REGION 1..114: note = Description of Artificial Sequence: Synthetic polypeptide
QVQLVESGGGLVQAGGSLRLSCTTSTTFSINSMSWYRQAPGNQREPVAVITNRGTTSYADSVKGRFTISRDNARNTVYLQMDSLKPEDTAIYTCHVIADWRGYWGQGTQVTVSS

SEQ ID NO: 26 - protein, 116 aa, synthetic construct
  REGION 1..116: note = Description of Artificial Sequence: Synthetic polypeptide
QVQLVESGGGLVQAGGSLTLSCAASGSTFSIRAMRWYRQAPGTERDLVAVIYGSSTYYADAVKGRFTISRDNAKNTLYLQMNNLKPEDTAVYYCNADTIGTARDYWGQGTQVTVSS

SEQ ID NO: 27 - protein, 117 aa, synthetic construct
  REGION 1..117: note = Description of Artificial Sequence: Synthetic polypeptide
QVQLVESGGGLVQAGGSLRLSCVASGRTSTIDTMYWHRQAPGNERELVAYVTSRGTSNVADSVKGRFTISRDNAKNTAYLQMNSLKPEDTAVYYCSVRTTSYPVDFWGQGTQVTVSS

SEQ ID NO: 28 - protein, 119 aa, synthetic construct
  REGION 1..119: note = Description of Artificial Sequence: Synthetic polypeptide
QVQLVESGGGLVQAGGSLRLSCAASGSTSSINTMYWYRQAPGKERELVAFISSGGSTNVRDSVKGRFSVSRDSAKNIVYLQMNSLKPEDTAVYYCNTYIPYGGTLHDFWGQGTQVTVSS

SEQ ID NO: 29 - protein, 111 aa, synthetic construct
  REGION 1..111: note = Description of Artificial Sequence: Synthetic polypeptide
QVQLVESGGGLVQPGGSLRLSCAASGGDWSANFMYWYRQAPGKQRELVARISGRGVVDYVESVKGRFTISRDNAKNTVYLQMNSLKPEDTAVYYCAVASYWGQGTQVTVSS

SEQ ID NO: 30 - protein, 111 aa, synthetic construct
  REGION 1..111: note = Description of Artificial Sequence: Synthetic polypeptide
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSS

SEQ ID NO: 31 - protein, 111 aa, synthetic construct
  REGION 1..111: note = Description of Artificial Sequence: Synthetic polypeptide
EVQLVESGGGLVQPGGSLRLSCAASGGDWSANFMYWVRQAPGKGLEWVSRISGRGVVDYVESVKGRFTISRDNSKNTLYLQMNSLRAEDTAVYYCAVASYWGQGTLVTVSS

SEQ ID NO: 32 - protein, 119 aa, synthetic construct
  REGION 1..119: note = Description of Artificial Sequence: Synthetic polypeptide
EVQLVESGGGLVQAGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 33 - protein, 119 aa, synthetic construct
  REGION 1..119: note = Description of Artificial Sequence: Synthetic polypeptide
EVQLVESGGGLVQPGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 34 - protein, 119 aa, synthetic construct
  REGION 1..119: note = Description of Artificial Sequence: Synthetic polypeptide
EVQLVESGGGLVQPGGSLRLSCAASGSTSSINTMYWVRQAPGKGLEWVSFISSGGSTNVRDSVKGRFTISRDNSKNTLYLQMNSLRAEDTAVYYCNTYIPYGGTLHDFWGQGTLVTVSS

SEQ ID NO: 35 - protein, 116 aa, synthetic construct
  REGION 1..116: note = Description of Artificial Sequence: Synthetic polypeptide
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 36 - protein, 118 aa, synthetic construct
  REGION 1..118: note = Description of Artificial Sequence: Synthetic polypeptide
QVQLVESGGGVVQPGGSLRLSCAASGSTFSIRAMRWYRQAPGKERELVAVIYGSSTYYADAVKGRFTISRDNSKNTLYLQMNSLRAEDTAVYYCNADTIGTARDYWGQGTLVTVSSGG

SEQ ID NO: 37 - protein, 118 aa, synthetic construct
  REGION 1..118: note = Description of Artificial Sequence: Synthetic polypeptide
QVQLVESGGGVVQPGGSLRLSCAASGSTFSIRAMRWVRQAPGKGLEWVSVIYGSSTYYADAVKGRFTISRDNSKNTLYLQMNSLRAEDTAVYYCNADTIGTARDYWGQGTLVTVSSGG

SEQ ID NO: 38 - protein, 118 aa, synthetic construct
  REGION 1..118: note = Description of Artificial Sequence: Synthetic polypeptide
EVQLVESGGGLVQAGGSLRLSCVASGRTSTIDTMYWHRQAPGNERELVAYVTSRGTSNVADSVKGRFTISRDNSKNTLYLQMNSLRAEDTAVYYCSVRTTSYPVDFWGQGTLVTVSGG

SEQ ID NO: 39 - protein, 117 aa, synthetic construct
  REGION 1..117: note = Description of Artificial Sequence: Synthetic polypeptide
EVQLVESGGGLVQPGGSLRLSCAASGRTSTIDTMYWHRQAPGKERELVAYVTSRGTSNVADSVKGRFTISRDNSKNTLYLQMNSLRAEDTAVYYCSVRTTSYPVDFWGQGTLVTVSS

SEQ ID NO: 40 - protein, 117 aa, synthetic construct
  REGION 1..117: note = Description of Artificial Sequence: Synthetic polypeptide
EVQLVESGGGLVQPGGSLRLSCAASGRTSTIDTMYWVRQAPGKGLEWVSYVTSRGTSNVADSVKGRFTISRDNSKNTLYLQMNSLRAEDTAVYYCSVRTTSYPVDFWGQGTLVTVSS

SEQ ID NO: 41 - protein, 7 aa, synthetic construct
  REGION 1..7: note = Description of Artificial Sequence: Synthetic peptide
ESGGGLV

SEQ ID NO: 42-43 - skipped (2 entries)

SEQ ID NO: 44 - protein, 5 aa, synthetic construct
  REGION 1..5: note = Description of Artificial Sequence: Synthetic peptide
VTVSS

SEQ ID NO: 45 - protein, 8 aa, synthetic construct
  REGION 1..8: note = Description of Artificial Sequence: Synthetic peptide
QLVESGGG

SEQ ID NO: 46 - protein, 12 aa, synthetic construct
  REGION 1..12: note = Description of Artificial Sequence: Synthetic peptide
GGSLRLSCAASG

SEQ ID NO: 47 - skipped

SEQ ID NO: 48 - protein, 5 aa, synthetic construct
  REGION 1..5: note = Description of Artificial Sequence: Synthetic peptide
RQAPG

SEQ ID NO: 49 - protein, 33 aa, synthetic construct
  REGION 1..33: note = Description of Artificial Sequence: Synthetic polypeptide
VKGRFTISRDNSKNTLYLQMNSLRAEDTAVYYC

SEQ ID NO: 50 - protein, 11 aa, synthetic construct
  REGION 1..11: note = Description of Artificial Sequence: Synthetic peptide
WGQGTLVTVSS

SEQ ID NO: 51 - protein, 10 aa, synthetic construct
  REGION 1..10: note = Description of Artificial Sequence: Synthetic peptide
GRTFSVRGMA

SEQ ID NO: 52 - protein, 10 aa, synthetic construct
  REGION 1..10: note = Description of Artificial Sequence: Synthetic peptide
INSSGSTNYG

SEQ ID NO: 53 - protein, 10 aa, synthetic construct
  REGION 1..10: note = Description of Artificial Sequence: Synthetic peptide
NAGGGPLGSR

SEQ ID NO: 54 - protein, 10 aa, synthetic construct
  REGION 1..10: note = Description of Artificial Sequence: Synthetic peptide
GGDWSANFMY

SEQ ID NO: 55 - protein, 10 aa, synthetic construct
  REGION 1..10: note = Description of Artificial Sequence: Synthetic peptide
ISSGGSTNVR

SEQ ID NO: 56 - protein, 10 aa, synthetic construct
  REGION 1..10: note = Description of Artificial Sequence: Synthetic peptide
NADTIGTARD

SEQ ID NO: 57 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 58 - protein, 116 aa, synthetic construct
  REGION 1..116: note = Description of Artificial Sequence: Synthetic polypeptide
QVQLVESGGGVVQAGGSLTLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 59 - protein, 503 aa, synthetic construct
  REGION 1..503: note = Description of Artificial Sequence: Synthetic polypeptide
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 60 - protein, 116 aa, synthetic construct; identical to SEQ ID NO: 35
  REGION 1..116: note = Description of Artificial Sequence: Synthetic polypeptide

SEQ ID NO: 61 - protein, 116 aa, synthetic construct
  REGION 1..116: note = Description of Artificial Sequence: Synthetic polypeptide
QVQLVESGGGVVQPGGSLRLSCAASGSTFSIRAMRWYRQAPGKERELVAVIYGSSTYYADAVKGRFTISRDNSKNTLYLQMNSLRAEDTAVYYCNADTIGTARDYWGQGTLVTVSS

SEQ ID NO: 62 - protein, 116 aa, synthetic construct
  REGION 1..116: note = Description of Artificial Sequence: Synthetic polypeptide
QVQLVESGGGVVQPGGSLRLSCAASGSTFSIRAMRWVRQAPGKGLEWVSVIYGSSTYYADAVKGRFTISRDNSKNTLYLQMNSLRAEDTAVYYCNADTIGTARDYWGQGTLVTVSS

SEQ ID NO: 63 - protein, 10 aa, synthetic construct
  REGION 1..10: note = Description of Artificial Sequence: Synthetic peptide
GRTFSVRGMA

SEQ ID NO: 64 - protein, 10 aa, synthetic construct
  REGION 1..10: note = Description of Artificial Sequence: Synthetic peptide
GSIPSIEQMG

SEQ ID NO: 65 - protein, 10 aa, synthetic construct
  REGION 1..10: note = Description of Artificial Sequence: Synthetic peptide
GTTYTFDLMS

SEQ ID NO: 66 - protein, 10 aa, synthetic construct
  REGION 1..10: note = Description of Artificial Sequence: Synthetic peptide
GSTSNINNMR

SEQ ID NO: 67 - protein, 10 aa, synthetic construct
  REGION 1..10: note = Description of Artificial Sequence: Synthetic peptide
GSTFGINAMG

SEQ ID NO: 68 - protein, 10 aa, synthetic construct
  REGION 1..10: note = Description of Artificial Sequence: Synthetic peptide
ISAFRLMSVR

SEQ ID NO: 69 - protein, 10 aa, synthetic construct
  REGION 1..10: note = Description of Artificial Sequence: Synthetic peptide
GRPFSINTMG

SEQ ID NO: 70 - protein, 10 aa, synthetic construct
  REGION 1..10: note = Description of Artificial Sequence: Synthetic peptide
GSDFTEDAMA

SEQ ID NO: 71 - protein, 10 aa, synthetic construct
  REGION 1..10: note = Description of Artificial Sequence: Synthetic peptide
GSDFTEDAMA

SEQ ID NO: 72 - protein, 10 aa, synthetic construct
  REGION 1..10: note = Description of Artificial Sequence: Synthetic peptide
GFTFSSFGMS

SEQ ID NO: 73 - protein, 10 aa, synthetic construct
  REGION 1..10: note = Description of Artificial Sequence: Synthetic peptide
GLTYSIVAVG

SEQ ID NO: 74 - protein, 10 aa, synthetic construct
  REGION 1..10: note = Description of Artificial Sequence: Synthetic peptide
GLTFGVYGME

SEQ ID NO: 75 - protein, 9 aa, synthetic construct
  REGION 1..9: note = Description of Artificial Sequence: Synthetic peptide
TTSSINSMS

SEQ ID NO: 76 - protein, 10 aa, synthetic construct
  REGION 1..10: note = Description of Artificial Sequence: Synthetic peptide
GRTLSRYAMG

SEQ ID NO: 77 - protein, 10 aa, synthetic construct
  REGION 1..10: note = Description of Artificial Sequence: Synthetic peptide
GSIFSPNAMI

SEQ ID NO: 78 - protein, 10 aa, synthetic construct
  REGION 1..10: note = Description of Artificial Sequence: Synthetic peptide
GATSAITNLG

SEQ ID NO: 79 - protein, 10 aa, synthetic construct
  REGION 1..10: note = Description of Artificial Sequence: Synthetic peptide
GSTFRIRVMR

SEQ ID NO: 80 - protein, 10 aa, synthetic construct
  REGION 1..10: note = Description of Artificial Sequence: Synthetic peptide
GDTSKFKAVG

SEQ ID NO: 81 - protein, 10 aa, synthetic construct
  REGION 1..10: note = Description of Artificial Sequence: Synthetic peptide
GSTFGNKPMG

SEQ ID NO: 82 - protein, 10 aa, synthetic construct
  REGION 1..10: note = Description of Artificial Sequence: Synthetic peptide
GSTSSINTMY

SEQ ID NO: 83 - protein, 10 aa, synthetic construct
  REGION 1..10: note = Description of Artificial Sequence: Synthetic peptide
GRTDRITTMG

SEQ ID NO: 84 - protein, 10 aa, synthetic construct
  REGION 1..10: note = Description of Artificial Sequence: Synthetic peptide
GRTIGINDMA

SEQ ID NO: 85 - protein, 9 aa, synthetic construct
  REGION 1..9: note = Description of Artificial Sequence: Synthetic peptide
AIGSINSMS

SEQ ID NO: 86 - protein, 10 aa, synthetic construct
  REGION 1..10: note = Description of Artificial Sequence: Synthetic peptide
GSTSSINTMY

SEQ ID NO: 87 - protein, 9 aa, synthetic construct
  REGION 1..9: note = Description of Artificial Sequence: Synthetic peptide
TTFSINSMS

SEQ ID NO: 88 - protein, 10 aa, synthetic construct
  REGION 1..10: note = Description of Artificial Sequence: Synthetic peptide
GSTFSIRAMR

SEQ ID NO: 89 - protein, 10 aa, synthetic construct
  REGION 1..10: note = Description of Artificial Sequence: Synthetic peptide
GRTSTIDTMY

SEQ ID NO: 90 - protein, 10 aa, synthetic construct
  REGION 1..10: note = Description of Artificial Sequence: Synthetic peptide
GSTSSINTMY

SEQ ID NO: 91 - protein, 10 aa, synthetic construct
  REGION 1..10: note = Description of Artificial Sequence: Synthetic peptide
GGDWSANFMY

SEQ ID NO: 92 - protein, 10 aa, synthetic construct
  REGION 1..10: note = Description of Artificial Sequence: Synthetic peptide
GGDWSANFMY

SEQ ID NO: 93 - protein, 10 aa, synthetic construct
  REGION 1..10: note = Description of Artificial Sequence: Synthetic peptide
GSTSSINTMY

SEQ ID NO: 94 - protein, 10 aa, synthetic construct
  REGION 1..10: note = Description of Artificial Sequence: Synthetic peptide
GSTSSINTMY

SEQ ID NO: 95 - protein, 10 aa, synthetic construct
  REGION 1..10: note = Description of Artificial Sequence: Synthetic peptide
GSTSSINTMY

SEQ ID NO: 96 - protein, 10 aa, synthetic construct
  REGION 1..10: note = Description of Artificial Sequence: Synthetic peptide
GSTFSIRAMR

SEQ ID NO: 97 - protein, 10 aa, synthetic construct
  REGION 1..10: note = Description of Artificial Sequence: Synthetic peptide
GSTFSIRAMR

SEQ ID NO: 98 - protein, 10 aa, synthetic construct
  REGION 1..10: note = Description of Artificial Sequence: Synthetic peptide
GSTFSIRAMR

SEQ ID NO: 99 - protein, 10 aa, synthetic construct
  REGION 1..10: note = Description of Artificial Sequence: Synthetic peptide
GRTSTIDTMY

SEQ ID NO: 100 - protein, 10 aa, synthetic construct
  REGION 1..10: note = Description of Artificial Sequence: Synthetic peptide
GRTSTIDTMY

SEQ ID NO: 101 - protein, 10 aa, synthetic construct
  REGION 1..10: note = Description of Artificial Sequence: Synthetic peptide
GRTSTIDTMY

SEQ ID NO: 102 - protein, 19 aa, synthetic construct
  REGION 1..19: note = Description of Artificial Sequence: Synthetic peptide
TMNPDGFPNYADAVKGRFT

SEQ ID NO: 103 - protein, 19 aa, synthetic construct
  REGION 1..19: note = Description of Artificial Sequence: Synthetic peptide
ALTSGGRANYADSVKGRFT

SEQ ID NO: 104 - protein, 19 aa, synthetic construct
  REGION 1..19: note = Description of Artificial Sequence: Synthetic peptide
SISSDGRTSYADSVRGRFT

SEQ ID NO: 105 - protein, 19 aa, synthetic construct
  REGION 1..19: note = Description of Artificial Sequence: Synthetic peptide
VITRGGYAIYLDAVKGRFT

SEQ ID NO: 106 - protein, 19 aa, synthetic construct
  REGION 1..19: note = Description of Artificial Sequence: Synthetic peptide
VISRGGSTNYADSVKGRFT

SEQ ID NO: 107 - protein, 19 aa, synthetic construct
  REGION 1..19: note = Description of Artificial Sequence: Synthetic peptide
TIDQLGRTNYADSVKGRFA

SEQ ID NO: 108 - protein, 19 aa, synthetic construct
  REGION 1..19: note = Description of Artificial Sequence: Synthetic peptide
SISSSGDFTYTDSVKGRFT

SEQ ID NO: 109 - protein, 20 aa, synthetic construct
  REGION 1..20: note = Description of Artificial Sequence: Synthetic peptide
FVSKDGKRILYLDSVRGRFT

SEQ ID NO: 110 - protein, 20 aa, synthetic construct
  REGION 1..20: note = Description of Artificial Sequence: Synthetic peptide
FVSKDGKRILYLDSVRGRFT

SEQ ID NO: 111 - protein, 20 aa, synthetic construct
  REGION 1..20: note = Description of Artificial Sequence: Synthetic peptide
SISGSGSDTLYADSVKGRFT

SEQ ID NO: 112 - protein, 19 aa, synthetic construct
  REGION 1..19: note = Description of Artificial Sequence: Synthetic peptide
DISPVGNTNYADSVKGRFT

SEQ ID NO: 113 - protein, 19 aa, synthetic construct
  REGION 1..19: note = Description of Artificial Sequence: Synthetic peptide
SHTSTGYVYYRDSVKGRFT

SEQ ID NO: 114 - protein, 19 aa, synthetic construct
  REGION 1..19: note = Description of Artificial Sequence: Synthetic peptide
VITDRGSTSYADSVKGRFT

SEQ ID NO: 115 - protein, 20 aa, synthetic construct
  REGION 1..20: note = Description of Artificial Sequence: Synthetic peptide
AISRSGGTTRYSDSVKGRFT

SEQ ID NO: 116 - protein, 19 aa, synthetic construct
  REGION 1..19: note = Description of Artificial Sequence: Synthetic peptide
SINSSGSTNYGDSVKGRFT

SEQ ID NO: 117 - protein, 20 aa, synthetic construct
  REGION 1..20: note = Description of Artificial Sequence: Synthetic peptide
RISVREDKEDYEDSVKGRFT

SEQ ID NO: 118 - protein, 18 aa, synthetic construct
  REGION 1..18: note = Description of Artificial Sequence: Synthetic peptide
VISGSSTYYADSVKGRFT

SEQ ID NO: 119 - protein, 19 aa, synthetic construct
  REGION 1..19: note = Description of Artificial Sequence: Synthetic peptide
WINNSGVGNTAESVKGRFT

SEQ ID NO: 120 - protein, 20 aa, synthetic construct
  REGION 1..20: note = Description of Artificial Sequence: Synthetic peptide
VISSDGGSTRYAALVKGRFT

SEQ ID NO: 121 - protein, 19 aa, synthetic construct
  REGION 1..19: note = Description of Artificial Sequence: Synthetic peptide
FISSGGSTNVRDSVKGRFS

SEQ ID NO: 122 - protein, 19 aa, synthetic construct
  REGION 1..19: note = Description of Artificial Sequence: Synthetic peptide
TISNRGTSNYANSVKGRFT

SEQ ID NO: 123 - protein, 19 aa, synthetic construct
  REGION 1..19: note = Description of Artificial Sequence: Synthetic peptide
TITKGGTTDYADSVDGRFT

SEQ ID NO: 124 - protein, 19 aa, synthetic construct
  REGION 1..19: note = Description of Artificial Sequence: Synthetic peptide
VITDRGSTSYADSVKGRFT

SEQ ID NO: 125 - protein, 19 aa, synthetic construct
  REGION 1..19: note = Description of Artificial Sequence: Synthetic peptide
TINRGGSTNVRDSVKGRFS

SEQ ID NO: 126 - protein, 19 aa, synthetic construct
  REGION 1..19: note = Description of Artificial Sequence: Synthetic peptide
VITNRGTTSYADSVKGRFT

SEQ ID NO: 127 - protein, 18 aa, synthetic construct
  REGION 1..18: note = Description of Artificial Sequence: Synthetic peptide
VIYGSSTYYADAVKGRFT

SEQ ID NO: 128 - protein, 19 aa, synthetic construct
  REGION 1..19: note = Description of Artificial Sequence: Synthetic peptide
YVTSRGTSNVADSVKGRFT

SEQ ID NO: 129 - protein, 19 aa, synthetic construct
  REGION 1..19: note = Description of Artificial Sequence: Synthetic peptide
FISSGGSTNVRDSVKGRFS

SEQ ID NO: 130 - protein, 19 aa, synthetic construct
  REGION 1..19: note = Description of Artificial Sequence: Synthetic peptide
RISGRGVVDYVESVKGRFT

SEQ ID NO: 131 - protein, 19 aa, synthetic construct
  REGION 1..19: note = Description of Artificial Sequence: Synthetic peptide
RISGRGVVDYVESVKGRFT

SEQ ID NO: 132 - protein, 19 aa, synthetic construct
  REGION 1..19: note = Description of Artificial Sequence: Synthetic peptide
FISSGGSTNVRDSVKGRFT

SEQ ID NO: 133 - protein, 19 aa, synthetic construct
  REGION 1..19: note = Description of Artificial Sequence: Synthetic peptide
FISSGGSTNVRDSVKGRFT

SEQ ID NO: 134 - protein, 19 aa, synthetic construct
  REGION 1..19: note = Description of Artificial Sequence: Synthetic peptide
FISSGGSTNVRDSVKGRFT

SEQ ID NO: 135 - protein, 18 aa, synthetic construct
  REGION 1..18: note = Description of Artificial Sequence: Synthetic peptide
VIYGSSTYYADAVKGRFT

SEQ ID NO: 136 - protein, 18 aa, synthetic construct
  REGION 1..18: note = Description of Artificial Sequence: Synthetic peptide
VIYGSSTYYADAVKGRFT

SEQ ID NO: 137 - protein, 18 aa, synthetic construct
  REGION 1..18: note = Description of Artificial Sequence: Synthetic peptide
VIYGSSTYYADAVKGRFT

SEQ ID NO: 138 - protein, 19 aa, synthetic construct
  REGION 1..19: note = Description of Artificial Sequence: Synthetic peptide
YVTSRGTSNVADSVKGRFT

SEQ ID NO: 139 - protein, 19 aa, synthetic construct
  REGION 1..19: note = Description of Artificial Sequence: Synthetic peptide
YVTSRGTSNVADSVKGRFT

SEQ ID NO: 140 - protein, 19 aa, synthetic construct
  REGION 1..19: note = Description of Artificial Sequence: Synthetic peptide
YVTSRGTSNVADSVKGRFT

SEQ ID NO: 141 - skipped

SEQ ID NO: 142 - protein, 15 aa, synthetic construct
  REGION 1..15: note = Description of Artificial Sequence: Synthetic peptide
GRFKGDYAQRSGMDY

SEQ ID NO: 143 - protein, 8 aa, synthetic construct
  REGION 1..8: note = Description of Artificial Sequence: Synthetic peptide
QRSGVRAF

SEQ ID NO: 144 - protein, 15 aa, synthetic construct
  REGION 1..15: note = Description of Artificial Sequence: Synthetic peptide
DRVEGTSGGPQLRDY

SEQ ID NO: 145 - protein, 8 aa, synthetic construct
  REGION 1..8: note = Description of Artificial Sequence: Synthetic peptide
RTYTRHDY

SEQ ID NO: 146 - protein, 14 aa, synthetic construct
  REGION 1..14: note = Description of Artificial Sequence: Synthetic peptide
GGGPLGSRWLRGRH

SEQ ID NO: 147 - protein, 11 aa, synthetic construct
  REGION 1..11: note = Description of Artificial Sequence: Synthetic peptide
RRTYLPRRFGS

SEQ ID NO: 148 - protein, 8 aa, synthetic construct
  REGION 1..8: note = Description of Artificial Sequence: Synthetic peptide
APGAARNY

SEQ ID NO: 149 - protein, 8 aa, synthetic construct
  REGION 1..8: note = Description of Artificial Sequence: Synthetic peptide
APGAARNV

SEQ ID NO: 150 - protein, 8 aa, synthetic construct
  REGION 1..8: note = Description of Artificial Sequence: Synthetic peptide
GGSLSRSS

SEQ ID NO: 151 - protein, 16 aa, synthetic construct
  REGION 1..16: note = Description of Artificial Sequence: Synthetic peptide
VRGWLDERPGPGPIVY

SEQ ID NO: 152 - protein, 7 aa, synthetic construct
  REGION 1..7: note = Description of Artificial Sequence: Synthetic peptide
NRGSYEY

SEQ ID NO: 153 - protein, 7 aa, synthetic construct
  REGION 1..7: note = Description of Artificial Sequence: Synthetic peptide
IADWRGY

SEQ ID NO: 154 - protein, 11 aa, synthetic construct
  REGION 1..11: note = Description of Artificial Sequence: Synthetic peptide
RRRGWGRTLEY

SEQ ID NO: 155 - protein, 9 aa, synthetic construct
  REGION 1..9: note = Description of Artificial Sequence: Synthetic peptide
SDFRRGTQY

SEQ ID NO: 156 - protein, 10 aa, synthetic construct
  REGION 1..10: note = Description of Artificial Sequence: Synthetic peptide
QRWGRGPGTT

SEQ ID NO: 157 - protein, 9 aa, synthetic construct
  REGION 1..9: note = Description of Artificial Sequence: Synthetic peptide
DDSGIARDY

SEQ ID NO: 158 - protein, 10 aa, synthetic construct
  REGION 1..10: note = Description of Artificial Sequence: Synthetic peptide
YRRFGINKNY

SEQ ID NO: 159 - protein, 13 aa, synthetic construct
  REGION 1..13: note = Description of Artificial Sequence: Synthetic peptide
LRTYYLNDPVVFS

SEQ ID NO: 160 - protein, 11 aa, synthetic construct
  REGION 1..11: note = Description of Artificial Sequence: Synthetic peptide
YIPLRGTLHDY

SEQ ID NO: 161 - protein, 7 aa, synthetic construct
  REGION 1..7: note = Description of Artificial Sequence: Synthetic peptide
RKWGRNY

SEQ ID NO: 162 - protein, 11 aa, synthetic construct
  REGION 1..11: note = Description of Artificial Sequence: Synthetic peptide
KRREWAKDFEY

SEQ ID NO: 163 - protein, 7 aa, synthetic construct
  REGION 1..7: note = Description of Artificial Sequence: Synthetic peptide
IADWRGY

SEQ ID NO: 164 - protein, 11 aa, synthetic construct
  REGION 1..11: note = Description of Artificial Sequence: Synthetic peptide
YIPYGGTLHDF

SEQ ID NO: 165 - protein, 7 aa, synthetic construct
  REGION 1..7: note = Description of Artificial Sequence: Synthetic peptide
IADWRGY

SEQ ID NO: 166 - protein, 9 aa, synthetic construct
  REGION 1..9: note = Description of Artificial Sequence: Synthetic peptide
DTIGTARDY

SEQ ID NO: 167 - protein, 9 aa, synthetic construct
  REGION 1..9: note = Description of Artificial Sequence: Synthetic peptide
RTTSYPVDF

SEQ ID NO: 168 - protein, 11 aa, synthetic construct
  REGION 1..11: note = Description of Artificial Sequence: Synthetic peptide
YIPYGGTLHDF

SEQ ID NO: 169-170 - skipped (2 entries)

SEQ ID NO: 171 - protein, 11 aa, synthetic construct
  REGION 1..11: note = Description of Artificial Sequence: Synthetic peptide
YIPYGGTLHDF

SEQ ID NO: 172 - protein, 11 aa, synthetic construct
  REGION 1..11: note = Description of Artificial Sequence: Synthetic peptide
YIPYGGTLHDF

SEQ ID NO: 173 - protein, 11 aa, synthetic construct
  REGION 1..11: note = Description of Artificial Sequence: Synthetic peptide
YIPYGGTLHDF

SEQ ID NO: 174 - protein, 9 aa, synthetic construct
  REGION 1..9: note = Description of Artificial Sequence: Synthetic peptide
DTIGTARDY

SEQ ID NO: 175 - protein, 9 aa, synthetic construct
  REGION 1..9: note = Description of Artificial Sequence: Synthetic peptide
DTIGTARDY

SEQ ID NO: 176 - protein, 9 aa, synthetic construct
  REGION 1..9: note = Description of Artificial Sequence: Synthetic peptide
DTIGTARDY

SEQ ID NO: 177 - protein, 9 aa, synthetic construct
  REGION 1..9: note = Description of Artificial Sequence: Synthetic peptide
RTTSYPVDF

SEQ ID NO: 178 - protein, 9 aa, synthetic construct
  REGION 1..9: note = Description of Artificial Sequence: Synthetic peptide
RTTSYPVDF

SEQ ID NO: 179 - protein, 9 aa, synthetic construct
  REGION 1..9: note = Description of Artificial Sequence: Synthetic peptide
RTTSYPVDF

SEQ ID NO: 180 - protein, 25 aa, synthetic construct
  REGION 1..25: note = Description of Artificial Sequence: Synthetic peptide
QVQLVESGGGLVQPGGSLRLSCAAS

SEQ ID NO: 181 - protein, 25 aa, synthetic construct
  REGION 1..25: note = Description of Artificial Sequence: Synthetic peptide
QVQLVESGGGLVQAGGSLRLSCAAS

SEQ ID NO: 182 - protein, 25 aa, synthetic construct
  REGION 1..25: note = Description of Artificial Sequence: Synthetic peptide
QVQLVESGGGLVQAGGSLRLSCAFS

SEQ ID NO: 183 - protein, 25 aa, synthetic construct
  REGION 1..25: note = Description of Artificial Sequence: Synthetic peptide
QVQLVESGGGLVQAGGSLRLSCVAS

SEQ ID NO: 184 - protein, 25 aa, synthetic construct
  REGION 1..25: note = Description of Artificial Sequence: Synthetic peptide
QVQLVESGGGLVQAGGSLRLSCAAS

SEQ ID NO: 185 - protein, 25 aa, synthetic construct
  REGION 1..25: note = Description of Artificial Sequence: Synthetic peptide
QVRLVESGGGLVQAGGSLRLSCAAS

SEQ ID NO: 186 - protein, 25 aa, synthetic construct
  REGION 1..25: note = Description of Artificial Sequence: Synthetic peptide
QVRLVESGGGLVQAGESLRLSCAAS

SEQ ID NO: 187 - protein, 25 aa, synthetic construct
  REGION 1..25: note = Description of Artificial Sequence: Synthetic peptide
QVQPVESGGGLVQPGGSLRLSCVVS

SEQ ID NO: 188 - protein, 25 aa, synthetic construct
  REGION 1..25: note = Description of Artificial Sequence: Synthetic peptide
QVQPVESGGGLVQPGGSLRLSCVVS

SEQ ID NO: 189 - protein, 25 aa, synthetic construct
  REGION 1..25: note = Description of Artificial Sequence: Synthetic peptide
EVQLVESGGGLVQPGNSLRLSCAAS

SEQ ID NO: 190 - protein, 25 aa, synthetic construct
  REGION 1..25: note = Description of Artificial Sequence: Synthetic peptide
QVQIVESGGGLVQAGGSLRLSCVAS

SEQ ID NO: 191 - protein, 25 aa, synthetic construct
  REGION 1..25: note = Description of Artificial Sequence: Synthetic peptide
QVQLVESGGGLVQTGGSLRLSCAAS

SEQ ID NO: 192 - protein, 25 aa, synthetic construct
  REGION 1..25: note = Description of Artificial Sequence: Synthetic peptide
QVQLVESGGGLVQAGGSLRLSCAAS

SEQ ID NO: 193 - protein, 25 aa, synthetic construct
  REGION 1..25: note = Description of Artificial Sequence: Synthetic peptide
QVQLVESGGGLVQAGGSLRLSCAAS

SEQ ID NO: 194 - protein, 25 aa, synthetic construct
  REGION 1..25: note = Description of Artificial Sequence: Synthetic peptide
QVQLGESGGGLVQAGGSLRLSCAAS

SEQ ID NO: 195 - protein, 25 aa, synthetic construct
  REGION 1..25: note = Description of Artificial Sequence: Synthetic peptide
QVQLVESGGGLVPSGGSLRLSCAAS

SEQ ID NO: 196 - protein, 25 aa, synthetic construct
  REGION 1..25: note = Description of Artificial Sequence: Synthetic peptide
QVQLVESGGGLVQAGGSLRLSCAAS

SEQ ID NO: 197 - protein, 25 aa, synthetic construct
  REGION 1..25: note = Description of Artificial Sequence: Synthetic peptide
QVQLVESGGGLVQAGESRRLSCAVS

SEQ ID NO: 198 - protein, 25 aa, synthetic construct
  REGION 1..25: note = Description of Artificial Sequence: Synthetic peptide
QVQLVESGGGLVQAGGSLRLSCAAS

SEQ ID NO: 199 - protein, 25 aa, synthetic construct
  REGION 1..25: note = Description of Artificial Sequence: Synthetic peptide
QVQLVESGGGLVQAGGSLRLSCAAS

SEQ ID NO: 200 - protein, 25 aa, synthetic construct
  REGION 1..25: note = Description of Artificial Sequence: Synthetic peptide
QVQLVESGGGLVQAGGSLRLSCVAS

SEQ ID NO: 201 - protein, 25 aa, synthetic construct
  REGION 1..25: note = Description of Artificial Sequence: Synthetic peptide
QVQLVESGGGLVQARGSLRLSCTAS

SEQ ID NO: 202 - protein, 25 aa, synthetic construct
  REGION 1..25: note = Description of Artificial Sequence: Synthetic peptide
QVQLVESGGGLVQAGGSLRLSCAAS

SEQ ID NO: 203 - protein, 25 aa, synthetic construct
  REGION 1..25: note = Description of Artificial Sequence: Synthetic peptide
QVQLVESGGGLVQAGGSLRLSCAAS

SEQ ID NO: 204 - protein, 25 aa, synthetic construct
  REGION 1..25: note = Description of Artificial Sequence: Synthetic peptide
QVQLVESGGGLVQAGGSLRLSCTTS

SEQ ID NO: 205 - protein, 25 aa, synthetic construct
  REGION 1..25: note = Description of Artificial Sequence: Synthetic peptide
QVQLVESGGGLVQAGGSLTLSCAAS

SEQ ID NO: 206 - protein, 25 aa, synthetic construct
  REGION 1..25: note = Description of Artificial Sequence: Synthetic peptide
QVQLVESGGGLVQAGGSLRLSCVAS

SEQ ID NO: 207 - protein, 25 aa, synthetic construct
  REGION 1..25: note = Description of Artificial Sequence: Synthetic peptide
QVQLVESGGGLVQAGGSLRLSCAAS

SEQ ID NO: 208 - protein, 25 aa, synthetic construct [sequence omitted]

SEQ ID NO: 209 - protein, 25 aa, synthetic construct [sequence omitted]

SEQ ID NO: 210 - protein, 25 aa, synthetic construct
  REGION 1..25: note = Description of Artificial Sequence: Synthetic peptide
EVQLVESGGGLVQAGGSLRLSCAAS

SEQ ID NO: 211 - protein, 25 aa, synthetic construct [sequence omitted]

SEQ ID NO: 212 - protein, 25 aa, synthetic construct [sequence omitted]

SEQ ID NO: 213 - protein, 25 aa, synthetic construct
  REGION 1..25: note = Description of Artificial Sequence: Synthetic peptide
QVQLVESGGGVVQAGGSLRLSCAAS

SEQ ID NO: 214 - protein, 25 aa, synthetic construct
  REGION 1..25: note = Description of Artificial Sequence: Synthetic peptide
QVQLVESGGGVVQPGGSLRLSCAAS

SEQ ID NO: 215 - protein, 25 aa, synthetic construct
  REGION 1..25: note = Description of Artificial Sequence: Synthetic peptide
QVQLVESGGGVVQPGGSLRLSCAAS

SEQ ID NO: 216 - protein, 25 aa, synthetic construct
  REGION 1..25: note = Description of Artificial Sequence: Synthetic peptide
EVQLVESGGGLVQAGGSLRLSCVAS

SEQ ID NO: 217 - protein, 25 aa, synthetic construct [sequence omitted]

SEQ ID NO: 218 - protein, 25 aa, synthetic construct [sequence omitted]

SEQ ID NO: 219 - protein, 13 aa, synthetic construct
  REGION 1..13: note = Description of Artificial Sequence: Synthetic peptide
WYRQAGNNRALVA

SEQ ID NO: 220 - protein, 14 aa, synthetic construct
  REGION 1..14: note = Description of Artificial Sequence: Synthetic peptide
WYRQAPGKQRELVA

SEQ ID NO: 221 - protein, 14 aa, synthetic construct
  REGION 1..14: note = Description of Artificial Sequence: Synthetic peptide
WYRQAPGKQRTVVA

SEQ ID NO: 222 - protein, 14 aa, synthetic construct
  REGION 1..14: note = Description of Artificial Sequence: Synthetic peptide
WYRQAPGKERELVA

SEQ ID NO: 223 - protein, 14 aa, synthetic construct
  REGION 1..14: note = Description of Artificial Sequence: Synthetic peptide
WYRQAPGKQRELVA

SEQ ID NO: 224 - protein, 14 aa, synthetic construct
  REGION 1..14: note = Description of Artificial Sequence: Synthetic peptide
WYRQDPSKQREWVA

SEQ ID NO: 225 - protein, 14 aa, synthetic construct
  REGION 1..14: note = Description of Artificial Sequence: Synthetic peptide
WYRQAPGKQRELVA

SEQ ID NO: 226 - protein, 14 aa, synthetic construct
  REGION 1..14: note = Description of Artificial Sequence: Synthetic peptide
WYRQASGKERESVA

SEQ ID NO: 227 - protein, 14 aa, synthetic construct
  REGION 1..14: note = Description of Artificial Sequence: Synthetic peptide
WYRQASGKERESVA

SEQ ID NO: 228 - protein, 14 aa, synthetic construct
  REGION 1..14: note = Description of Artificial Sequence: Synthetic peptide
WVRQAPGKGLEWVS

SEQ ID NO: 229 - protein, 14 aa, synthetic construct
  REGION 1..14: note = Description of Artificial Sequence: Synthetic peptide
WYRQAPGKEREMVA

SEQ ID NO: 230 - protein, 14 aa, synthetic construct
  REGION 1..14: note = Description of Artificial Sequence: Synthetic peptide
WFRQAPGKQREWVA

SEQ ID NO: 231 - protein, 14 aa, synthetic construct
  REGION 1..14: note = Description of Artificial Sequence: Synthetic peptide
WYRQAQGKQREPVA

SEQ ID NO: 232 - protein, 14 aa, synthetic construct
  REGION 1..14: note = Description of Artificial Sequence: Synthetic peptide
WFRQAPGKERQFVA

SEQ ID NO: 233 - protein, 14 aa, synthetic construct
  REGION 1..14: note = Description of Artificial Sequence: Synthetic peptide
WHRQAPGKQREPVA

SEQ ID NO: 234 - protein, 14 aa, synthetic construct
  REGION 1..14: note = Description of Artificial Sequence: Synthetic peptide
WYRRAPGQVREMVA

SEQ ID NO: 235 - protein, 14 aa, synthetic construct
  REGION 1..14: note = Description of Artificial Sequence: Synthetic peptide
WYRQAPGTERDLVA

SEQ ID NO: 236 - protein, 14 aa, synthetic construct
  REGION 1..14: note = Description of Artificial Sequence: Synthetic peptide
WYRQAPGAQRELLA

SEQ ID NO: 237 - protein, 14 aa, synthetic construct
  REGION 1..14: note = Description of Artificial Sequence: Synthetic peptide
WYRQAPGKQRELVA

SEQ ID NO: 238 - protein, 14 aa, synthetic construct
  REGION 1..14: note = Description of Artificial Sequence: Synthetic peptide
WYRQAPGKERELVA

SEQ ID NO: 239 - protein, 14 aa, synthetic construct
  REGION 1..14: note = Description of Artificial Sequence: Synthetic peptide
WYRQAPGKQRELVA

SEQ ID NO: 240 - protein, 14 aa, synthetic construct
  REGION 1..14: note = Description of Artificial Sequence: Synthetic peptide
WYRQAPGNQRELVA

SEQ ID NO: 241 - protein, 14 aa, synthetic construct
  REGION 1..14: note = Description of Artificial Sequence: Synthetic peptide
WYRQAPGKQREPVA

SEQ ID NO: 242 - protein, 14 aa, synthetic construct
  REGION 1..14: note = Description of Artificial Sequence: Synthetic peptide
WFRQAPGEERELVA

SEQ ID NO: 243 - protein, 14 aa, synthetic construct
  REGION 1..14: note = Description of Artificial Sequence: Synthetic peptide
WYRQAPGNQREPVA

SEQ ID NO: 244 - protein, 14 aa, synthetic construct
  REGION 1..14: note = Description of Artificial Sequence: Synthetic peptide
WYRQAPGTERDLVA

SEQ ID NO: 245 - protein, 14 aa, synthetic construct
  REGION 1..14: note = Description of Artificial Sequence: Synthetic peptide
WHRQAPGNERELVA

SEQ ID NO: 246 - protein, 14 aa, synthetic construct
  REGION 1..14: note = Description of Artificial Sequence: Synthetic peptide
WYRQAPGKERELVA

SEQ ID NO: 247 - protein, 14 aa, synthetic construct
  REGION 1..14: note = Description of Artificial Sequence: Synthetic peptide
WYRQAPGKQRELVA

SEQ ID NO: 248 - protein, 14 aa, synthetic construct
  REGION 1..14: note = Description of Artificial Sequence: Synthetic peptide
WVRQAPGKGLEWVS

SEQ ID NO: 249 - protein, 14 aa, synthetic construct
  REGION 1..14: note = Description of Artificial Sequence: Synthetic peptide
WYRQAPGKERELVA

SEQ ID NO: 250 - protein, 14 aa, synthetic construct
  REGION 1..14: note = Description of Artificial Sequence: Synthetic peptide
WYRQAPGKERELVA

SEQ ID NO: 251 - protein, 14 aa, synthetic construct
  REGION 1..14: note = Description of Artificial Sequence: Synthetic peptide
WVRQAPGKGLEWVS

SEQ ID NO: 252 - protein, 14 aa, synthetic construct
  REGION 1..14: note = Description of Artificial Sequence: Synthetic peptide
WYRQAPGTERDLVA

SEQ ID NO: 253 - protein, 14 aa, synthetic construct
  REGION 1..14: note = Description of Artificial Sequence: Synthetic peptide
WYRQAPGKERELVA

SEQ ID NO: 254 - protein, 14 aa, synthetic construct
  REGION 1..14: note = Description of Artificial Sequence: Synthetic peptide
WVRQAPGKGLEWVS

SEQ ID NO: 255 - protein, 14 aa, synthetic construct
  REGION 1..14: note = Description of Artificial Sequence: Synthetic peptide
WHRQAPGNERELVA

SEQ ID NO: 256 - protein, 14 aa, synthetic construct
  REGION 1..14: note = Description of Artificial Sequence: Synthetic peptide
WHRQAPGKERELVA

SEQ ID NO: 257 - protein, 14 aa, synthetic construct
  REGION 1..14: note = Description of Artificial Sequence: Synthetic peptide
WVRQAPGKGLEWVS

SEQ ID NO: 258 - protein, 29 aa, synthetic construct
  REGION 1..29: note = Description of Artificial Sequence: Synthetic peptide
ISWDIAENTVYLQMNSLNSEDTTVYYCNS

SEQ ID NO: 259 - protein, 29 aa, synthetic construct
  REGION 1..29: note = Description of Artificial Sequence: Synthetic peptide
ISGDNVRNMVYLQMNSLKPEDTAIYYCSA

SEQ ID NO: 260 - protein, 29 aa, synthetic construct
  REGION 1..29: note = Description of Artificial Sequence: Synthetic peptide
ISGENGKNTVYLQMNSLKLEDTAVYYCLG

SEQ ID NO: 261 - protein, 29 aa, synthetic construct
  REGION 1..29: note = Description of Artificial Sequence: Synthetic peptide
ISRDNANNAIYLEMNSLKPEDTAVYVCNA

SEQ ID NO: 262 - protein, 29 aa, synthetic construct
  REGION 1..29: note = Description of Artificial Sequence: Synthetic peptide
ISRDNAENTVSLQMNTLKPEDTAVYFCNA

SEQ ID NO: 263 - protein, 29 aa, synthetic construct
  REGION 1..29: note = Description of Artificial Sequence: Synthetic peptide
ISKDSTRNTVYLQMNMLRPEDTAVYYCNA

SEQ ID NO: 264 - protein, 29 aa, synthetic construct
  REGION 1..29: note = Description of Artificial Sequence: Synthetic peptide
ISRDNAKNTVYLQMNSLKPEDTAVYYCNA

SEQ ID NO: 265 - protein, 29 aa, synthetic construct
  REGION 1..29: note = Description of Artificial Sequence: Synthetic peptide
ISRDIDKKTVYLQMDNLKPEDTGVYYCNS

SEQ ID NO: 266 - protein, 29 aa, synthetic construct
  REGION 1..29: note = Description of Artificial Sequence: Synthetic peptide
ISRDIYKKTVYLQMDNLKPEDTGVYYCNS

SEQ ID NO: 267 - protein, 29 aa, synthetic construct
  REGION 1..29: note = Description of Artificial Sequence: Synthetic peptide
ISRDNAKTTLYLQMNSLRPEDTAVYYCTI

SEQ ID NO: 268 - protein, 29 aa, synthetic construct
  REGION 1..29: note = Description of Artificial Sequence: Synthetic peptide
ISKENAKNTVYLQMNSLKPEDTAVYYCHI

SEQ ID NO: 269 - protein, 29 aa, synthetic construct
  REGION 1..29: note = Description of Artificial Sequence: Synthetic peptide
ISRDNAKSTVYLQMNSLKPEDTAIYYCKA

SEQ ID NO: 270 - protein, 29 aa, synthetic construct
  REGION 1..29: note = Description of Artificial Sequence: Synthetic peptide
ISRDNAKNTVYLQMNSLKPEDTAIYTCHV

SEQ ID NO: 271 - protein, 29 aa, synthetic construct
  REGION 1..29: note = Description of Artificial Sequence: Synthetic peptide
ISRDNAANTFYLQMNNLRPDDTAVYYCNV

SEQ ID NO: 272 - protein, 29 aa, synthetic construct
  REGION 1..29: note = Description of Artificial Sequence: Synthetic peptide
VSRDIVKNTMYLQMNSLKPEDTAVYYCSY

SEQ ID NO: 273 - protein, 29 aa, synthetic construct
  REGION 1..29: note = Description of Artificial Sequence: Synthetic peptide
ISRDNTQNLVYLQMNNLQPHDTAIYYCGA

SEQ ID NO: 274 - protein, 29 aa, synthetic construct
  REGION 1..29: note = Description of Artificial Sequence: Synthetic peptide
ISRDNAKNTLYLQMNNLKPEDTAVYYCNA

SEQ ID NO: 275 - protein, 29 aa, synthetic construct
  REGION 1..29: note = Description of Artificial Sequence: Synthetic peptide
ISRDNAKNTVYLQMNRLTPEDTDVYYCRF

SEQ ID NO: 276 - protein, 29 aa, synthetic construct
  REGION 1..29: note = Description of Artificial Sequence: Synthetic peptide
ISRDNAKNTVYLQMESLVAEDTAVYYCNA

SEQ ID NO: 277 - protein, 29 aa, synthetic construct
  REGION 1..29: note = Description of Artificial Sequence: Synthetic peptide
VSRDSAKNIVYLQMNSLTPEDTAVYYCNT

SEQ ID NO: 278 - protein, 29 aa, synthetic construct
  REGION 1..29: note = Description of Artificial Sequence: Synthetic peptide
ISRDNAKNTVYLQMNSLKPEDTAVYYCNA

SEQ ID NO: 279 - protein, 29 aa, synthetic construct
  REGION 1..29: note = Description of Artificial Sequence: Synthetic peptide
ISRDNAKNTVYLQMNSLKPEDTAVYYCNT

SEQ ID NO: 280 - protein, 29 aa, synthetic construct
  REGION 1..29: note = Description of Artificial Sequence: Synthetic peptide
ISRDNAKNTVYLQMNSLKPEDTAIYTCHV

SEQ ID NO: 281 - protein, 29 aa, synthetic construct
  REGION 1..29: note = Description of Artificial Sequence: Synthetic peptide
VSRDSAKNIVYLQMNRLKPEDTAVYYCNT

SEQ ID NO: 282 - protein, 29 aa, synthetic construct
  REGION 1..29: note = Description of Artificial Sequence: Synthetic peptide
ISRDNARNTVYLQMDSLKPEDTAIYTCHV

SEQ ID NO: 283 - protein, 29 aa, synthetic construct
  REGION 1..29: note = Description of Artificial Sequence: Synthetic peptide
ISRDNAKNTLYLQMNNLKPEDTAVYYCNA

SEQ ID NO: 284 - protein, 29 aa, synthetic construct
  REGION 1..29: note = Description of Artificial Sequence: Synthetic peptide
ISRDNAKNTAYLQMNSLKPEDTAVYYCSV

SEQ ID NO: 285 - protein, 29 aa, synthetic construct
  REGION 1..29: note = Description of Artificial Sequence: Synthetic peptide
VSRDSAKNIVYLQMNSLKPEDTAVYYCNT

SEQ ID NO: 286 - protein, 29 aa, synthetic construct
  REGION 1..29: note = Description of Artificial Sequence: Synthetic peptide
ISRDNSKNTLYLQMNSLRAEDTAVYYCAV

SEQ ID NO: 287 - protein, 29 aa, synthetic construct
  REGION 1..29: note = Description of Artificial Sequence: Synthetic peptide
ISRDNSKNTLYLQMNSLRAEDTAVYYCAV

SEQ ID NO: 288 - protein, 29 aa, synthetic construct
  REGION 1..29: note = Description of Artificial Sequence: Synthetic peptide
ISRDNSKNTLYLQMNSLRAEDTAVYYCNT

SEQ ID NO: 289 - protein, 29 aa, synthetic construct
  REGION 1..29: note = Description of Artificial Sequence: Synthetic peptide
ISRDNSKNTLYLQMNSLRAEDTAVYYCNT

SEQ ID NO: 290 - protein, 29 aa, synthetic construct
  REGION 1..29: note = Description of Artificial Sequence: Synthetic peptide
ISRDNSKNTLYLQMNSLRAEDTAVYYCNT

SEQ ID NO: 291 - protein, 29 aa, synthetic construct
  REGION 1..29: note = Description of Artificial Sequence: Synthetic peptide
ISRDNSKNTLYLQMNSLRAEDTAVYYCNA

SEQ ID NO: 292 - protein, 29 aa, synthetic construct
  REGION 1..29: note = Description of Artificial Sequence: Synthetic peptide
ISRDNSKNTLYLQMNSLRAEDTAVYYCNA

SEQ ID NO: 293 - protein, 29 aa, synthetic construct
  REGION 1..29: note = Description of Artificial Sequence: Synthetic peptide
ISRDNSKNTLYLQMNSLRAEDTAVYYCNA

SEQ ID NO: 294 - protein, 29 aa, synthetic construct
  REGION 1..29: note = Description of Artificial Sequence: Synthetic peptide
ISRDNSKNTLYLQMNSLRAEDTAVYYCSV

SEQ ID NO: 295 - protein, 29 aa, synthetic construct
  REGION 1..29: note = Description of Artificial Sequence: Synthetic peptide
ISRDNSKNTLYLQMNSLRAEDTAVYYCSV

SEQ ID NO: 296 - protein, 29 aa, synthetic construct
  REGION 1..29: note = Description of Artificial Sequence: Synthetic peptide
ISRDNSKNTLYLQMNSLRAEDTAVYYCSV

SEQ ID NO: 297 - protein, 11 aa, synthetic construct
  REGION 1..11: note = Description of Artificial Sequence: Synthetic peptide
WGQGTQVTVSS

SEQ ID NO: 298 - protein, 11 aa, synthetic construct
  REGION 1..11: note = Description of Artificial Sequence: Synthetic peptide
WGKGTLVTVSS

SEQ ID NO: 299 - protein, 11 aa, synthetic construct
  REGION 1..11: note = Description of Artificial Sequence: Synthetic peptide
WGQGTQVTVSS

SEQ ID NO: 300 - protein, 11 aa, synthetic construct
  REGION 1..11: note = Description of Artificial Sequence: Synthetic peptide
FGQGTQVTVSS

SEQ ID NO: 301 - protein, 11 aa, synthetic construct
  REGION 1..11: note = Description of Artificial Sequence: Synthetic peptide
WGQGTQVTVSS

SEQ ID NO: 302 - protein, 11 aa, synthetic construct
  REGION 1..11: note = Description of Artificial Sequence: Synthetic peptide
WGQGTQVTVSS

SEQ ID NO: 303 - protein, 11 aa, synthetic construct
  REGION 1..11: note = Description of Artificial Sequence: Synthetic peptide
WGQGTQVTVSS

SEQ ID NO: 304 - protein, 11 aa, synthetic construct
  REGION 1..11: note = Description of Artificial Sequence: Synthetic peptide
WGQGTQVTVSS

SEQ ID NO: 305 - protein, 11 aa, synthetic construct
  REGION 1..11: note = Description of Artificial Sequence: Synthetic peptide
WGQGTQVTVSS

SEQ ID NO: 306 - protein, 9 aa, synthetic construct
  REGION 1..9: note = Description of Artificial Sequence: Synthetic peptide
QGTLVTVSS

SEQ ID NO: 307 - protein, 11 aa, synthetic construct
  REGION 1..11: note = Description of Artificial Sequence: Synthetic peptide
WGQGTQVTVSS

SEQ ID NO: 308 - protein, 11 aa, synthetic construct
  REGION 1..11: note = Description of Artificial Sequence: Synthetic peptide
WGQGTQVTVSS

SEQ ID NO: 309 - protein, 11 aa, synthetic construct
  REGION 1..11: note = Description of Artificial Sequence: Synthetic peptide
WGQGTQVTVSS

SEQ ID NO: 310 - protein, 11 aa, synthetic construct
  REGION 1..11: note = Description of Artificial Sequence: Synthetic peptide
WGQGTQVTVSS

SEQ ID NO: 311 - protein, 11 aa, synthetic construct
  REGION 1..11: note = Description of Artificial Sequence: Synthetic peptide
WGQGTQVTVSS

SEQ ID NO: 312 - protein, 11 aa, synthetic construct
  REGION 1..11: note = Description of Artificial Sequence: Synthetic peptide
WGQGTQVTVSS

SEQ ID NO: 313 - protein, 11 aa, synthetic construct
  REGION 1..11: note = Description of Artificial Sequence: Synthetic peptide
WGQGTQVTVSS

SEQ ID NO: 314 - protein, 11 aa, synthetic construct
  REGION 1..11: note = Description of Artificial Sequence: Synthetic peptide
WGQGTQVTVSS

SEQ ID NO: 315 - protein, 11 aa, synthetic construct
  REGION 1..11: note = Description of Artificial Sequence: Synthetic peptide
WGQGTQVTVSS

SEQ ID NO: 316 - protein, 11 aa, synthetic construct
  REGION 1..11: note = Description of Artificial Sequence: Synthetic peptide
WGQGTQVTVSS

SEQ ID NO: 317 - protein, 11 aa, synthetic construct
  REGION 1..11: note = Description of Artificial Sequence: Synthetic peptide
WGQGTQVTVSS

SEQ ID NO: 318 - protein, 11 aa, synthetic construct
  REGION 1..11: note = Description of Artificial Sequence: Synthetic peptide
WGQGTQVTVSS

SEQ ID NO: 319 - protein, 11 aa, synthetic construct
  REGION 1..11: note = Description of Artificial Sequence: Synthetic peptide
WGQGTQVTVSS

SEQ ID NO: 320 - protein, 11 aa, synthetic construct
  REGION 1..11: note = Description of Artificial Sequence: Synthetic peptide
WGQGTQVTVSS

SEQ ID NO: 321 - protein, 11 aa, synthetic construct
  REGION 1..11: note = Description of Artificial Sequence: Synthetic peptide
WGQGTQVTVSS

SEQ ID NO: 322 - protein, 11 aa, synthetic construct
  REGION 1..11: note = Description of Artificial Sequence: Synthetic peptide
WGQGTQVTVSS

SEQ ID NO: 323 - protein, 11 aa, synthetic construct
  REGION 1..11: note = Description of Artificial Sequence: Synthetic peptide
WGQGTQVTVSS

SEQ ID NO: 324 - protein, 11 aa, synthetic construct
  REGION 1..11: note = Description of Artificial Sequence: Synthetic peptide
WGQGTQVTVSS

SEQ ID NO: 325 - protein, 11 aa, synthetic construct
  REGION 1..11: note = Description of Artificial Sequence: Synthetic peptide
WGQGTLVTVSS

SEQ ID NO: 326 - protein, 11 aa, synthetic construct
  REGION 1..11: note = Description of Artificial Sequence: Synthetic peptide
WGQGTLVTVSS

SEQ ID NO: 327 - protein, 11 aa, synthetic construct
  REGION 1..11: note = Description of Artificial Sequence: Synthetic peptide
WGQGTLVTVSS

SEQ ID NO: 328 - protein, 11 aa, synthetic construct
  REGION 1..11: note = Description of Artificial Sequence: Synthetic peptide
WGQGTLVTVSS

SEQ ID NO: 329 - protein, 11 aa, synthetic construct
  REGION 1..11: note = Description of Artificial Sequence: Synthetic peptide
WGQGTLVTVSS

SEQ ID NO: 330 - protein, 13 aa, synthetic construct
  REGION 1..13: note = Description of Artificial Sequence: Synthetic peptide
WGQGTLVTVSSGG

SEQ ID NO: 331 - protein, 13 aa, synthetic construct
  REGION 1..13: note = Description of Artificial Sequence: Synthetic peptide
WGQGTLVTVSSGG

SEQ ID NO: 332 - protein, 13 aa, synthetic construct
  REGION 1..13: note = Description of Artificial Sequence: Synthetic peptide
WGQGTLVTVSSGG

SEQ ID NO: 333 - protein, 10 aa, synthetic construct
  REGION 1..10: note = Description of Artificial Sequence: Synthetic peptide
WGQGTLVTVS

SEQ ID NO: 334 - protein, 11 aa, synthetic construct
  REGION 1..11: note = Description of Artificial Sequence: Synthetic peptide
WGQGTLVTVSS

SEQ ID NO: 335 - protein, 11 aa, synthetic construct
  REGION 1..11: note = Description of Artificial Sequence: Synthetic peptide
WGQGTLVTVSS

SEQ ID NO: 336 - protein, 6 aa, synthetic construct
  REGION 1..6: note = Description of Artificial Sequence: Synthetic 6xHis tag
HHHHHH